(19) United States
(12) Patent Application Publication
(10) Pub. No.: US 2022/0110975 A1
(43) Pub. Date: Apr. 14, 2022

(54) METHOD FOR TREATING DISEASE USING FOXP3+CD4+ T CELLS

(71) Applicant: Kyverna Therapeutics, Inc., Berkeley, CA (US)

(72) Inventors: Sarah Levinson, Berkeley, CA (US); John Lee, Berkeley, CA (US); Jordan Tsai, Berkeley, CA (US); Jeanne Flandez, Berkeley, CA (US); Ashley Mahne, Berkeley, CA (US); Faye Wu, Berkeley, CA (US); Sasha Farina, Berkeley, CA (US); Harsh Srivastava, Berkeley, CA (US); Joseph Park, Berkeley, CA (US); Jeffrey Greve, Berkeley, CA (US); Fred Cohen, Berkeley, CA (US)

(21) Appl. No.: 17/499,157

(22) Filed: Oct. 12, 2021

Related U.S. Application Data
(60) Provisional application No. 63/090,539, filed on Oct. 12, 2020.

(51) Int. Cl. A61K 35/17; C07K 14/47; C12N 15/113; C07K 14/71; C12N 5/0783; C12N 15/86

(52) U.S. Cl. CPC A61K 35/17; C07K 14/4702; C12N 15/113; C07K 14/71; C12N 2740/15043; C12N 15/86; C12N 2310/141; C12N 2510/00; C12N 5/0636

(57) ABSTRACT

This document relates to methods and materials for treating a mammal having an autoimmune disease. For example, materials and methods for producing a T cell comprising a FOXP3 polypeptide and a microRNA are provided herein. Also provided are methods of treating an autoimmune disease that include administering these T cells.

### CROSS-REFERENCE TO RELATED APPLICATIONS

This application claims priority to U.S. Provisional Patent Application 63/090,539, filed on Oct. 12, 2020. The contents of that application are incorporated herein by reference in its entirety.

### SEQUENCE LISTING

This application contains a Sequence Listing that has been submitted electronically as an ASCII text file named 47902_0015001_SEQ.txt. The ASCII text file, created on Oct. 12, 2021, is 26.2 kilobytes in size. The material in the ASCII text file is hereby incorporated by reference in its entirety.

### BACKGROUND

This document relates to methods and materials for treating a mammal having an autoimmune disease. For example, this document provides materials and methods for producing a T cell comprising a forkhead box P3 (FOXP3) polypeptide and a microRNA (miRNA). This document also provides methods and materials for treating a mammal having an autoimmune disease, where the methods include administering to a mammal having an autoimmune disease an effective amount of the T cell.

Autoimmunity is a common disease in the United States, with more than 20 million people suffering from one of 81 known autoimmune diseases. Regulatory T cells (Tregs) are a subpopulation of T cells that modulate the immune system and maintain tolerance to self-antigens. Tregs play a role in preventing or treating autoimmune disease (Sakaguchi et al., Int'l Immun., 21(10):1105-1111 (2009)). FOXP3, a transcription factor expressed in Tregs, has been implicated in maintaining Treg immunosuppressive functions (Hort et al., Science, 299:1057-1061 (2003)).

### SUMMARY

Provided herein are methods and materials that can be used to treat mammals identified as having an autoimmune disease. For example, provided herein are materials and methods for generating a T cell containing a FOXP3 polypeptide and a microRNA (miRNA) (e.g., one or more of any of the exemplary T cells described herein). Also provided herein are methods and materials for treating a mammal having an autoimmune disease that include administering to the mammal an effective amount of a T cell (e.g., any of the T cells described herein). The methods and materials provided herein can provide a way to enhance and/or stabilize the immunosuppressive effects of a T cell in order to treat the autoimmune disease.

Provided herein are methods for increasing T cell function, that include: introducing into a T cell: (i) a first nucleic acid sequence encoding a FOXP3 polypeptide; and (ii) a second nucleic acid sequence encoding a microRNA. In some embodiments of any of the methods described herein, the FOXP3 polypeptide comprises a sequence that is at least 80% identical to SEQ ID NO: 1. In some embodiments of any of the methods described herein, the FOXP3 polypeptide comprises a sequence that is at least 90% identical to SEQ ID NO: 1. In some embodiments of any of the methods described herein, the FOXP3 polypeptide comprises a sequence that is at least 95% identical to SEQ ID NO: 1. In some embodiments of any of the methods described herein, the FOXP3 polypeptide comprises SEQ ID NO: 1.

In some embodiments of any of the methods described herein, the microRNA is selected form the group of: miR-142, miR-155, miR-15b, miR-16, miR-146a, miR-21a, miR-99a, miR-150, miR-10a, miR-95, miR-126, miR-29a, miR-24, miR-181c, and miR-101. In some embodiments of any of the methods described herein, the microRNA is miR-142. In some embodiments of any of the methods described herein, the microRNA is miR-155. In some embodiments of any of the methods described herein, the microRNA is miR-15b. In some embodiments of any of the methods described herein, the microRNA is miR-16. In some embodiments of any of the methods described herein, the microRNA is miR-146a. In some embodiments of any of the methods described herein, the microRNA is miR-21a. In some embodiments of any of the methods described herein, the microRNA is miR-99a. In some embodiments of any of the methods described herein, the microRNA is miR-150. In some embodiments of any of the methods described herein, the microRNA is miR-10a. In some embodiments of any of the methods described herein, the microRNA is miR-95. In some embodiments of any of the methods described herein, the microRNA is miR-126. In some embodiments of any of the methods described herein, the microRNA is miR-29a. In some embodiments of any of the methods described herein, the microRNA is miR-24. In some embodiments of any of the methods described herein, the microRNA is miR-181c. In some embodiments of any of the methods described herein, the microRNA is miR-101.

In some embodiments of any of the methods described herein, the T cell, after the introducing, has one or more of the following activities: increased levels of FoxP3 mRNA and/or FoxP3 protein, increased levels of CD25 mRNA and/or protein, and increased levels of CTLA4 mRNA and/or CTLA4 protein, as compared to a T cell including the first nucleic acid, but not including the second nucleic acid.

In some embodiments of any of the methods described herein, the first nucleic acid sequence further comprises a nucleic acid sequence encoding a truncated nerve growth factor receptor (tNGFR) polypeptide. In some embodiments of any of the methods described herein, the second nucleic acid sequence comprises a nucleic acid sequence encoding a reporter polypeptide. In some embodiments of any of the methods described herein, the reporter is an eGFP polypeptide.

In some embodiments of any of the methods described herein, the introducing step further comprises introducing a nucleic acid construct, where the nucleic acid construct comprises the first nucleic acid sequence and the second nucleic acid sequence. In some embodiments of any of the methods described herein, the nucleic acid construct further comprises a promoter operably linked to the first nucleic acid sequence.

In some embodiments of any of the methods described herein, the first nucleic acid sequence is 5′ positioned relative to the second nucleic acid in the nucleic acid construct. In some embodiments of any of the methods described herein, the nucleic acid construct further comprises an additional nucleic acid sequence between the first nucleic acid sequence and the second nucleic acid sequence, where the additional nucleic acid sequence operably links the second nucleic acid sequence to the first nucleic acid sequence.

In some embodiments of any of the methods described herein, the second nucleic acid sequence is 5′ positioned relative to the first nucleic acid in the nucleic acid construct. In some embodiments of any of the methods described herein, the nucleic acid construct further comprises an additional nucleic acid sequence between the second nucleic acid sequence and the first nucleic acid sequence, where the additional nucleic acid sequence operably links the first nucleic acid sequence to the second nucleic acid sequence.

In some embodiments of any of the methods described herein, the additional nucleic acid sequence encodes an internal ribosome entry site (IRES) sequence or a self-cleaving amino acid. In some embodiments of any of the methods described herein, the additional nucleic acid sequence comprises a promoter or enhancer.

In some embodiments of any of the methods described herein, the nucleic acid construct is a viral vector selected from the group of a lentiviral vector, a retroviral vector, an adenoviral vector, or an adeno-associated viral (AAV) vector. In some embodiments of any of the methods described herein, the viral vector is a lentiviral vector. In some embodiments of any of the methods described herein, the introducing step comprises viral transduction.

In some embodiments of any of the methods described herein, the T cell is a CD4⁺T cell or a CD4⁺/CD45RA⁺ T cell.

Some embodiments of any of the methods described herein further include: obtaining a T cell from a patient or obtaining T cells allogenic to the patient. Some embodiments of any of the methods described herein further include: treating the obtained T cells to isolate a population of cells enriched for CD4⁺ T cells or CD4⁺/CD45RA⁺ T cells.

Also provided herein is a T cell produced by any of the methods described herein. Also provided herein are compositions that include a T cell produced by any of the methods described herein.

Also provided herein are T cells that include: a first nucleic acid sequence encoding a FOXP3 polypeptide; and a second nucleic acid sequence encoding a microRNA. In some embodiments of any of the T cells described herein, the FOXP3 polypeptide comprises a sequence that is at least 80% identical to SEQ ID NO: 1. In some embodiments of any of the T cells described herein, the FOXP3 polypeptide comprises a sequence that is at least 90% identical to SEQ ID NO: 1. In some embodiments of any of the T cells described herein, the FOXP3 polypeptide comprises a sequence that is at least 95% identical to SEQ ID NO: 1. In some embodiments of any of the T cells described herein, the FOXP3 polypeptide comprises SEQ ID NO: 1.

In some embodiments of any of the T cells described herein, the microRNA is selected form the group of: miR-142, miR-155, miR-15b, miR-16, miR-146a, miR-21a, miR-99a, miR-150, miR-10a, miR-95, miR-126, miR-29a, miR-24, miR-181c, and miR-101. In some embodiments of any of the T cells described herein, the microRNA is miR-142. In some embodiments of any of the T cells described herein, the microRNA is miR-155. In some embodiments of any of the T cells described herein, the microRNA is miR-15b. In some embodiments of any of the T cells described herein, the microRNA is miR-16. In some embodiments of any of the T cells described herein, the microRNA is miR-146a. In some embodiments of any of the T cells described herein, the microRNA is miR-21a. In some embodiments of any of the T cells described herein, the microRNA is miR-99a. In some embodiments of any of the T cells described herein, the microRNA is miR-150. In some embodiments of any of the T cells described herein, the microRNA is miR-10a. In some embodiments of any of the T cells described herein, the microRNA is miR-95. In some embodiments of any of the T cells described herein, the microRNA is miR-126. In some embodiments of any of the T cells described herein, the microRNA is miR-29a. In some embodiments of any of the T cells described herein, the microRNA is miR-24. In some embodiments of any of the T cells described herein, the microRNA is miR-181c. In some embodiments of any of the T cells described herein, the microRNA is miR-101.

In some embodiments of any of the T cells described herein, the T cell, after the introducing, has one or more of the following activities: increased levels of FoxP3 mRNA and/or FoxP3 protein, increased levels of CD25 mRNA and/or CD25 protein, and increased levels of CTLA4 mRNA and/or CTLA4 protein, as compared to a T cell including the first nucleic acid, but not including the second nucleic acid.

In some embodiments of any of the T cells described herein, the first nucleic acid sequence further comprises a nucleic acid sequence encoding a truncated nerve growth factor receptor (tNGFR) polypeptide. In some embodiments of any of the T cells described herein, the second nucleic acid sequence comprises a nucleic acid sequence encoding a reporter polypeptide. In some embodiments of any of the T cells described herein, the reporter is an eGFP polypeptide.

In some embodiments of any of the T cells described herein, the introducing step further comprises introducing a nucleic acid construct, wherein the nucleic acid construct comprises the first nucleic acid sequence and the second nucleic acid sequence. In some embodiments of any of the T cells described herein, the first nucleic acid sequence is operably linked to a promoter. In some embodiments of any of the T cells described herein, the second nucleic acid sequence is operably linked to a promoter.

Also provided herein are compositions that include any of the T cells described herein.

Also provided herein are methods of producing a T cell population expressing an exogenous FOXP3 polypeptide and a microRNA that include culturing any of the T cells described herein in growth media under conditions sufficient to expand the population of T cells.

Also provided herein is a population of T cells prepared using any of the methods described herein. Also provided herein are compositions that include a population of T cells prepared using any of the methods described herein.

Also provided herein are vectors that include a first nucleic acid sequence encoding a FOXP3 polypeptide and a second nucleic acid sequence encoding a micro-RNA. In some embodiments of any of the vectors described herein, the FOXP3 polypeptide comprises a sequence that is at least 80% identical to SEQ ID NO: 1. In some embodiments of any of the vectors described herein, the FOXP3 polypeptide comprises a sequence that is at least 90% identical to SEQ ID NO: 1. In some embodiments of any of the vectors described herein, the FOXP3 polypeptide comprises a sequence that is at least 95% identical to SEQ ID NO: 1. In some embodiments of any of the vectors described herein, the FOXP3 polypeptide comprises SEQ ID NO: 1.

In some embodiments of any of the vectors described herein, the microRNA is selected form the group of: miR-142, miR-155, miR-15b, miR-16, miR-146a, miR-21a, miR-99a, miR-150, miR-10a, miR-95, miR-126, miR-29a, miR-24, miR-181c, and miR-101. In some embodiments of any of the vectors described herein, the microRNA is miR-142. In some embodiments of any of the vectors described herein, the microRNA is miR-155. In some embodiments of any of the vectors described herein, the microRNA is miR-15b. In some embodiments of any of the vectors described herein, the microRNA is miR-16. In some embodiments of any of the vectors described herein, the microRNA is miR-146a. In some embodiments of any of the vectors described herein, the microRNA is miR-21a. In some embodiments of any of the vectors described herein, the microRNA is miR-99a. In some embodiments of any of the vectors described herein, the microRNA is miR-150. In some embodiments of any of the vectors described herein, the microRNA is miR-10a. In some embodiments of any of the vectors described herein, the microRNA is miR-95. In some embodiments of any of the vectors described herein, the microRNA is miR-126. In some embodiments of any of the vectors described herein, the microRNA is miR-29a. In some embodiments of any of the vectors described herein, the microRNA is miR-24. In some embodiments of any of the vectors described herein, the microRNA is miR-181c. In some embodiments of any of the vectors described herein, the microRNA is miR-101.

In some embodiments of any of the vectors described herein, the first nucleic acid sequence further comprises a nucleic acid sequence encoding a truncated nerve growth factor receptor (tNGFR) polypeptide. In some embodiments of any of the vectors described herein, the second nucleic acid sequence comprises a nucleic acid sequence encoding a reporter polypeptide. In some embodiments of any of the vectors described herein, the reporter is an eGFP polypeptide.

In some embodiments of any of the vectors described herein, the vector further comprises a promoter operably linked to the first nucleic acid sequence. In some embodiments of any of the vectors described herein, the first nucleic acid sequence is 5′ positioned relative to the second nucleic acid in the vector. In some embodiments of any of the vectors described herein, the vector further comprises an additional nucleic acid sequence between the first nucleic acid sequence and the second nucleic acid sequence, where the additional nucleic acid sequence operably links the second nucleic acid sequence to the first nucleic acid sequence.

In some embodiments of any of the vectors described herein, the second nucleic acid sequence is 5′ positioned relative to the first nucleic acid in the vector. In some embodiments of any of the vectors described herein, the vector further comprises an additional nucleic acid sequence between the second nucleic acid sequence and the first nucleic acid sequence, where the additional nucleic acid sequence operably links the first nucleic acid sequence to the second nucleic acid sequence.

In some embodiments of any of the vectors described herein, the additional nucleic acid sequence encodes an internal ribosome entry site (IRES) sequence or a self-cleaving amino acid. In some embodiments of any of the vectors described herein, the additional nucleic acid sequence comprises a promoter or enhancer.

In some embodiments of any of the vectors described herein, the vector comprises a viral vector selected from the group of a lentiviral vector, a retroviral vector, an adenoviral vector, or an adeno-associated viral (AAV) vector. In some embodiments of any of the vectors described herein, the viral vector is a lentiviral vector.

Also provided herein are compositions that include any of the vectors described herein.

Also provided herein are kits that include any of the compositions described herein.

Also provided herein are methods of treating an autoimmune disease or disorder in a patient comprising administering any of the T cells described herein or any of the compositions described herein. In some embodiments of any of the methods described herein, the subject is previously diagnosed or identified as having an autoimmune disease or disorder. In some embodiments of any of the methods described herein, the autoimmune disease or disorder is lupus, rheumatoid arthritis, multiple sclerosis, insulin dependent diabetes mellitis, myasthenia gravis, Graves disease, autoimmune hemolytic anemia, autoimmune thrombocytopenia purpura, Goodpasture's syndrome, pemphigus vulgaris, acute rheumatic fever, post-streptococcal glomerulonephritis, Crohn's disease, Celiac disease, or polyarteritis nodosa. In some embodiments of any of the methods described herein, administering the T cell comprises intravenous injection or intravenous infusion. In some embodiments of any of the methods described herein, the administering results in amelioration of one or more symptoms of the autoimmune disease or disorder.

Also provided herein are compositions that include any of the vectors described herein. Also provided herein are kits that include any of the compositions described herein.

Also provided herein are methods of treating an autoimmune disease or disorder in a patient including administering any of the T cells described herein, or any of the compositions described herein. In some embodiments, the subject can be previously diagnosed or identified as having an autoimmune disease or disorder. In some embodiments, the autoimmune disease or disorder can be lupus, rheumatoid arthritis, multiple sclerosis, insulin-dependent diabetes mellitis, myasthenia gravis, Graves' disease, autoimmune hemolytic anemia, autoimmune thrombocytopenia purpura, Goodpasture's syndrome, pemphigus vulgaris, acute rheumatic fever, post-streptococcal glomerulonephritis, Crohn's disease, Celiac disease, or polyarteritis nodosa. In some embodiments, the administering of the T cell (e.g., any of the autologous or allogenic T cell populations described herein) or any of the compositions described herein can include intravenous injection or intravenous infusion. In some embodiments, the administering can result in amelioration of one or more symptoms of the autoimmune disease or disorder.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWING

FIG. 1 is a diagram showing an exemplary targetable cell with enforced expression of a FOXP3 polypeptide. Enforced expression of a FOXP3 polypeptide results in a core Treg suppressive program (e.g., IL-2 consumption and increase in CD25 expression, an increase in adenosine, an increase in CD39 expression, and expression of CTLA-4).

FIGS. 2A-2B show a schematic of exemplary lentivirus vector constructs. FIG. 2A shows human FOXP3 and truncated NGFR (tNGFR) or tNGFR under the control of an SFFV promoter. FIG. 2B shows GFP and pri-miRNA under the control of an SFFV promoter. WPRE-Woodchuck Hepatitis Virus (WHP) Posttranscriptional Regulatory Element.

FIG. 3 shows flow cytometry plots measuring GFP and NGFR. Quadrant 1 (Q1) and quadrant 2 (Q2) were sorted. Q1 represents cells transduced with eGFP-miR but not FOXP3-tNGFR. Q2 represents cells doubly transduced with FOXP3-tNGFR and eGFP-miR.

FIG. 4 shows flow cytometry plots for the post-sort analysis.

FIGS. 5A-5B show histograms of transcripts per million (TPM) for different genes in CD4Tn_UT (CD4 no transduction control), nTreg, and CD4Tn_FOXP3 (CD4 transduced with FOXP3). FIG. 5A shows TPMs for high abundance genes including FOXP3, FOXP3_CodOpt (Codon optimized), IL2RA, CTLA4 and IFNG. FIG. 5B shows TPMs for low abundance genes including IL7RA, HELIOS, and ENTPD1 (CD39).

FIG. 6 shows a histogram of mean transcript counts for target genes of the respective miRNA. miRNA (target gene) include: miR101 (CDK8), miR101 (CD95), miR15b (mTOR), miR16 (mTOR), miR150 (mTOR), miR146a (AP1), and miR10a (BCL-6).

FIGS. 7A-7C are histograms showing mean transcript counts for Treg phenotypic markers. FIG. 7A shows a histogram of mean transcript counts for FOXP3 in samples treated with FOXP3+miR16, miR101, miR150, miR24, or miR181c or FOXP3 alone. FIG. 7B shows a histogram of mean transcript counts for CD25 in samples treated with FOXP3+miR16, miR101, miR150, miR24, or miR181c or FOXP3 alone. FIG. 7C shows a histogram of mean transcript counts for CTLA4 in samples treated with FOXP3+miR16, miR101, miR150, miR24, or miR181c or FOXP3 alone.

FIG. 8 shows a panel of activated CD4 T cells transduced with SFFV-FOXP3-TNGFR and either SSFV-miRNA-GFP or one of SSFV-miR10a, miR95, miR29a, miR150, and miR101.

FIG. 9 shows a fluorescent-activated cell sorting (FACS) analysis of activated CD4 T cells transduced with either FOXP3 or FOXP3 and miR-155 and in the presence or absence of interleukin (IL)-2.

FIGS. 10A-B are graphs showing the percent phosphor-STAT5 measured on activated T cells under increasing concentrations of IL-2 transduced with different nucleic acids (FIG. 10A) and the fold expansion of activated T cells transduced with different nucleic acids (FIG. 10B).

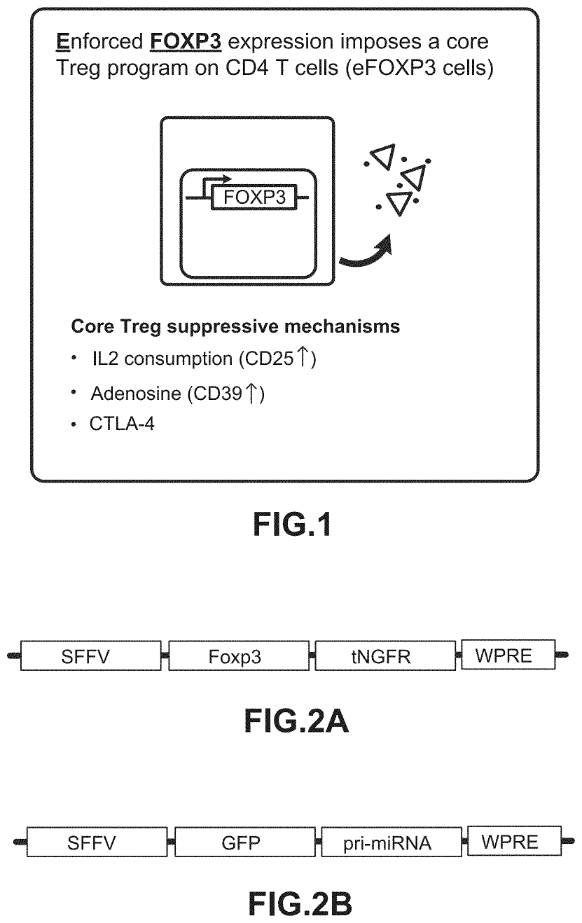

### DETAILED DESCRIPTION

Provided herein are methods and materials that can be used to treat mammals having an autoimmune disease (e.g., previously diagnosed as having an autoimmune disease). For example, provided herein are materials and methods for producing a T cell containing a FOXP3 polypeptide and a microRNA (miRNA) (e.g., any of the exemplary miRNAs described herein). Also provided herein are methods and materials for treating a mammal having an autoimmune disease (e.g., any of the exemplary autoimmune diseases described herein or known in the art), where the methods include administering to the mammal an effective amount of any of the T cells described herein or any of the compositions described herein.

Also provided herein are methods and materials for introducing into a T cell (e.g., CD4³⁰ T cells, CD4⁺CD45RA⁺ T cells, CD4⁺ CD62L⁺, or central memory T cell) a first nucleic acid sequence encoding a FOXP3 polypeptide and a second nucleic acid encoding a microRNA.

In some embodiments, the first nucleic acid sequence encoding the FOXP3 polypeptide can encode one or more fragments of a full length FOXP3 polypeptide (e.g., a full length FOXP3 polypeptide such as version NP_001107849.1). In some embodiments, a cell can be transduced with a first nucleic acid sequence encoding a FOXP3 polypeptide that includes at least the regions of FOXP3 that have DNA-binding properties (e.g., polypeptide fragments of FOXP3 that can bind to a ATAACA DNA sequence) (Li et al., Acta Biochim. Biophysc. Sin., 49(9):792-99 (2017)).

In some embodiments, a second nucleic acid encoding a microRNA (miRNA) is introduced into a T cell (e.g., CD4⁺ T cell, CD4⁺CD45RA⁺ T cell, CD4⁺ CD62L⁺, or central memory T cell) along with the first nucleic acid encoding the FOXP3 polypeptide. In some embodiments, introducing a first nucleic acid encoding a FOXP3 polypeptide and a second nucleic acid encoding a miRNA into CD4⁺T cells enhances the stability and suppressive activity of the T cells. For example, a miRNA can increase the half-life of a FOXP3 mRNA in a mammalian cell as compared to when the miRNA is not present in the mammalian cell. Non-limiting examples of miRNA include miR-142, miR-155, miR-15b, miR-16, miR-146a, miR-21a, miR-99a, miR-150, miR-10a, miR-95, miR-126, miR-29a, miR-24, miR-181c, and miR-101.

For example, a first nucleic acid encoding the FOXP3 polypeptide and a second nucleic acid encoding a miR-142-5p can be introduced into a T cell (e.g., CD4⁺ T cell, CD4⁺CD45RA⁺ T cell, CD4⁺ CD62L⁺ T cell, or central memory T cell). The cAMP-hydrolyzing enzyme, phosphodiesterase-3b (Pde3b), reduces cAMP levels that are needed to maintain or enhance Treg suppressive activity. miR-142-5p has been shown to inhibit the expression of Pde3b, and thus will enhance enforced FOXP3⁺CD4⁺ T cell suppressive activity. Anandagoda, et al., J. Clin. Invest. 2019 Mar 1; 129(3): 1257-1271. In another example, a first nucleic acid encoding the FOXP3 polypeptide and a second nucleic acid encoding a miR-155-5p can be introduced into a T cell (e.g., CD4⁺ T cell, CD4⁺CD45RA⁺ T cell, CD4⁺ CD62L⁺ T cell, or central memory T cell). In some embodiments, a second nucleic acid encoding a miRNA can include a miRNA that inhibits cyclin dependent kinases 8 and 19 (CDK8/19). (See, Han et al., Am. J. Cancer Res.; 7(10): 2081-2090 (2017); and Li et al., J. Transl. Med., 13: 271 (2015)). Examples of miRNA that inhibit CDK8/19 include miR-148, miR-101, and miR-107. Other miRNAs that inhibit these kinases can be identified using software known in the art, including TargetScan.

### FOXP3

As used herein, “FOXP3” refers to the FOXP3 gene or protein that is a transcription factor in the Forkhead box (Fox) family of transcription factors (Sakaguchi et al., Int'l Immun., 21(10):1105-1111 (2009); Pandiyan, et al., Cytokine, 76(1):13-24 (2015)), or a variant thereof (e.g., a FOXP3 protein having one or more (e.g., one, two, three, four, five, six, seven, eight, nine, ten, eleven, twelve, thirteen, fourteen, fifteen, sixteen, seventeen, eighteen, nineteen, or twenty amino acid substitutions, amino acid deletions, or amino acid insertions as compared to a wildtype FOXP3 protein). In some embodiments, when preparing a T cell to be used in the treatment of a mammal having an autoimmune disease by administering to the mammal the T cell, FOXP3 refers to human FOXP3 or a variant thereof. An example of a wildtype human FOXP3 polypeptide includes, without limitation, NCBI reference sequence: NP_054728.2 or a fragment thereof.

In some embodiments referring to a FOXP3 polypeptide, the amino acid sequence of the FOXP3 polypeptide is at least 70% (e.g., at least 75%, 80%, 85%, 90%, 95%, 99% and 100%) identical to:

(SEQ ID NO: 1)
MPNPRPGKPSAPSLALGPSPGASPSWRAAPKASDLLGARGPGGTFQGRDL
RGGAHASSSSLNPMPPSQLQLPTLPLVMVAPSGARLGPLPHLQALLQDRP
HFMHQLSTVDAHARTPVLQVHPLESPAMISLTPPTTATGVFSLKARPGLP
PGINVASLEWVSREPALLCTFPNPSAPRKDSTLSAVPQSSYPLLANGVCK
WPGCEKVFEEPEDFLKHCQADHLLDEKGRAQCLLQREMVQSLEQQLVLEK
EKLSAMQAHLAGKMALTKASSVASSDKGSCCIVAAGSQGPVVPAWSGPRE
APDSLFAVRRHLWGSHGNSTFPEFLHNMDYFKFHNMRPPFTYATLIRWAI
LEAPEKQRTLNEIYHWFTRMFAFFRNHPATWKNAIRHNLSLHKCFVRVES
EKGAVWTVDELEFRKKRSQRPSRCSNPTPGP.

In some embodiments referring to a first nucleic acid sequence encoding a FOXP3 (e.g., full length FOXP3) polypeptide, the nucleic acid sequence is at least 70% (e.g., at least 75%, 80%, 85%, 90%, 95%, 99% and 100%) identical to:

(SEQ ID NO: 2)
ATGCCTAACCCCCGCCCTGGAAAACCATCTGCCCCTTCACTGGCCCTGGG
ACCTTCACCCGGAGCCTCACCATCTTGGAGAGCCGCCCCCAAGGCCAGCG
ACCTGCTGGGAGCCAGAGGCCCCGGCGGCACCTTCCAGGGCAGGGATCTG
CGCGGCGGCGCCCACGCCAGCTCCTCTAGCCTGAACCCCATGCCCCCTTC
TCAGCTCCAGCTGCCCACACTGCCCCTGGTCATGGTGGCACCTAGCGGAG
CAAGGCTGGGACCACTGCCACACCTCCAGGCCCTGCTCCAGGACAGACCT
CACTTTATGCACCAGCTGTCCACCGTGGATGCACACGCAAGGACACCCGT
GCTCCAGGTGCACCCTCTGGAGTCTCCAGCCATGATCAGCCTGACCCCAC
CAACCACAGCAACAGGCGTGTTCTCCCTGAAGGCCAGACCTGGCCTGCCT
CCAGGCATCAACGTGGCCTCCCTGGAGTGGGTGTCTAGGGAGCCAGCCCT
GCTGTGCACCTTTCCTAATCCATCTGCCCCCCGCAAGGACTCCACACTGT
CTGCCGTGCCACAGTCCTCTTACCCCCTGCTGGCCAACGGCGTGTGCAAG
TGGCCTGGCTGTGAGAAGGTGTTCGAGGAGCCAGAGGATTTTCTGAAGCA
CTGCCAGGCCGACCACCTGCTGGATGAGAAGGGAAGGGCACAGTGTCTGC
TCCAGAGGGAGATGGTGCAGAGCCTGGAGCAGCAGCTGGTGCTGGAGAAG
GAGAAGCTGTCCGCCATGCAGGCACACCTGGCAGGCAAGATGGCACTGAC
CAAGGCCAGCTCCGTGGCCTCTAGCGACAAGGGCAGCTGCTGTATCGTGG
CCGCCGGCTCCCAGGGACCAGTGGTGCCCGCCTGGTCTGGACCCAGGGAG
GCACCTGACAGCCTGTTCGCCGTGCGGAGACACCTGTGGGGCAGCCACGG
CAATTCCACCTTCCCCGAGTTTCTGCACAACATGGATTACTTCAAGTTTC
ACAATATGCGGCCCCCTTTTACCTATGCCACACTGATCAGATGGGCCATC
CTGGAGGCCCCAGAGAAGCAGCGCACCCTGAACGAAATCTACCACTGGTT
CACACGGATGTTTGCCTTCTTTAGAAATCACCCCGCCACCTGGAAGAACG
CCATCAGGCACAATCTGTCCCTGCACAAGTGTTTCGTGCGCGTGGAGTCT
GAGAAGGGCGCCGTGTGGACAGTGGATGAGCTGGAGTTCAGAAAGAAGAG
AAGCCAGAGACCATCCAGGTGTTCAAACCCTACCCCAGGACCC.

As used herein, “T-cell function” refers to a T cell's (e.g., any of the exemplary T cells described herein) survival, stability, and/or ability to execute its intended function. For example, a CD4⁺ T cell can have an immunosuppressive function. A CD4⁺ T cell including a nucleic acid encoding a FOXP3 polypeptide can have a FOXP3-dependent expression profile that increases the immunosuppressive function of the T cell. For example, a cell transduced with a mutated FOXP3 polypeptide as described herein can have increased expression of genes that are transcriptional targets of a FOXP3 that can result in increased Treg cell function.

As used herein, the term “activation” refers to induction of a signal on an immune cell (e.g., a B cell or T cell) that to results in initiation of the immune response (e.g., T cell activation). In some cases, upon binding of an antigen to a T cell receptor (TCR, the immune cell can undergo changes in protein expression that result in the activation of the immune response. In some cases, a TCR includes a cytoplasmic signaling sequence that can drive T cell activation.

### microRNAs

As used herein, the term “microRNAs” (miRNAs) refers to short (20-24 nucleotide) non-coding RNAs that are involved in post-transcriptional regulation of gene expression in multicellular organisms by affecting both the stability and translation of mRNAs. miRNAs are transcribed by RNA polymerase II as part of capped and polyadenylated primary transcripts (pri-miRNAs) that can be either protein-coding or non-coding. The primary transcript is cleaved by the Drosha ribonuclease III enzyme to produce an approximately 70-nt stem-loop precursor miRNA (pre-miRNA), which is further cleaved by the cytoplasmic Dicer ribonuclease to generate the mature miRNA and antisense miRNA star (miRNA*) products. The mature miRNA is incorporated into a RNA-induced silencing complex (RISC), which recognizes target mRNAs through imperfect base pairing with the miRNA and most commonly results in translational inhibition or destabilization of the target mRNA.

As used herein, miRNA 142-5p refers to a human miR142 (hsa-miR-142-5p; miRBase Accession No: MIMAT0000433). An example of a mature human miR-142-5p includes a nucleic acid sequence that is at least 70% (e.g., at least 75%, at least 80%, at least 85%, at least 90%, at least 95%, at least 99%, or at least 100%) identical to:

| (SEQ ID NO: 3) | |
|---|---|
| | CAUAAAGUAGAAAGCACUACU. |

An example of a stem-loop precursor miRNA (pre-miRNA) that can be used to generate mature human miR-142 includes a nucleic acid sequence that is at least 70% (e.g., at least 75%, at least 80%, at least 85%, at least 90%, at least 95%, at least 99%, or at least 100%) identical to:

(miRBase Accession No: MI0000458)
(SEQ ID NO: 4)
GACAGUGCAGUCACCCAUAAAGUAGAAAGCACUACUAACAGCACUGGAGG
GUGUAGUGUUUCCUACUUUAUGGAUGAGUGUACUGUG.

In some embodiments, the primary miRNA sequence for miR-142 includes a nucleic acid sequence that is at least 70% (e.g., at least 75%, at least 80%, at least 85%, at least 90%, at least 95%, at least 99%, or at least 100%) identical to:

(SEQ ID NO: 5)
CCTAGTCTCTACCTGAGTGTCTCTGAAACTGGGGGGATGGGGTGGAGCCT
TTAGGGGGAAGGGAAGAGGGAACTGAAGAGGAAGTGGGGGAGGGAGGTAG
AGGAGGCAAGTCTGGCGCCATGCTGAGTCACCGCCCACAAGGCCCAGGGC
GGGCCCTCGGGGGGCCCTGGCAGGGTTGGGGGGATCTTAGGAAGCCACAA
GGAGGGCTGGGGGGCTCTTGGAGCAGGAGTCAGGAGGCCTGGGCAGCCTG
AAGAGTACACGCCGACGGACAGACAGACAGTGCAGTCACCCATAAAGTAG
AAAGCACTACTAACAGCACTGGAGGGTGTAGTGTTTCCTACTTTATGGAT
GAGTGTACTGTGGGCTTCGGAGATCACGCCACTGCTGCCGCCCGCTGCCC
GCCACCATCTTCCTCGGCGCTCGGGGACCTCGTGTGACAGGTGAGCACCT
TACGGCCCCTCCCTACCCTGCCCAGATGCCTGAAAGGCCTCCATGGCTTT
CCTGCCCTTCCTGGTTCCGGACAGCTGGGGAAAGGCCACAGCAGCTCCTC
TGCTGCCCTGCAGTCTTTGGGGGCGGGGAGGGCTGGACATGTGGAACCCT
GATGCAGCCGCAGCGTCAAGGACGAGGAAGGGGTGGGAAGGGATGGTACG
TGGAG.

As used herein, miR-155-5p refers to a human miR-155 (hsa-miR-155-5p; miRBase Accession No: MIMAT0000646). An example of a mature human miR-155-5p includes a nucleic acid sequence that is at least 70% (e.g., at least 75%, at least 80%, at least 85%, at least 90%, at least 95%, at least 99%, or at least 100%) identical to:

| (SEQ ID NO: 6) | |
|---|---|
| | UUAAUGCUAAUCGUGAUAGGGGUU. |

An example of a stem-loop precursor miRNA (pre-miRNA) that can be used to generate mature human miR-155 includes a nucleic acid sequence that is at least 70% (e.g., at least 75%, at least 80%, at least 85%, at least 90%, at least 95%, at least 99%, or at least 100%) identical to:

(miRBase Accession No: MI0000681)
(SEQ ID NO: 7)
CUGUUAAUGCUAAUCGUGAUAGGGGUUUUUGCCUCCAACUGACUCCUACA
UAUUAGCAUUAACAG.

In some embodiments, the primary miRNA sequence for miR-155 includes a nucleic acid sequence that is at least 70% (e.g., at least 75%, at least 80%, at least 85%, at least 90%, at least 95%, at least 99%, or at least 100%) identical to:

(SEQ ID NO: 8)
TATGCCTAAAGGTAACAATGTCATCTTTTAATTGCCAATTTCTCTACCAC
TTTCAAAAAATTACTTCCAAGGATTTAATGAGCTCCTTCCTTTCAACAGA
AAATGGACTATTTTCCTTTCAGATTTACTATATGCTGTCACTCCAGCTTT
ATAACCGCATGTGCATACACAAACATTTCTTTCTCTCTTGCAGGTGGCAC
AAACCAGGAAGGGGAAATCTGTGGTTTAAATTCTTTATGCCTCATCCTCT
GAGTGCTGAAGGCTTGCTGTAGGCTGTATGCTGTTAATGCTAATCGTGAT
AGGGGTTTTTGCCTCCAACTGACTCCTACATATTAGCATTAACAGTGTAT
GATGCCTGTTACTAGCATTCACATGGAACAAATTGCTGCCGTGGGAGGAT
GACAAAGAAGCATGAGTCACCCTGCTGGATAAACTTAGACTTCAGGCTTT
ATCATTTTTCAATCTGTTAATCATAATCTGGTCACTGGGATGTTCAACCT
TAAACTAAGTTTTGAAAGTAAGGTTATTTAAAAGATTTATCAGTAGTATC
CTAAATGCAAACATTTTCATTTAAATGTCAAGCCCATGTTTGTTTTTATC
ATTAACAGAAAATATATTCATGTCATTCTTAATTGCAGGTTTTG.

As used herein, miR-15b-5p refers to a human miR-15b (hsa-miR-15b-5p; miRBase Accession No: MIMAT0000417). An example of a mature human miR-15b-5p includes a nucleic acid sequence that is at least 70% (e.g., at least 75%, at least 80%, at least 85%, at least 90%, at least 95%, at least 99%, or at least 100%) identical to:

| (SEQ ID NO: 9) | |
|---|---|
| | UAGCAGCACAUCAUGGUUUACA. |

An example of a stem-loop precursor miRNA (pre-miRNA) that can be used to generate mature human miR-15b includes a nucleic acid sequence that is at least 70% (e.g., at least 75%, at least 80%, at least 85%, at least 90%, at least 95%, at least 99%, or at least 100%) identical to:

(miRBaseAccession No: MI0000438)
(SEQ ID NO: 10)
UUGAGGCCUUAAAGUACUGUAGCAGCACAUCAUGGUUUACAUGCUACAGU
CAAGAUGCGAAUCAUUAUUUGCUGCUCUAGAAAUUUAAGGAAAUUCAU.

In some embodiments, the primary miRNA sequence for miR-15b includes a nucleic acid sequence that is at least 70% (e.g., at least 75%, at least 80%, at least 85%, at least 90%, at least 95%, at least 99%, or at least 100%) identical to:

(SEQ ID NO: 11)
TTTTCCTCAAAACAGGAAGGGGATGATTATGAAGTCATTCCTAACAGTAA
TTTCTATGTATCCAGAACGGCCTGCAGAGATAATACTTCTGTCTATCACA
TAAGTGGAAAGAAAAAGACATTTAAGGATGTTGGAAATCTTCTTCGAAGC
CATGGAATTGACTTGGACCATAATAGATTTTTAATTTTACAGGTAAGTTT
ATTAAAGACTTCAAAGATTCTCTTATTCTTGTTACTTTTTTTTCTATAAA
GCTAGGTTGGATGAATCCTACATTTTTGAGGCCTTAAAGTACTGTAGCAG
CACATCATGGTTTACATGCTACAGTCAAGATGCGAATCATTATTTGCTGC
TCTAGAAATTTAAGGAAATTCATTCAAAACTATGTTTTCATCATCAGATG
TTCGTTTTATGTTTGGATGAACTGACATACTTGTTCCACTCTAGCAGCAC
GTAAATATTGGCGTAGTGAAATATATATTAAACACCAATATTACTGTGCT
GCTTTAGTGTGACAGGGATACAGCAACTATTTTATCAATTGTTTGTATTT
CCCTTTAAGGTAACATTTTAAATGAAATGTATTATATTTTAATCTATCCT
TTTCCTTTGTTTTTGTTCTTATTATCTCTTCTGATATATAACCAAAAAAT
GAA.

As used herein, miR-146a-5p refers to a human miR-146a (hsa-miR-146a-5p; miRBase Accession No: MIMAT0000449). An example of a mature human miR-146a-5p includes a nucleic acid sequence that is at least 70% (e.g., at least 75%, at least 80%, at least 85%, at least 90%, at least 95%, at least 99%, or at least 100%) identical to:

| (SEQ ID NO: 12) | |
|---|---|
| | UGAGAACUGAAUUCCAUGGGUU. |

An example of a stem-loop precursor miRNA (pre-miRNA) that can be used to generate mature human miR-146a includes a nucleic acid sequence that is at least 70% (e.g., at least 75%, at least 80%, at least 85%, at least 90%, at least 95%, at least 99%, or at least 100%) identical to:

(miRBase Accession No: MI0000477)
(SEQ ID NO: 13)
CCGAUGUGUAUCCUCAGCUUUGAGAACUGAAUUCCAUGGGUUGUGUCAGU
GUCAGACCUCUGAAAUUCAGUUCUUCAGCUGGGAUAUCUCUGUCAUCGU.

In some embodiments, the primary miRNA sequence for miR-146a includes a nucleic acid sequence that is at least 70% (e.g., at least 75%, at least 80%, at least 85%, at least 90%, at least 95%, at least 99%, or at least 100%) identical to:

(SEQ ID NO: 14)
TCAAGCGATCCTCCCACCACAGGCCATCATGCATGGCTCATTTTTTATTT
TTAGTAGAGACAAATTCTCCATGTTGCCCAGGCTAGTCCTGAACTCCTGG
GCTCAAGAGATCCACCCACATCAGCCTTCCAGACTGCTGGCCTGGTCTCC
TCCAGATGTTTATAACTCATGAGTGCCAGGACTAGACCTGGTACTAGGAA
GCAGCTGCATTGGATTTACCAGGCTTTTCACTCTTGTATTTTACAGGGCT
GGGACAGGCCTGGACTGCAAGGAGGGGTCTTTGCACCATCTCTGAAAAGC
CGATGTGTATCCTCAGCTTTGAGAACTGAATTCCATGGGTTGTGTCAGTG
TCAGACCTCTGAAATTCAGTTCTTCAGCTGGGATATCTCTGTCATCGTGG
GCTTGAGGACCTGGAGAGAGTAGATCCTGAAGAACTTTTTCAGTCTGCTG
AAGAGCTTGGAAGACTGGAGACAGAAGGCAGAGTCTCAGGCTCTGAAGGT
ATAAGGAGTGTGAGTTCCTGTGAGAAACACTCATTTGATTGTGAAAAGAC
TTGAATTCTATGCTAAGCAGGGTTCCAAGTAGCTAAATGAATGATCTCAG
CAAGTCTCTCTTGCTGCTGCTGCTACTCGTTTACATTTATTGATTACTTA
CGATGATTCAGGTACTGTTGTAAGTGCTTTACATG.

As used herein, miR-146b-5p refers to a human miR-146b (hsa-miR-146b-5p; miRBase Accession No: MIMAT0002809). An example of a mature human miR-146b-5p includes a nucleic acid sequence that is at least 70% (e.g., at least 75%, at least 80%, at least 85%, at least 90%, at least 95%, at least 99%, or at least 100%) identical to:

| (SEQ ID NO: 15) | |
|---|---|
| | UGAGAACUGAAUUCCAUAGGCUG. |

An example of a stem-loop precursor miRNA (pre-miRNA) that can be used to generate mature human miR-146b includes a nucleic acid sequence that is at least 70% (e.g., at least 75%, at least 80%, at least 85%, at least 90%, at least 95%, at least 99%, or at least 100%) identical to:

(miRBase Accession No: MI0003129)
(SEQ ID NO: 16)
CCUGGCACUGAGAACUGAAUUCCAUAGGCUGUGAGCUCUAGCAAUGCCCU
GUGGACUCAGUUCUGGUGCCCGG.

As used herein, miR-21a-5p refers to a human miR-21a (hsa-miR-21a-5p; miRBase Accession No: MIMAT0000530). An example of a mature human miR-21a-5p includes a nucleic acid sequence that is at least 70% (e.g., at least 75%, at least 80%, at least 85%, at least 90%, at least 95%, at least 99%, or at least 100%) identical to: UAGCUUAUCAGACUGAUGUUGA (SEQ ID NO: 17). An example of a stem-loop precursor miRNA (pre-miRNA) that can be used to generate mature human miR-21a includes a nucleic acid sequence that is at least 70% (e.g., at least 75%, at least 80%, at least 85%, at least 90%, at least 95%, at least 99%, or at least 100%) identical to:

(miRBase Accession No: MI0000569)
(SEQ ID NO: 18)
UGUACCACCUUGUCGGAUAGCUUAUCAGACUGAUGUUGACUGUUGAAUCU
CAUGGCAACAGCAGUCGAUGGGCUGUCUGACAUUUUGGUAUC.

In some embodiments, the primary miRNA sequence for miR-21a includes a nucleic acid sequence that is at least 70% (e.g., at least 75%, at least 80%, at least 85%, at least 90%, at least 95%, at least 99%, or at least 100%) identical to:

(SEQ ID NO: 19)
CAGTTTTCTTGCCGTTCTGTAAGTGTTTTATTCTTAGTGTGATTTTTTTC
CATTGGGATGTTTTTGATTGAACTTGTTCATTTTGTTTTGCTTGGGAGGA
AAATAAACAATTTTACTTTTTTCCTTTAGGAGCATTATGAGCATTATGTC
AGAATAGAATAGAATTGGGGTTCGATCTTAACAGGCCAGAAATGCCTGGG
TTTTTTTGGTTTGTTTTTGTTTTTGTTTTTTTATCAAATCCTGCCTGACT
GTCTGCTTGTTTTGCCTACCATCGTGACATCTCCATGGCTGTACCACCTT
GTCGGGTAGCTTATCAGACTGATGTTGACTGTTGAATCTCATGGCAACAC
CAGTCGATGGGCTGTCTGACATTTTGGTATCTTTCATCTGACCATCCATA
TCCAATGTTCTCATTTAAACATTACCCAGCATCATTGTTTATAATCAGAA
ACTCTGGTCCTTCTGTCTGGTGGCACTTAGAGTCTTTTGTGCCATAATGC
AGCAGTATGGAGGGAGGATTTTATGGAGAAATGGGGATAGTCTTCATGAC
CACAAATAAATAAAGGAAAACTAAGCTGCATTGTGGGTTTTGAAAAGGTT
ATTATACTTCTTAACAATTCTTTTTTTCAGGGACTTTTCTAGCTGTATGA
C.

As used herein, miR-99a-5p refers to a human miR-99a (hsa-miR-99a-5p; miRBase Accession No: MIMAT0000097). An example of a mature human miR-99a-5p includes a nucleic acid sequence that is at least 70% (e.g., at least 75%, at least 80%, at least 85%, at least 90%, at least 95%, at least 99%, or at least 100%) identical to:

| (SEQ ID NO: 20) | |
|---|---|
| | AACCCGUAGAUCCGAUCUUGUG. |

An example of a stem-loop precursor miRNA (pre-miRNA) that can be used to generate mature human miR-99a includes a nucleic acid sequence that is at least 70% (e.g., at least 75%, at least 80%, at least 85%, at least 90%, at least 95%, at least 99%, or at least 100%) identical to:

(miRBase Accession No: MIMAT0000451)
(SEQ ID NO: 21)
CCCAUUGGCAUAAACCCGUAGAUCCGAUCUUGUGGUGAAGUGGACCGCAC
AAGCUCGCUUCUAUGGGUCUGUGUCAGUGUG.

In some embodiments, the primary miRNA sequence for miR-99a includes a nucleic acid sequence that is at least 70% (e.g., at least 75%, at least 80%, at least 85%, at least 90%, at least 95%, at least 99%, or at least 100%) identical to:

(SEQ ID NO: 22)
TGTATGGATTCTTTTTTCTTTTAAAACTCAATTAGAATAGTTTAATTCCA
AAATATTTACTATTGAAACAAAAGCAGTTCGTGAAAAATTTTTCAATAAA
CTACTTTTTAAATTCTCATGCATATAAATTTGTATTTAGTTTTGAATATT
TATGAAGGCCTTTAATGGAGAATATGCATCCTTAGAACTCAGCATTTAAA
ACATTTATACTAAAGGATCAATATTATTTTTGACTCTTAATTGCATCAGA
TATTTACAACAAATTCTATATTAATAGGGGGCCCATGCAAGATGTTGCCC
ATTGGCATAAACCCGTAGATCCGATCTTGTGGTGAAGTGGACCGCACAAG
CTCGCTTCTATGGGTCTGTGTCAGTGTGGTAATCTGACAAAATGCTATAC
ACAGTGCCGTTCAACAATAGTTCAGTAAAATCCTGTTAAACTCCAGTTGA
TTATATACTTTTGAAGTCATTATATTTTCCTTTGTTTTTAATGTTTATTT
TAATCATTGTCTGTCTTACAAGGCAGGCTTCAATTCTCAACAACTTGGAA
GCGTTTATATCACACCCATTCAAGTTCGATTCCATGTACAGTAAATTGCA
TAAGAAAGTTGAACCTTTATAGCAGGGTTTGGACCAGGATCTGAATAGAT
TCTGTTCAGAAACTTCAGTGC.

As used herein, miR-150-5p refers to a human miR-150 (hsa-miR-150-5p; miRBase Accession No: MIMAT0000451). An example of a mature human miR-150-5p includes a nucleic acid sequence that is at least 70% (e.g., at least 75%, at least 80%, at least 85%, at least 90%, at least 95%, at least 99%, or at least 100%) identical to:

| (SEQ ID NO: 23) | |
|---|---|
| | UCUCCCAACCCUUGUACCAGUG. |

miRNA (pre-miRNA) that can be used to generate mature human miR-150 includes a nucleic acid sequence that is at least 70% (e.g., at least 75%, at least 80%, at least 85%, at least 90%, at least 95%, at least 99%, or at least 100%) identical to:

(miRBase Accession No: MI0000479)
(SEQ ID NO: 24)
CUCCCCAUGGCCCUGUCUCCCAACCCUUGUACCAGUGCUGGGCUCAGACC
CUGGUACAGGCCUGGGGGACAGGGACCUGGGGAC.

In some embodiments, the primary miRNA sequence for miR-150 includes a nucleic acid sequence that is at least 70% (e.g., at least 75%, at least 80%, at least 85%, at least 90%, at least 95%, at least 99%, or at least 100%) identical to:

(SEQ ID NO: 25)
CGCAACCTCCTATTCCCCTCTGGGTCTCCGTCCCCTCCCTCTGGAGTCCA
CACTCCCTCTTTGCCCCTTGCTGGTTCTCTACTGCCCCCAGCATAGGGTG
GAGTGGGTGTGCAGTTTCTGCGACTCAGGGTGGCGTCCCCCCAACCTGTC
CCTGCCCCTTCCTGCCCTCTTTGATGCGGCCCCACTTCCTCTGGCAGGAA
CCCCCGCCCTCCCTGGACCTGGGTATAAGGCAGGGACTGGGCCCACGGGG
AGGCAGCGTCCCCGAGGCAGCAGCGGCAGCGGCGGCTCCTCTCCCCATGG
CCCTGTCTCCCAACCCTTGTACCAGTGCTGGGCTCAGACCCTGGTACAGG
CCTGGGGGACAGGGACCTGGGGACCCCGGCACCGGCAGGCCCCAAGGGGT
GAGGTGAGCGGGCATTGGGACCTCCCCTCCCTGTACTCCCATCTCTGCTG
CGGCTTTTATGCGTCTCTCCCCTTCGGGTCCCACATATCCTCTGGTGCGC
TCCTGCCTCACCGCCCCCACCCCATGCCTGTCGTCCCCACCTCTGTGTGA
TGCGCAAAGTACACCTGTTTCTATTGTACCTGCCTCTCGCGGTGGTCTGT
GCTCTCCCCAGCTCTGCAAAACCCCTCCTCCCCATGTGCCACAACCCTGG
GCCACCGTGTGTCCTGTCCTGTT.

As used herein, miR-10a-5p refers to a human miR-10a (hsa-miR-10a-5p; miRBase Accession No: MIMAT0000253). An example of a mature human miR-10a-5p includes a nucleic acid sequence that is at least 70% (e.g., at least 75%, at least 80%, at least 85%, at least 90%, at least 95%, at least 99%, or at least 100%) identical to:

| (SEQ ID NO: 26) | |
|---|---|
| | UACCCUGUAGAUCCGAAUUUGUG. |

An example of a stem-loop precursor miRNA (pre-miRNA) that can be used to generate mature human miR-10a includes a nucleic acid sequence that is at least 70% (e.g., at least 75%, at least 80%, at least 85%, at least 90%, at least 95%, at least 99%, or at least 100%) identical to:

(miRBase Accession No: MI0000266)
(SEQ ID NO: 27)
GAUCUGUCUGUCUUCUGUAUAUACCCUGUAGAUCCGAAUUUGUGUAAGGA
AUUUUGUGGUCACAAAUUCGUAUCUAGGGGAAUAUGUAGUUGACAUAAAC
ACUCCGCUCU.

In some embodiments, the primary miRNA sequence for miR-10a includes a nucleic acid sequence that is at least 70% (e.g., at least 75%, at least 80%, at least 85%, at least 90%, at least 95%, at least 99%, or at least 100%) identical to:

(SEQ ID NO: 28)
CCCTTCCTCCTTTTGTGCTTAGCTAATGTTTACATCTCATAATTCATGCG
CCACCGAGAGTTGCGCGGCGGCGGCGGAGGCAAGGTTCTCGTCCCTTTGC
GAACTGGCTACTTGAAATTCTAATAGAAGAGGAGAATTGAAAACCTTGTA
ATCCCAAGAACAGACTCGCACTGCCTTTTTCTGTTCCCAGAGCTCAAAAC
TAGAACAAAACGAAATAAAACCAAAGCACTCAAACCACACCCCAAACGAA
GAAGGCGCGGAAAGTAGGAGAACTGGAAAATTTCTGGGCCAAGAAGATCT
GTCTGTCTTCTGTATATACCCTGTAGATCCGAATTTGTGTAAGGAATTTT
GTGGTCACAAATTCGTATCTAGGGGAATATGTAGTTGACATAAACACTCC
GCTCTTATTTTTCCAGAAGAAAAAAATATATATATATGTATATGTAGTAT
TTTTCTGAATGAGGACAGTCTGGTGACTGGCCACACGAAGACTCCTTCCT
CTTTATTCCTCTATCTTTTCCTCCTTCAACTGGTTAGAGATGGAGAAATC
ATCTACCTGAACTCTCCCCACGCCTGCCCCTGGGAGCCTTGGTCCCTTTT
CACATCTCTTTAAGAGGTTAATGTTATTGTTGTTGAAGTTTTAAATTTAT
CTTCTCGTCCCAAACGCACCCATTTTCTATTCTGGGCTCAGGGAGGCTTT
AT.

As used herein, miR-95-5p refers to a human miR-95 (hsa-miR-95-5p; miRBase Accession No: MIMAT0026473). An example of a mature human miR-95-5p includes a nucleic acid sequence that is at least 70% (e.g., at least 75%, at least 80%, at least 85%, at least 90%, at least 95%, at least 99%, or at least 100%) identical to:

(SEQ ID NO: 29)
UCAAUAAAUGUCUGUUGAAUU.

An example of a stem-loop precursor miRNA (pre-miRNA) that can be used to generate mature human miR-95 includes a nucleic acid sequence that is at least 70% (e.g., at least 75%, at least 80%, at least 85%, at least 90%, at least 95%, at least 99%, or at least 100%) identical to:

(SEQ ID NO: 30)
AACACAGUGGGCACUCAAUAAAUGUCUGUUGAAUUGAAAUGCGUUACAUU
CAACGGGUAUUUAUUGAGCACCCACUCUGUG (miRBase Accession
No: MI0000097).

In some embodiments, the primary miRNA sequence for miR-95 includes a nucleic acid sequence that is at least 70% (e.g., at least 75%, at least 80%, at least 85%, at least 90%, at least 95%, at least 99%, or at least 100%) identical to:

(SEQ ID NO: 31)
TGTGCCTGGATCAGGTGAGCCTTGCCGTCTGCGGCCTCCTGAGGAATCCA
GGCGGGAAGGGGAGCACATGGAGATTGAGAGGGAGCCACCCTTTTTTTTC
TCTCGGGGTTCTTTTGATTGTCACCAAGTAGCCCCCAGACCTGAGTCCGT
GGGCACCAGTTCCATTTGCACACAGCTTTTACTGAACAAAGCATTTGCAC
ACAGCAAGGCACGCCACCTGCACCCCGGGACGTCCATCTGTAGCGCGCCC
AAGGAAGGTAGGATTGTGACACCCAACACAGTGGGCACTCAATAAATGTC
TGTTGAATTGAAATGCGTTACATTCAACGGGTATTTATTGAGCACCCACT
CTGTGCCAGACGCTGAGCGGGGCGCCGAGGGGGACAGAGAAGACAAGAGC
AGCCCCTGACCTGGAGAAGCGTGCAGGGAGCTGAGAGAGGCAGAGACGCC
GACAGAGCGAGCGCACACGCATCCCGCCGCCGCCTGCCCGCCGGGCACCT
CTTTGGCTTCGCAAGGGCCCGATCACAAACTCATCTCACCCACAACACCA
TCTGCACATCTCACCCGGGACTGAGTTCATCATTCAGTCATTCATCCATC
CCTCCACCAGATACTTACTGAGCACCTACTATGTGCTGGACATGG.

As used herein, miR-126-5p refers to a human miR-126 (hsa-miR-126-5p; miRBase Accession No: MIMAT0000444). An example of a mature human miR-126-5p includes a nucleic acid sequence that is at least 70% (e.g., at least 75%, at least 80%, at least 85%, at least 90%, at least 95%, at least 99%, or at least 100%) identical to:

(SEQ ID NO: 32)
CAUUAUUACUUUUGGUACGCG.

An example of a stem-loop precursor miRNA (pre-miRNA) that can be used to generate mature human miR-126 includes a nucleic acid sequence that is at least 70% (e.g., at least 75%, at least 80%, at least 85%, at least 90%, at least 95%, at least 99%, or at least 100%) identical to:

(SEQ ID NO: 33)
GCUGGCGACGGGACAUUAUUACUUUUGGUACGCGCUGUGACACUUCAAAC
UCGUACCGUGAGUAAUAAUGCGCCGUCCACGGCA (miRBase
Accession No: MI0000471).

In some embodiments, the primary miRNA sequence for miR-126 includes a nucleic acid sequence that is at least 70% (e.g., at least 75%, at least 80%, at least 85%, at least 90%, at least 95%, at least 99%, or at least 100%) identical to:

(SEQ ID NO: 34)
CCAACCCGACAGGTAAACAGCCCTGGCTGTGCCTGGCCTGGGGAGGCGGG
CAGGCAGTGGACATTGCCGTGTGGCTGTTAGGCATGGTGGGGGGCACTGG
AATCTGGGCGGAAGGCGGTGGGGACTCCCTCTCCAGGGAGGGAGGATGGG
GAGGGAGGATAGGTGGGTTCCCGAGAACTGGGGGCAGGTTGCCCGGAGCC
TCATATCAGCCAAGAAGGCAGAAGTGCCCCGTCCCGGGGTCCTGTCTGCA
TCCAGCGCAGCATTCTGGAAGACGCCACGCCTCCGCTGGCGACGGGACAT
TATTACTTTTGGTACGCGCTGTGACACTTCAAACTCGTACCGTGAGTAAT
AATGCGCCGTCCACGGCACCGCATCGAAAACGCCGCTGAGACCTCAGCCT
TGACCTCCCTCAGCGTGGCCGGGACCCTGAGCCTCTGCGCAGAGCCACCC
GCCCCGACGTACTTAGGCGGCATAGCCCTGAGACCTCTGGCCAGCGCCAG
GCAGGCAGCGGGGGCGGCAGAGGCCTGGGCCTGAGTCTTCTGGCTCTGCC
TCTCCCTGGGGACAGGAGGGAGCCTGGGGGTGTGGGTGGGGAGCCGGCCG
GCCGTGACCCAGCGCCTGGCTCTGCCCGCAGGAGTGGACAGTGCAATGAA
GG.

As used herein, miR-29a-5p refers to a human miR-29a (hsa-miR-29a-5p; miRBase Accession No: MIMAT0004503). An example of a mature human miR-29a-5p includes a nucleic acid sequence that is at least 70% (e.g., at least 75%, at least 80%, at least 85%, at least 90%, at least 95%, at least 99%, or at least 100%) identical to:

(SEQ ID NO: 35)
ACUGAUUUCUUUUGGUGUUCAG.

An example of a stem-loop precursor miRNA (pre-miRNA) that can be used to generate mature human miR-29a includes a nucleic acid sequence that is at least 70% (e.g., at least 75%, at least 80%, at least 85%, at least 90%, at least 95%, at least 99%, or at least 100%) identical to:

(SEQ ID NO: 36)
AUGACUGAUUUCUUUUGGUGUUCAGAGUCAAUAUAAUUUUCUAGCACCAU
CUGAAAUCGGUUAU (miRBase Accession No: MI0000087).

In some embodiments, the primary miRNA sequence for miR-29a includes a nucleic acid sequence that is at least 70% (e.g., at least 75%, at least 80%, at least 85%, at least 90%, at least 95%, at least 99%, or at least 100%) identical to:

(SEQ ID NO: 37)
ATACTACACCATTTTCTATCAGAGACTTGAGCATCTGTGGATTTTGGTAT
CCAAGGGGCTTTCTGGAACCAATCCCTCAAGGATACCAAGGGATGAATGT
AATTGTACAGGATATCGCATTGTTGGAATTTTATACTTCTTTGTGGAATA
AACCTATAGCACTTAATAGATAGTACAGACTCATTCCATTGTGCCTGGGT
TAAAGAGCCCAATGTATGCTGGATTTAGTAAGATTTGGGCCCTCCCAACC
CTCACGACCTTCTGTGACCCCTTAGAGGATGACTGATTTCTTTTGGTGTT
CAGAGTCAATATAATTTTCTAGCACCATCTGAAATCGGTTATAATGATTG
GGGAAGAGCACCATGATGCTGACTGCTGAGAGGAAATGTATTGGTGACCG
TTGGGGCCATGGACAAGAACTAAGAAAACAAATGCAAAGCAATAATGCAA
AGGTGATTTTTCTTCTTCCAGTTTCTAAGTTGAATTTCACTGACCTGAAT
TGCATGTGGTATAATACTAACAAATGGTTCACTATTAGCATATCATGAAT
GGTTATACTTTATAGAAATTCCATAGACTTGGTGGGGGTTTTGTTTTGGT
GACGGATACCTAGAAACACTCCTGG.

As used herein, miR-24-2-5p refers to a human miR-24-2 (hsa-miR-24-2-5p; miRBase Accession No: MIMAT0004497). An example of a mature human miR-24-2-5p includes a nucleic acid sequence that is at least 70% (e.g., at least 75%, at least 80%, at least 85%, at least 90%, at least 95%, at least 99%, or at least 100%) identical to:

(SEQ ID NO: 38)
CAUAAAGUAGAAAGCACUACU.

An example of a stem-loop precursor miRNA (pre-miRNA) that can be used to generate mature human miR-24-2 includes a nucleic acid sequence that is at least 70% (e.g., at least 75%, at least 80%, at least 85%, at least 90%, at least 95%, at least 99%, or at least 100%) identical to:

(SEQ ID NO: 39)
CUCUGCCUCCCGUGCCUACUGAGCUGAAACACAGUUGGUUUGUGUACACU
GGCUCAGUUCAGCAGGAACAGGG (miRBase Accession No:
MI0000081).

In some embodiments, the primary miRNA sequence for miR-24 includes a nucleic acid sequence that is at least 70% (e.g., at least 75%, at least 80%, at least 85%, at least 90%, at least 95%, at least 99%, or at least 100%) identical to:

(SEQ ID NO: 40)
GCACTAATCGGACATCTGTCTGAAAGGTCAAATGTATTGAAAGTTGCAAA
AATTCTTCTTACAAAAAACTAAAACCAAATGCATCACCTAAGTCGTGTGA
AATCATGTGGTAGCTCATGGCTGTGAGCGGGGCGGGGCGGGGCTTTCGGA
GGAGCTCCTGTTGTTCTGGGCGCGGTGAACTCTCTCTTGTATTTGCAGTC
CAGGCCTTCGCGTCTCCTGCGCCAGCAGACGGTGCCCACGGAGCTCCCAG
CTGAGGCGCTGCTTCTCCGGGCTGTCGATTGGACCCGCCCTCCGGTGCCT
ACTGAGCTGATATCAGTTCTCATTTTACACACTGGCTCAGTTCAGCAGGA
ACAGGAGTCGAGCCCTTGAGCAAAAAGCCTTCGTGTCTGTAAGTGCCCGA
GGCTCAGGAGAGCTGGGGCTCCCACTCGCGGCAGACAGGCCCGCGTCCAC
CCTGCGTCCACCCCGGCCCGGCGGCAGCACGGTGCCAGTCATCTGCATGT
GCTCCTGCGGCGTGGGGGTTTGTAGACTTGGAAAACCCTGTTGGCAGAAA
GTTAAGAGCTCCCAGGCCTGAAGGCAGGACACAGTGCCAGCAGGGGGCCG
TTTGCCCCCATGTGTAAGGGAGGCAGGGCCCAGCTCTCCCACGGCGAGGT
GA.

As used herein, miR-101-5p refers to a human miR-101 (hsa-miR-101-5p; miRBase Accession No: MIMAT0004513). An example of a mature human miR-101-5p includes a nucleic acid sequence that is at least 70% (e.g., at least 75%, at least 80%, at least 85%, at least 90%, at least 95%, at least 99%, or at least 100%) identical to:

(SEQ ID NO: 41)
CAGUUAUCACAGUGCUGAUGCU.

An example of a stem-loop precursor miRNA (pre-miRNA) that can be used to generate mature human miR-101 includes a nucleic acid sequence that is at least 70% (e.g., at least 75%, at least 80%, at least 85%, at least 90%, at least 95%, at least 99%, or at least 100%) identical to:

(SEQ ID NO: 42)
UGCCCUGGCUCAGUUAUCACAGUGCUGAUGCUGUCUAUUCUAAAGGUACA
GUACUGUGAUAACUGAAGGAUGGCA (miRBase Accession No:
1MI0000103).

In some embodiments, the primary miRNA sequence for miR-101 includes a nucleic acid sequence that is at least 70% (e.g., at least 75%, at least 80%, at least 85%, at least 90%, at least 95%, at least 99%, or at least 100%) identical to:

(SEQ ID NO: 43)
TGCTGGAAGCTTACTGCATATTTGATGTATTAGAGTGAAAACCTAATCAT
GCAGTTGTTCATCCTCATTAATATGGATAAGTCATGTGTTCATCTTTCAT
TCTAATTTAATTCAACTGGGCCTTTTAATATTTCAGCCTCACCACTTGCT
GGGCTCTGATCCTTCTTTTTCTTCTGCCTCCTCACGTCTCCAACCAGAAG
GTGATCTTTTAGTCCTTCACTTCATGGGGAGCCTTCAGAGAGAGTAATGC
AGCCACCAGAAAGGATGCCGTTGACCGACACAGTGACTGACAGGCTGCCC
TGGCTCAGTTATCACAGTGCTGATGCTGTCTATTCTAAAGGTACAGTACT
GTGATAACTGAAGGATGGCAGCCATCTTACCTTCCATCAGAGGAGCCTCA
CCGTACCCAGGAAGAAAGAAGGTGAAAGAGGAATGTGAAACAGGTGGCTG
GGACCCAGAAACCCTCTTACCCTGCACCTCTGTCATACTTCTCCCGGGGC
ATAGGGAGAGTTATTCTGCTTCTCTTTGCCTTGTTTTGTAACATGGGGTA
GTTGTTGGTGCAGCCATGTTGTGCTGAGTGAACATATATTAAGATCTTTG
GAACCTTTAGGAGACTGAAAATAGGTAAGTATGAATTAGTATTTCTGGAA
TGGTATTCAGAGAACTTCG.

As used herein, miR-16 refers to a human miR-16-1 (hsa-miR-16-1-5p; miRBase Accession No: MIMAT0000069). An example of a mature human miR-181c-5p includes a nucleic acid sequence that is at least 70% (e.g., at least 75%, at least 80%, at least 85%, at least 90%, at least 95%, at least 99%, or at least 100%) identical to:

(SEQ ID NO: 44)
UAGCAGCACGUAAAUAUUGGCG.

An example of a stem-loop precursor miRNA (pre-miRNA) that can be used to generate mature human miR-16-1 includes a nucleic acid sequence that is at least 70% (e.g., at least 75%, at least 80%, at least 85%, at least 90%, at least 95%, at least 99%, or at least 100%) identical to:

(SEQ ID NO: 45)
GUCAGCAGUGCCUUAGCAGCACGUAAAUAUUGGCGUUAAGAUUCUAAAAU
UAUCUCCAGUAUUAACUGUGCUGCUGAAGUAAGGUUGAC (miRBase
Accession No: MI0000070).

In some embodiments, the primary miRNA sequence for miR-16 includes a nucleic acid sequence that is at least 70% (e.g., at least 75%, at least 80%, at least 85%, at least 90%, at least 95%, at least 99%, or at least 100%) identical to:

(SEQ ID NO: 46)
ATCTCCTTAAAAATAATTATGCATATTACATCAATGTTATAATGTTTAAA
CATAGATTTTTTTACATGCATTCTTTTTTTCCTGAAAGAAAATATTTTTT
ATATTCTTTAGGCGCGAATGTGTGTTTAAAAAAAATAAAACCTTGGAGTA
AAGTAGCAGCACATAATGGTTTGTGGATTTTGAAAAGGTGCAGGCCATAT
TGTGCTGCCTCAAAAATACAAGGATCTGATCTTCTGAAGAAAATATATTT
CTTTTTATTCATAGCTCTTATGATAGCAATGTCAGCAGTGCCTTAGCAGC
ACGTAAATATTGGCGTTAAGATTCTAAAATTATCTCCAGTATTAACTGTG
CTGCTGAAGTAAGGTTGACCATACTCTACAGTTGTGTTTTAATGTATATT
AATGTTACTAATGTGTTTTCAGTTTTATTGATAGTCTTTTCAGTATTATT
GATAATCTTGTTATTTTTAGTATGATTCTGTAAAAATGAATTAATACTAA
TTTTTCAGATGTATCATCTCTTAAAATACTGTAATTGCAATTTAATAATT
GTATTGAATGCCATCAAGTTTTTTTAAAAAGCTTATGCAGCATTAGAGGA
ATTTATTTTAATGCACATTTATATTCAACATAGACATTAATTCAGATTTT
TACTTGGGA.

As used herein, miR-181c refers to a human miR-181c (hsa-miR-181c-5p; miRBase Accession No: MIMAT0000258). An example of a mature human miR-181c-5p includes a nucleic acid sequence that is at least 70% (e.g., at least 75%, at least 80%, at least 85%, at least 90%, at least 95%, at least 99%, or at least 100%) identical to:

(SEQ ID NO: 47)
AACAUUCAACCUGUCGGUGAGU.

An example of a stem-loop precursor miRNA (pre-miRNA) that can be used to generate mature human miR-181c includes a nucleic acid sequence that is at least 70% (e.g., at least 75%, at least 80%, at least 85%, at least 90%, at least 95%, at least 99%, or at least 100%) identical to:

(SEQ ID NO: 48)
CGGAAAAUUUGCCAAGGGUUUGGGGGAACAUUCAACCUGUCGGUGAGUUU
GGGCAGCUCAGGCAAACCAUCGACCGUUGAGUGGACCCUGAGGCCUGGAA
UUGCCAUCCU (miRBase Accession No: MI0000271).

In some embodiments, the primary miRNA sequence for miR-181c includes a nucleic acid sequence that is at least 70% (e.g., at least 75%, at least 80%, at least 85%, at least 90%, at least 95%, at least 99%, or at least 100%) identical to:

(SEQ ID NO: 49)
GGTAATCTGCACCTCCAGGATCACTTTGTGAATTACTTAAGGAGCGGGCT
TGAGGCCAGCACTCCCCTGCACTGCTACATCTCCATCCCCATAGCAAAGG
CTACATTTTATTTACTAGCATTTATATTTGCCTTGTGTTTTTCCCAGATC
TAGGTGCAAACAGCACCTGAAAAGCGTTTGTTTATTTCATGAGAGAAAAG
GGGTTCCTTACTCTCTCCTCCTCTCCCTCTTCATGCTCTCTGGTTCCCTG
CCACCTACCCCACCCCCGACTCCAGGTCCCGGAAAATTTGCCAAGGGTTT
GGGGGAACATTCAACCTGTCGGTGAGTTTGGGCAGCTCAGGCAAACCATC
GACCGTTGAGTGGACCCTGAGGCCTGGAATTGCCATCCTCCTGCCGGTGA
CTCTGACCTTCCAGATCTAGGGGGGCCTGGGGAGCCCCCAATCCAGCCTG
GGCACGTCCCCTCCCCTAGGCCACAGCCGAGGTCACAATCAACATTCATT
GTTGTCGGTGGGTTGTGAGGACTGAGGCCAGACCCACCGGGGGATGAATG
TCACTGTGGCTGGGCCAGACACGGCTTAAGGGGAATGGGGACTGGGGACA
GGACCCCCCACCGCCACAGTCACTCAGCCTGTTTTTTGCCCTGACCCCAA
CCACTCCTCTTTGGAGAGGAGAGCTGGTGTCTGGAATC.

As used herein, miR-148a-5p refers to a human miR-148a (hsa-miR-148a -5p; miRBase Accession No: MIMAT0004549). An example of a mature human miR-148a -5p includes a nucleic acid sequence that is at least 70% (e.g., at least 75%, at least 80%, at least 85%, at least 90%, at least 95%, at least 99%, or at least 100%) identical to:

| (SEQ ID NO: 50) | |
|---|---|
| | AAAGUUCUGAGACACUCCGACU. |

An example of a stem-loop precursor miRNA (pre-miRNA) that can be used to generate mature human miR-148a includes a nucleic acid sequence that is at least 70% (e.g., at least 75%, at least 80%, at least 85%, at least 90%, at least 95%, at least 99%, or at least 100%) identical to:

(miRBase Accession No: MI0000253)
(SEQ ID NO: 51)
GAGGCAAAGUUCUGAGACACUCCGACUCUGAGUAUGAUAGAAGUCAGUGC
ACUACAGAACUUUGUCUC.

As used herein, miR-107-5p refers to a human miR-107 (hsa-miR-107-5p; miRBase Accession No: MIMAT0000104). An example of a mature human miR-107-5p includes a nucleic acid sequence that is at least 70% (e.g., at least 75%, at least 80%, at least 85%, at least 90%, at least 95%, at least 99%, or at least 100%) identical to:

| (SEQ ID NO: 52) | |
|---|---|
| | AGCAGCAUUGUACAGGGCUAUCA. |

An example of a stem-loop precursor miRNA (pre-miRNA) that can be used to generate mature human miR-107 includes a nucleic acid sequence that is at least 70% (e.g., at least 75%, at least 80%, at least 85%, at least 90%, at least 95%, at least 99%, or at least 100%) identical to:

(miRBase Accession No: MI0000114)
(SEQ ID NO: 53)
CUCUCUGCUUUCAGCUUCUUUACAGUGUUGCCUUGUGGCAUGGAGUUCAA
GCAGCAUUGUACAGGGCUAUCAAAGCACAGA.

### Methods of Producing T Cells

As described herein, any appropriate method of producing cells (e.g., T cells) comprising a FOXP3 polypeptide and a microRNA (e.g., one or more of any of the microRNAs described herein) can be used to generate the T cells as described herein. In some embodiments, a cell (e.g., a T cell) that is transduced with the nucleic acid sequences described herein is isolated from a mammal (e.g., a human) using any appropriate method (e.g., magnetic activated sorting or flow cytometry-mediated sorting). For example, a T cell can be made by transducing nucleic acid sequences encoding a FOXP3 polypeptide and a microRNA into a cell (e.g., any of the T cells described herein) using a lentivirus. In all cases described herein, the nucleic acid sequences are operably linked to a promoter or are operably linked to other nucleic acid sequences using a self-cleaving 2A polypeptide or IRES sequence.

Methods of introducing nucleic acids and expression vectors into a cell (e.g., an eukaryotic cell) are known in the art. Non-limiting examples of methods that can be used to introduce a nucleic acid into a cell include lipofection, transfection, electroporation, microinjection, calcium phosphate transfection, dendrimer-based transfection, cationic polymer transfection, cell squeezing, sonoporation, optical transfection, impalefection, hydrodynamic delivery, magnetofection, viral transduction (e.g., adenoviral and lentiviral transduction), and nanoparticle transfection. As used herein, “transformed” and “transduced” are used interchangeably.

### Nucleic Acids/Vectors

Also provided herein are nucleic acids sequences that encode any of the FOXP3 polypeptides and/or any of the microRNAs described herein. For example, nucleic acid sequences are included that encode for a FOXP3 polypeptide (e.g., any of the FOXP3 polypeptides described herein) and a microRNA (e.g., one or more of any of the microRNAs described herein).

Also provided herein are vectors that include any of the nucleic acids encoding any of the FOXP3 polypeptides and/or any of the microRNAs described herein.

Also provided herein is a set of vectors that include two or more vectors. For example, the set of vectors includes a first vector that includes a nucleic acid encoding any of the FOXP3 polypeptides described herein and a second vector that includes a nucleic acid encoding a miRNA (e.g., one or more of any of the exemplary miRNAs described herein).

In some embodiments, the set of vectors is combined prior to transduction (e.g., combined prior to transfection into a mammalian cell where the cell is used to produce the polypeptide and/or miRNA or the cell is used to produce a virus that includes the nucleic acids contained in the set of vectors).

Any of the vectors described herein can be an expression vector. For example, an expression vector can include a promoter sequence operably linked to a first sequence encoding any of the FOXP3 polypeptides as described herein or a second sequence encoding any of the miRNAs described herein. Non-limiting examples of vectors include plasmids, transposons, cosmids, and viral vectors (e.g., any adenoviral vectors (e.g., pSV or pCMV vectors), adeno-associated virus (AAV) vectors, lentivirus vectors, and retroviral vectors), and any Gateway® vectors. In some cases, a vector can include sufficient cis-acting elements that supplement expression where the remaining elements needed for expression can be supplied by the host mammalian cell or in an in vitro expression system. Skilled practitioners will be capable of selecting suitable vectors for making any of the T cells as described herein. Any appropriate promoter (e.g., EF1 alpha) can be operably linked to any of the nucleic acid sequences described herein. Non-limiting examples of promoters to be used in any of the vectors or constructs described herein include EF1a, SFFV, PGK, CMV, CAG, UbC, MSCV, MND, EF1a hybrid, and/or CAG hybrid. As used herein, the term “operably linked” is well known in the art and refers to genetic components that are combined such that they carry out their normal functions. For example, a nucleic acid sequence is operably linked to a promoter when its transcription is under the control of the promoter. In another example, a nucleic acid sequence can be operably linked to other nucleic acid sequence by a self-cleaving 2A polypeptide or an internal ribosome entry site (IRES). In such cases, the self-cleaving 2A polypeptide allows the second nucleic acid to be under the control of the promoter operably linked to the first nucleic acid sequence. The nucleic acid sequences described herein can be operably linked to a promoter. In some cases, the nucleic acid sequences described herein can be operably linked to any other nucleic acid sequence described herein using a self-cleaving 2A polypeptide or IRES. In some cases, the nucleic acid sequences are all included on one vector and operably linked either to a promoter upstream of the nucleic acid sequences or operably linked to the other nucleic acid sequences through a self-cleaving 2A polypeptide or an IRES.

### Compositions

Also provided herein are compositions (e.g., pharmaceutical compositions) that include any of the cells described herein, a population of any of the cells described herein, or any of the nucleic acids or vectors described herein. In some embodiments, the compositions include any of the T cells (e.g., any of the T cells described herein, including any of the T cells produced using any of the methods described herein). In some embodiments, the pharmaceutical compositions are formulated for different routes of administration (e.g., intravenous, subcutaneous). In some embodiments, the pharmaceutical compositions can include a pharmaceutically acceptable carrier (e.g., phosphate buffered saline).

### Cells

Also provided herein are T cells (e.g., any of the exemplary T cells described herein or known in the art) comprising any of the nucleic acids described herein that encode any of the FOXP3 polypeptides and/or any of the miRNAs described herein. Also provided herein are T cells (e.g., any of the exemplary T cells described herein or known in the art) that include any of the vectors described herein. In some embodiments, the T cells are any of the exemplary types of T cells described herein or known in the art.

The T cells can include, for example, mammalian (e.g., rodent, non-human primate, or human) T cells. Non-limiting examples of mammalian T cells include human T cells (e.g., autologous or allogeneic T cells).

In some embodiments, the T cells (e.g., any of the T cells described herein or known in the art) comprising any of the nucleic acids described herein that encode any of the FOXP3 polypeptides and any of the miRNAs described herein demonstrate increased (e.g., at least a 1% increase, at least a 5% increase, at least a 10% increase, at least a 15% increase, at least a 20% increase, at least a 25% increase, at least a 30% increase, at least a 35% increase, at least a 40% increase, at least a 45% increase, at least a 50% increase, at least a 55% increase, at least a 60% increase, at least a 65% increase, at least a 70% increase, at least a 75% increase, at least a 80% increase, at least a 85% increase, at least a 90% increase, at least a 95% increase, at least a 100% increase, or about a 1% increase to about a 100% increase, about a 1% increase to about a 95% increase, about a 1% increase to about a 90% increase, about a 1% increase to about a 85% increase, about a 1% increase to about a 80% increase, about a 1% increase to about a 75% increase, about a 1% increase to about a 70% increase, about a 1% increase to about a 65% increase, about a 1% increase to about a 60% increase, about a 1% increase to about a 55% increase, about a 1% increase to about a 50% increase, about a 1% increase to about a 45% increase, about a 1% increase to about a 40% increase, about a 1% increase to about a 35% increase, about a 1% increase to about a 30% increase, about a 1% increase to about a 25% increase, about a 1% increase to about a 20% increase, about a 1% increase to about a 15% increase, about a 1% increase to about a 10% increase, about a 1% increase to about a 5% increase, about a 5% increase to about a 100% increase, about a 5% increase to about a 95% increase, about a 5% increase to about a 90% increase, about a 5% increase to about a 85% increase, about a 5% increase to about a 80% increase, about a 5% increase to about a 75% increase, about a 5% increase to about a 70% increase, about a 5% increase to about a 65% increase, about a 5% increase to about a 60% increase, about a 5% increase to about a 55% increase, about a 5% increase to about a 50% increase, about a 5% increase to about a 45% increase, about a 5% increase to about a 40% increase, about a 5% increase to about a 35% increase, about a 5% increase to about a 30% increase, about a 5% increase to about a 25% increase, about a 5% increase to about a 20% increase, about a 5% increase to about a 15% increase, about a 5% increase to about a 10% increase, about a 10% increase to about a 100% increase, about a 10% increase to about a 95% increase, about a 10% increase to about a 90% increase, about a 10% increase to about a 85% increase, about a 10% increase to about a 80% increase, about a 10% increase to about a 75% increase, about a 10% increase to about a 70% increase, about a 10% increase to about a 65% increase, about a 10% increase to about a 60% increase, about a 10% increase to about a 55% increase, about a 10% increase to about a 50% increase, about a 10% increase to about a 45% increase, about a 10% increase to about a 40% increase, about a 10% increase to about a 35% increase, about a 10% increase to about a 30% increase, about a 10% increase to about a 25% increase, about a 10% increase to about a 20% increase, about a 10% increase to about a 15% increase, about a 15% increase to about a 100% increase, about a 15% increase to about a 95% increase, about a 15% increase to about a 90% increase, about a 15% increase to about a 85% increase, about a 15% increase to about a 80% increase, about a 15% increase to about a 75% increase, about a 15% increase to about a 70% increase, about a 15% increase to about a 65% increase, about a 15% increase to about a 60% increase, about a 15% increase to about a 55% increase, about a 15% increase to about a 50% increase, about a 15% increase to about a 45% increase, about a 15% increase to about a 40% increase, about a 15% increase to about a 35% increase, about a 15% increase to about a 30% increase, about a 15% increase to about a 25% increase, about a 15% increase to about a 20% increase, about a 20% increase to about a 100% increase, about a 20% increase to about a 95% increase, about a 20% increase to about a 90% increase, about a 20% increase to about a 85% increase, about a 20% increase to about a 80% increase, about a 20% increase to about a 75% increase, about a 20% increase to about a 70% increase, about a 20% increase to about a 65% increase, about a 20% increase to about a 60% increase, about a 20% increase to about a 55% increase, about a 20% increase to about a 50% increase, about a 20% increase to about a 45% increase, about a 20% increase to about a 40% increase, about a 20% increase to about a 35% increase, about a 20% increase to about a 30% increase, about a 20% increase to about a 25% increase, about a 25% increase to about a 100% increase, about a 25% increase to about a 95% increase, about a 25% increase to about a 90% increase, about a 25% increase to about a 85% increase, about a 25% increase to about a 80% increase, about a 25% increase to about a 75% increase, about a 25% increase to about a 70% increase, about a 25% increase to about a 65% increase, about a 25% increase to about a 60% increase, about a 25% increase to about a 55% increase, about a 25% increase to about a 50% increase, about a 25% increase to about a 45% increase, about a 25% increase to about a 40% increase, about a 25% increase to about a 35% increase, about a 25% increase to about a 30% increase, about a 30% increase to about a 100% increase, about a 30% increase to about a 95% increase, about a 30% increase to about a 90% increase, about a 30% increase to about a 85% increase, about a 30% increase to about a 80% increase, about a 30% increase to about a 75% increase, about a 30% increase to about a 70% increase, about a 30% increase to about a 65% increase, about a 30% increase to about a 60% increase, about a 30% increase to about a 55% increase, about a 30% increase to about a 50% increase, about a 30% increase to about a 45% increase, about a 30% increase to about a 40% increase, about a 30% increase to about a 35% increase, about a 35% increase to about a 100% increase, about a 35% increase to about a 95% increase, about a 35% increase to about a 90% increase, about a 35% increase to about a 85% increase, about a 35% increase to about a 80% increase, about a 35% increase to about a 75% increase, about a 35% increase to about a 70% increase, about a 35% increase to about a 65% increase, about a 35% increase to about a 60% increase, about a 35% increase to about a 55% increase, about a 35% increase to about a 50% increase, about a 35% increase to about a 45% increase, about a 35% increase to about a 40% increase, about a 40% increase to about a 100% increase, about a 40% increase to about a 95% increase, about a 40% increase to about a 90% increase, about a 40% increase to about a 85% increase, about a 40% increase to about a 80% increase, about a 40% increase to about a 75% increase, about a 40% increase to about a 70% increase, about a 40% increase to about a 65% increase, about a 40% increase to about a 60% increase, about a 40% increase to about a 55% increase, about a 40% increase to about a 50% increase, about a 40% increase to about a 45% increase, about a 45% increase to about a 100% increase, about a 45% increase to about a 95% increase, about a 45% increase to about a 90% increase, about a 45% increase to about a 85% increase, about a 45% increase to about a 80% increase, about a 45% increase to about a 75% increase, about a 45% increase to about a 70% increase, about a 45% increase to about a 65% increase, about a 45% increase to about a 60% increase, about a 45% increase to about a 55% increase, about a 45% increase to about a 50% increase, about a 50% increase to about a 100% increase, about a 50% increase to about a 95% increase, about a 50% increase to about a 90% increase, about a 50% increase to about a 85% increase, about a 50% increase to about a 80% increase, about a 50% increase to about a 75% increase, about a 50% increase to about a 70% increase, about a 50% increase to about a 65% increase, about a 50% increase to about a 60% increase, about a 50% increase to about a 55% increase, about a 55% increase to about a 100% increase, about a 55% increase to about a 95% increase, about a 55% increase to about a 90% increase, about a 55% increase to about a 85% increase, about a 55% increase to about a 80% increase, about a 55% increase to about a 75% increase, about a 55% increase to about a 70% increase, about a 55% increase to about a 65% increase, about a 55% increase to about a 60% increase, about a 60% increase to about a 100% increase, about a 60% increase to about a 95% increase, about a 60% increase to about a 90% increase, about a 60% increase to about a 85% increase, about a 60% increase to about a 80% increase, about a 60% increase to about a 75% increase, about a 60% increase to about a 70% increase, about a 60% increase to about a 65% increase, about a 65% increase to about a 100% increase, about a 65% increase to about a 95% increase, about a 65% increase to about a 90% increase, about a 65% increase to about a 85% increase, about a 65% increase to about a 80% increase, about a 65% increase to about a 75% increase, about a 65% increase to about a 70% increase, about a 70% increase to about a 100% increase, about a 70% increase to about a 95% increase, about a 70% increase to about a 90% increase, about a 70% increase to about a 85% increase, about a 70% increase to about a 80% increase, about a 70% increase to about a 75% increase, about a 75% increase to about a 100% increase, about a 75% increase to about a 95% increase, about a 75% increase to about a 90% increase, about a 75% increase to about a 85% increase, about a 75% increase to about a 80% increase, about a 80% increase to about a 100% increase, about a 80% increase to about a 95% increase, about a 80% increase to about a 90% increase, about a 80% increase to about a 85% increase, about a 85% increase to about a 100% increase, about a 85% increase to about a 95% increase, about a 85% increase to about a 90% increase, about a 90% increase to about a 100% increase, about a 90% increase to about a 95% increase, or about a 95% increase to about a 100% increase) growth (e.g., expansion), e.g., as compared to T cells comprising a nucleic acid encoding any of the FOXP3 polypeptides or any of the miRNAs described herein alone, when cultured under similar conditions. In some embodiments, T cells comprising any of the nucleic acids described herein that encode any of the FOXP3 polypeptides and any of the miRNAs described herein demonstrate increased (e.g., at least a 1% increase, at least a 5% increase, at least a 10% increase, at least a 15% increase, at least a 20% increase, at least a 25% increase, at least a 30% increase, at least a 35% increase, at least a 40% increase, at least a 45% increase, at least a 50% increase, at least a 55% increase, at least a 60% increase, at least a 65% increase, at least a 70% increase, at least a 75% increase, at least a 80% increase, at least a 85% increase, at least a 90% increase, at least a 95% increase, at least a 100% increase, or about a 1% increase to about a 100% increase (or any of the subranges of this range described herein) survival post-transduction, e.g., as compared to T cells comprising a nucleic acid encoding any of the FOXP3 polypeptides or any of the miRNAs described herein alone, when cultured under similar growth conditions. In some embodiments, the T cells (e.g., any of the T cells described herein or known in the art) comprising any of the nucleic acids described herein that encode any of the FOXP3 polypeptides and any of the miRNAs described herein demonstrate increased (e.g., at least a 1% increase, at least a 5% increase, at least a 10% increase, at least a 15% increase, at least a 20% increase, at least a 25% increase, at least a 30% increase, at least a 35% increase, at least a 40% increase, at least a 45% increase, at least a 50% increase, at least a 55% increase, at least a 60% increase, at least a 65% increase, at least a 70% increase, at least a 75% increase, at least a 80% increase, at least a 85% increase, at least a 90% increase, at least a 95% increase, at least a 100% increase, or about a 1% increase to about a 100% increase (or any of the subranges of this range described herein) growth (e.g., expansion) and increased (e.g., at least a 1% increase, at least a 5% increase, at least a 10% increase, at least a 15% increase, at least a 20% increase, at least a 25% increase, at least a 30% increase, at least a 35% increase, at least a 40% increase, at least a 45% increase, at least a 50% increase, at least a 55% increase, at least a 60% increase, at least a 65% increase, at least a 70% increase, at least a 75% increase, at least a 80% increase, at least a 85% increase, at least a 90% increase, at least a 95% increase, at least a 100% increase, or about a 1% increase to about a 100% increase (or any of the subranges of this range described herein) survival post-transduction, e.g., as compared to T cells comprising a nucleic acid encoding any of the FOXP3 polypeptides or any of the miRNAs described herein alone, when cultured under similar growth conditions.

In some embodiments, the T cells (e.g., any of the T cells described herein or known in the art) comprising a nucleic acid that encodes any of the FOXP3 polypeptides described herein and a nucleic acid encoding any of the miRNAs described herein result in a lack or decrease in T cells comprising a nucleic acid that encodes any of the FOXP3 polypeptides described herein or a nucleic acid encoding any of the miRNAs described herein alone, post-transduction. In some embodiments, the T cells (e.g., any of the T cells described herein or known in the art) comprising a nucleic acid that encodes any of the FOXP3 polypeptides described herein and a nucleic acid that encodes any of the miRNAs described herein enrich the number of T cells (e.g., any of the T cells described herein or known in the art) comprising a nucleic acid encoding any of the FOXP3 polypeptides described herein and a nucleic acid encoding any of the miRNAs described herein (e.g., miR10a, miR95, miR29a, miR150, and miR101) and decrease the number of T cells that lack a nucleic acid encoding any of the FOXP3 polypeptides described herein and lack a nucleic acid encoding any of the miRNAs described herein, post-transduction. In some embodiments, an increase in the number of T cells comprising a nucleic acid encoding any of the FOXP3 polypeptides described herein and a nucleic acid encoding any of the miRNAs described herein, post transduction, and/or the decrease in the number of T-cells not including a nucleic acid encoding any of the FOXP3 polypeptides described herein and not including a nucleic acid encoding any of the miRNAs described herein, post transduction, is assessed at day 2, day 3, day 4, day 5, day 6, day 7, day 8, day 9, day 10, day 11, day 12, day 13, day 14, day 15, day 16, day 17, day 18, day 19, day 20, day 21, day 22, day 23, day 24, day 25, day 26, day 27, day 28, day 29, or at day 30 post-transduction.

In some embodiments, the T cells (e.g., any of the T cells described herein or known in the art) comprising a nucleic acid that encodes any of the FOXP3 polypeptides described herein and a nucleic acid encoding miR-155 (e.g., any of the miR-155 nucleic acids described herein) result in increased (e.g., at least a 1% increase, at least a 5% increase, at least a 10% increase, at least a 15% increase, at least a 20% increase, at least a 25% increase, at least a 30% increase, at least a 35% increase, at least a 40% increase, at least a 45% increase, or at least a 50% increase, at least a 55% increase, at least a 60% increase, at least a 65% increase, at least a 70% increase, at least a 75% increase, at least a 80% increase, at least a 85% increase, at least a 85% increase, at least a 90% increase, at least a 95% increase, or at least a 100% increase, or about a 1% increase to about a 100% increase (or any of the subranges of this range described herein) survival post-transduction in the presence of IL-2, e.g., as compared to T cells not including a nucleic acid that encodes any of the FOXP3 polypeptides and/or not including a nucleic acid encoding miR-155 (e.g., any of the miR-155 nucleic acids described herein), in the presence of the same concentration of IL-2.

In some embodiments the concentration of IL-2 is about 0.01 mM to about 1000 mM, about 0.1 mM to about 900 mM, about 0.1 mM to about 800 mM, about 0.1 mM to about 700 mM, about 0.1 mM to about 600 mM, about 0.1 mM to about 500 mM, about 0.1 mM to about 400 mM, about 0.1 mM to about 300 mM, about 0.1 mM to about 200 mM, about 0.1 mM to about 100 mM, about 0.1 mM to about 90 mM, about 0.1 mM to about 80 mM, about 0.1 mM to about 70 mM, about 0.1 mM to about 60 mM, about 0.1 mM to about 50 mM, about 0.1 mM to about 40 mM, about 0.1 mM to about 30 mM, about 0.1 mM to about 20 mM, about 0.1 mM to about 10 mM, about 0.1 mM to about 1 mM, about 1.0 mM to about 900 mM, about 1.0 mM to about 800 mM, about 1.0 mM to about 700 mM, about 1.0 mM to about 600 mM, about 1.0 mM to about 500 mM, about 1.0 mM to about 400 mM, about 1.0 mM to about 300 mM, about 1.0 mM to about 200 mM, about 1.0 mM to about 100 mM, about 1.0 mM to about 90 mM, about 1.0 mM to about 80 mM, about 1.0 mM to about 70 mM, about 1.0 mM to about 60 mM, about 1.0 mM to about 50 mM, about 1.0 mM to about 40 mM, about 1.0 mM to about 30 mM, about 1.0 mM to about 20 mM, about 1.0 mM to about 10 mM, or about 1.0 mM to about 1 mM.

In some embodiments, the increased survival is relative to T cells transduced with FOXP3 alone in the presence of the same concentration of IL-2, e.g., as measured by FACS. For example, the activated T cells can be stained with a viability dye and Annexin V, which stains phosphatidylserine on the cell membrane and is indicative of apoptotic cells. In some embodiments, the increased survival of T cells transduced with a nucleic acid that encode any of the FOXP3 polypeptides described herein and miR-155 (e.g., any of the miR-155 nucleic acids described herein) relative to T cells transduced with a nucleic acid encoding FOXP3 alone in the presence of IL-2 is at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, or about a 1% increase to about a 100% increase (or any of the subranges of this range described herein)), e.g., as measured by FACS.

In some embodiments, the T cells (e.g., any of the T cells described herein or known in the art) comprising a nucleic acid that encodes any of the FOXP3 polypeptides described herein and miR-155 (e.g., any of the miR-155 nucleic acids described herein) results in a decrease (e.g., at least a 1% decrease, at least a 2% decrease, at least a 4% decrease, at least a 6% decrease, at least a 8% decrease, at least a 10% decrease, at least a 15% decrease, at least a 20% decrease, at least a 25% decrease, at least a 30% decrease, at least a 35% decrease, at least a 40% decrease at least a 45% decrease, at least a 50% decrease, at least a 55% decrease, at least a 60% decrease, at least a 65% decrease, at least a 70% decrease, at least a 75% decrease, at least a 80% decrease, at least a 85% decrease, at least a 90% decrease, at least a 95% decrease, or at least a 99% decrease, or about a 1% decrease to about a 99% decrease, about a 1% decrease to about a 95% decrease, about a 1% decrease to about a 90% decrease, about a 1% decrease to about a 85% decrease, about a 1% decrease to about a 80% decrease, about a 1% decrease to about a 75% decrease, about a 1% decrease to about a 70% decrease, about a 1% decrease to about a 65% decrease, about a 1% decrease to about a 60% decrease, about a 1% decrease to about a 55% decrease, about a 1% decrease to about a 50% decrease, about a 1% decrease to about a 45% decrease, about a 1% decrease to about a 40% decrease, about a 1% decrease to about a 35% decrease, about a 1% decrease to about a 30% decrease, about a 1% decrease to about a 25% decrease, about a 1% decrease to about a 20% decrease, about a 1% decrease to about a 15% decrease, about a 1% decrease to about a 10% decrease, about a 1% decrease to about a 5% decrease, about a 5% decrease to about a 99% decrease, about a 5% decrease to about a 95% decrease, about a 5% decrease to about a 90% decrease, about a 5% decrease to about a 85% decrease, about a 5% decrease to about a 80% decrease, about a 5% decrease to about a 75% decrease, about a 5% decrease to about a 70% decrease, about a 5% decrease to about a 65% decrease, about a 5% decrease to about a 60% decrease, about a 5% decrease to about a 55% decrease, about a 5% decrease to about a 50% decrease, about a 5% decrease to about a 45% decrease, about a 5% decrease to about a 40% decrease, about a 5% decrease to about a 35% decrease, about a 5% decrease to about a 30% decrease, about a 5% decrease to about a 25% decrease, about a 5% decrease to about a 20% decrease, about a 5% decrease to about a 15% decrease, about a 5% decrease to about a 10% decrease, about a 10% decrease to about a 99% decrease, about a 10% decrease to about a 95% decrease, about a 10% decrease to about a 90% decrease, about a 10% decrease to about a 85% decrease, about a 10% decrease to about a 80% decrease, about a 10% decrease to about a 75% decrease, about a 10% decrease to about a 70% decrease, about a 10% decrease to about a 65% decrease, about a 10% decrease to about a 60% decrease, about a 10% decrease to about a 55% decrease, about a 10% decrease to about a 50% decrease, about a 10% decrease to about a 45% decrease, about a 10% decrease to about a 40% decrease, about a 10% decrease to about a 35% decrease, about a 10% decrease to about a 30% decrease, about a 10% decrease to about a 25% decrease, about a 10% decrease to about a 20% decrease, about a 10% decrease to about a 15% decrease, about a 15% decrease to about a 99% decrease, about a 15% decrease to about a 95% decrease, about a 15% decrease to about a 90% decrease, about a 15% decrease to about a 85% decrease, about a 15% decrease to about a 80% decrease, about a 15% decrease to about a 75% decrease, about a 15% decrease to about a 70% decrease, about a 15% decrease to about a 65% decrease, about a 15% decrease to about a 60% decrease, about a 15% decrease to about a 55% decrease, about a 15% decrease to about a 50% decrease, about a 15% decrease to about a 45% decrease, about a 15% decrease to about a 40% decrease, about a 15% decrease to about a 35% decrease, about a 15% decrease to about a 30% decrease, about a 15% decrease to about a 25% decrease, about a 15% decrease to about a 20% decrease, about a 20% decrease to about a 99% decrease, about a 20% decrease to about a 95% decrease, about a 20% decrease to about a 90% decrease, about a 20% decrease to about a 85% decrease, about a 20% decrease to about a 80% decrease, about a 20% decrease to about a 75% decrease, about a 20% decrease to about a 70% decrease, about a 20% decrease to about a 65% decrease, about a 20% decrease to about a 60% decrease, about a 20% decrease to about a 55% decrease, about a 20% decrease to about a 50% decrease, about a 20% decrease to about a 45% decrease, about a 20% decrease to about a 40% decrease, about a 20% decrease to about a 35% decrease, about a 20% decrease to about a 30% decrease, about a 20% decrease to about a 25% decrease, about a 25% decrease to about a 99% decrease, about a 25% decrease to about a 95% decrease, about a 25% decrease to about a 90% decrease, about a 25% decrease to about a 85% decrease, about a 25% decrease to about a 80% decrease, about a 25% decrease to about a 75% decrease, about a 25% decrease to about a 70% decrease, about a 25% decrease to about a 65% decrease, about a 25% decrease to about a 60% decrease, about a 25% decrease to about a 55% decrease, about a 25% decrease to about a 50% decrease, about a 25% decrease to about a 45% decrease, about a 25% decrease to about a 40% decrease, about a 25% decrease to about a 35% decrease, about a 25% decrease to about a 30% decrease, about a 30% decrease to about a 99% decrease, about a 30% decrease to about a 95% decrease, about a 30% decrease to about a 90% decrease, about a 30% decrease to about a 85% decrease, about a 30% decrease to about a 80% decrease, about a 30% decrease to about a 75% decrease, about a 30% decrease to about a 70% decrease, about a 30% decrease to about a 65% decrease, about a 30% decrease to about a 60% decrease, about a 30% decrease to about a 55% decrease, about a 30% decrease to about a 50% decrease, about a 30% decrease to about a 45% decrease, about a 30% decrease to about a 40% decrease, about a 30% decrease to about a 35% decrease, about a 35% decrease to about a 99% decrease, about a 35% decrease to about a 95% decrease, about a 35% decrease to about a 90% decrease, about a 35% decrease to about a 85% decrease, about a 35% decrease to about a 80% decrease, about a 35% decrease to about a 75% decrease, about a 35% decrease to about a 70% decrease, about a 35% decrease to about a 65% decrease, about a 35% decrease to about a 60% decrease, about a 35% decrease to about a 55% decrease, about a 35% decrease to about a 50% decrease, about a 35% decrease to about a 45% decrease, about a 35% decrease to about a 40% decrease, about a 40% decrease to about a 99% decrease, about a 40% decrease to about a 95% decrease, about a 40% decrease to about a 90% decrease, about a 40% decrease to about a 85% decrease, about a 40% decrease to about a 80% decrease, about a 40% decrease to about a 75% decrease, about a 40% decrease to about a 70% decrease, about a 40% decrease to about a 65% decrease, about a 40% decrease to about a 60% decrease, about a 40% decrease to about a 55% decrease, about a 40% decrease to about a 50% decrease, about a 40% decrease to about a 45% decrease, about a 45% decrease to about a 99% decrease, about a 45% decrease to about a 95% decrease, about a 45% decrease to about a 90% decrease, about a 45% decrease to about a 85% decrease, about a 45% decrease to about a 80% decrease, about a 45% decrease to about a 75% decrease, about a 45% decrease to about a 70% decrease, about a 45% decrease to about a 65% decrease, about a 45% decrease to about a 60% decrease, about a 45% decrease to about a 55% decrease, about a 45% decrease to about a 50% decrease, about a 50% decrease to about a 99% decrease, about a 50% decrease to about a 95% decrease, about a 50% decrease to about a 90% decrease, about a 50% decrease to about a 85% decrease, about a 50% decrease to about a 80% decrease, about a 50% decrease to about a 75% decrease, about a 50% decrease to about a 70% decrease, about a 50% decrease to about a 65% decrease, about a 50% decrease to about a 60% decrease, about a 50% decrease to about a 55% decrease, about a 55% decrease to about a 99% decrease, about a 55% decrease to about a 95% decrease, about a 55% decrease to about a 90% decrease, about a 55% decrease to about a 85% decrease, about a 55% decrease to about a 80% decrease, about a 55% decrease to about a 75% decrease, about a 55% decrease to about a 70% decrease, about a 55% decrease to about a 65% decrease, about a 55% decrease to about a 60% decrease, about a 60% decrease to about a 99% decrease, about a 60% decrease to about a 95% decrease, about a 60% decrease to about a 90% decrease, about a 60% decrease to about a 85% decrease, about a 60% decrease to about a 80% decrease, about a 60% decrease to about a 75% decrease, about a 60% decrease to about a 70% decrease, about a 60% decrease to about a 65% decrease, about a 65% decrease to about a 99% decrease, about a 65% decrease to about a 95% decrease, about a 65% decrease to about a 90% decrease, about a 65% decrease to about a 85% decrease, about a 65% decrease to about a 80% decrease, about a 65% decrease to about a 75% decrease, about a 65% decrease to about a 70% decrease, about a 70% decrease to about a 99% decrease, about a 70% decrease to about a 95% decrease, about a 70% decrease to about a 90% decrease, about a 70% decrease to about a 85% decrease, about a 70% decrease to about a 80% decrease, about a 70% decrease to about a 75% decrease, about a 75% decrease to about a 99% decrease, about a 75% decrease to about a 95% decrease, about a 75% decrease to about a 90% decrease, about a 75% decrease to about a 85% decrease, about a 75% decrease to about a 80% decrease, about a 80% decrease to about a 99% decrease, about a 80% decrease to about a 95% decrease, about a 80% decrease to about a 90% decrease, about a 80% decrease to about a 85% decrease, about a 85% decrease to about a 99% decrease, about a 85% decrease to about a 95% decrease, about a 85% decrease to about a 90% decrease, about a 90% decrease to about a 99% decrease, about a 90% decrease to about a 95% decrease, or about a 95% decrease to about a 99% decrease) in the percentage of necrotic T cells post-transduction (e.g., one, two, three, four, five, six, seven, eight, nine, ten, eleven, or twelve days post-transduction), e.g., as compared to T cells comprising a nucleic acid that encodes any of the FOXP3 polypeptides described herein or a nucleic acid that encodes miR155 alone. In some embodiments, the decrease in the percentage of necrotic T cells post-transduction is relative to T cells transduced with a nucleic acid encoding any of the FOXP3 polypeptides described herein alone in the presence of the same concentration of IL-2 is at least a 1% decrease, at least a 2% decrease, at least a 4% decrease, at least a 6% decrease, at least a 8% decrease, at least a 10% decrease, at least a 15% decrease, at least a 20% decrease, at least a 25% decrease, at least a 30% decrease, at least a 35% decrease, at least a 40% decrease at least a 45% decrease, at least a 50% decrease, at least a 55% decrease, at least a 60% decrease, at least a 65% decrease, at least a 70% decrease, at least a 75% decrease, at least a 80% decrease, at least a 85% decrease, at least a 90% decrease, at least a 95% decrease, or at least a 99% decrease, or about a 1% decrease to about a 99% decrease (or any of the subranges of this range described herein), e.g., as measured by FACS.

In some embodiments, the T cells (e.g., any of the T cells described herein or known in the art) comprising a nucleic acid that encodes any of the FOXP3 polypeptides described herein and miR-155 (e.g., any of the miR-155 nucleic acids described herein) result in a decrease (e.g., at least a 1% decrease, at least a 2% decrease, at least a 4% decrease, at least a 6% decrease, at least a 8% decrease, at least a 10% decrease, at least a 15% decrease, at least a 20% decrease, at least a 25% decrease, at least a 30% decrease, at least a 35% decrease, at least a 40% decrease at least a 45% decrease, at least a 50% decrease, at least a 55% decrease, at least a 60% decrease, at least a 65% decrease, at least a 70% decrease, at least a 75% decrease, at least a 80% decrease, at least a 85% decrease, at least a 90% decrease, at least a 95% decrease, or at least a 99% decrease, or about a 1% decrease to about a 99% decrease (or any of the subranges of this range described herein)) in the number of early apoptotic T cells post transduction, e.g., as compared to T cells comprising a nucleic acid that encodes any of the FOXP3 polypeptides described herein or a nucleic acid that encodes miR155 alone when cultured using the same conditions. In some embodiments, the decrease in the number of early apoptotic T cells post-transduction is relative to T cells transduced with a nucleic acid encoding a FOXP3 polypeptide alone in the presence of IL-2 is at least a 1% decrease, at least a 2% decrease, at least a 4% decrease, at least a 6% decrease, at least a 8% decrease, at least a 10% decrease, at least a 15% decrease, at least a 20% decrease, at least a 25% decrease, at least a 30% decrease, at least a 35% decrease, at least a 40% decrease at least a 45% decrease, at least a 50% decrease, at least a 55% decrease, at least a 60% decrease, at least a 65% decrease, at least a 70% decrease, at least a 75% decrease, at least a 80% decrease, at least a 85% decrease, at least a 90% decrease, at least a 95% decrease, or at least a 99% decrease, or about a 1% decrease to about a 99% decrease (or any of the subranges of this range described herein), e.g., as measured by FACS.

In some embodiments, the T cells (e.g., any of the T cells described herein or known in the art) comprising a nucleic acid that encodes any of the FOXP3 polypeptides described herein and miR-155 (e.g., any of the miR-155 nucleic acids described herein) result in a decrease (e.g., at least a 1% decrease, at least a 2% decrease, at least a 4% decrease, at least a 6% decrease, at least a 8% decrease, at least a 10% decrease, at least a 15% decrease, at least a 20% decrease, at least a 25% decrease, at least a 30% decrease, at least a 35% decrease, at least a 40% decrease at least a 45% decrease, at least a 50% decrease, at least a 55% decrease, at least a 60% decrease, at least a 65% decrease, at least a 70% decrease, at least a 75% decrease, at least a 80% decrease, at least a 85% decrease, at least a 90% decrease, at least a 95% decrease, or at least a 99% decrease, or about a 1% decrease to about a 99% decrease (or any of the subranges of this range described herein)) in the percentage of late apoptotic T cells post transduction, e.g., as compared to T cells comprising a nucleic acid that encodes any of the FOXP3 polypeptides described herein or a nucleic acid that encodes miR155 alone when cultured using the same conditions. In some embodiments, the decrease in the percentage of late apoptotic T cells post-transduction is relative to T cells transduced with a nucleic acid encoding a FOXP3 polypeptide alone in the presence of IL-2 is at least a 1% decrease, at least a 2% decrease, at least a 4% decrease, at least a 6% decrease, at least a 8% decrease, at least a 10% decrease, at least a 15% decrease, at least a 20% decrease, at least a 25% decrease, at least a 30% decrease, at least a 35% decrease, at least a 40% decrease at least a 45% decrease, at least a 50% decrease, at least a 55% decrease, at least a 60% decrease, at least a 65% decrease, at least a 70% decrease, at least a 75% decrease, at least a 80% decrease, at least a 85% decrease, at least a 90% decrease, at least a 95% decrease, or at least a 99% decrease, or about a 1% decrease to about a 99% decrease (or any of the subranges of this range described herein), e.g., as measured by FACS.

### Methods of Treatment

Also provided herein are methods of treating a mammal (e.g., a human) having an autoimmune disease that includes administering to the mammal (e.g., human) a therapeutically effective amount of a T cell (e.g., any of the exemplary T cells described herein) or any of the compositions (e.g., pharmaceutical compositions) described herein.

In some embodiments, these methods can result in a reduction in the number, severity, or frequency of one or more symptoms of the autoimmune diseases in the mammal (e.g., as compared to the number, severity, or frequency of the one or more symptoms of the autoimmune disease in the mammal prior to treatment). For example, a mammal having an autoimmune disease having been administered a T cell as described here can experience a reduction in inflammation or autoantibody production.

Any appropriate method of administration can be used to administer the T cells to a mammal (e.g. a human) having an autoimmune disease. Examples of methods of administration include, without limitation, parenteral administration and intravenous injection.

A pharmaceutical composition containing the T cells and a pharmaceutically acceptable carrier or buffer can be administered to a mammal (e.g., a human) having an autoimmune disease. For example, a pharmaceutical composition (e.g., a T cell along with a pharmaceutically acceptable carrier) to be administered to a mammal having an autoimmune disease can be formulated in an injectable form (e.g., emulsion, solution and/or suspension). In some embodiments, a pharmaceutical composition containing the T cells can include phosphate buffered saline.

Pharmaceutically acceptable carriers, fillers, and vehicles that can be used in a pharmaceutical composition described herein can include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Effective dosage can vary depending on the severity of the autoimmune disease, the route of administration, the age and general health condition of the subject, excipient usage, the possibility of co-usage with other therapeutic treatments, and the judgment of the treating physician. An effective amount of a T cell can be any amount that reduces inflammation and autoantibody production within a mammal having an autoimmune disease without producing significant toxicity to the mammal. For example, an effective amount of T cells administered to a mammal having an autoimmune disease can be from about 1×10⁶ cells to about 1×10¹⁰ (e.g., from about 1×10⁶ to about 1×10⁹, from about 1×10⁶ to about 1×10⁸, from about 1×10⁶ to about 1×10⁷, from about 1×10⁷ to about 1×10¹⁰, from about 1×10⁷ to about 1×10⁹, from about 1×10⁷ to about 1×10⁸, from about 1×10⁸ to about 1×10¹⁰, from about 1×10⁸ to about 1×10⁹, or form about 1×10⁹ to about 1×10¹⁰) cells. In some cases, the T cells can be a purified population of immune cells generated as described herein. In some cases, the purity of the population of T cells can be assessed using any appropriate method, including, without limitation, flow cytometry. In some cases, the population of T cells to be administered can include a range of purities from about 70% to about 100%, from about 70% to about 90%, from about 70% to about 80%, from about 80% to about 90%, from about 90% to about 100%, from about 80% to about 100%, from about 80% to about 90%, or from about 90% to 100%. In some cases, the dosage (e.g., number of T cells to be administered) can adjusted based on the level of purity of the T cells.

The frequency of administration of a T cell can be any frequency that reduces inflammation or autoantibody production within a mammal having an autoimmune disease without producing toxicity to the mammal. In some cases, the actual frequency of administration can vary depending on various factors including, without limitation, the effective amount, duration of treatment, use of multiple treatment agents, route of administration, and severity of the condition may require an increase or decrease in frequency of administration.

An effective duration for administering a composition containing a T cell can be any duration that reduces inflammation or autoantibody production within a mammal having an autoimmune disease without producing toxicity to the mammal. In some cases, the effective duration can vary from several days to several months. In general, the effective treatment duration for administering a composition containing a T cell to treat an autoimmune disease can range in duration from about one month to about five years (e.g., from about two months to about five years, from about three months to about five years, from about six months to about five years, from about eight months to about five years, from about one year to about five years, from about one month to about four years, from about one month to about three years, from about one month to about two years, from about six months to about four years, from about six months to about three years, or from about six months to about two years). In some cases, the effective treatment duration for administering a composition containing a T cell can be for the remainder of the life of the mammal.

In some cases, a course of treatment and/or the severity of one or more symptoms related to autoimmune disease can be monitored. Any appropriate method can be used to determine whether the autoimmune disease is being treated. For example, immunological techniques (e.g., ELISA) can be performed to determine if the level of autoantibodies present within a mammal being treated as described herein is reduced following the administration of the T cells. Remission and relapse of the disease can be monitored by testing for one or more markers of autoimmune disease.

Any appropriate autoimmune disease can be treated with a T cell as described herein. In some cases, an autoimmune disease caused by the accumulation of autoantibodies can be treated with a T cell as described herein. Examples of autoimmune diseases include, without limitation, lupus, rheumatoid arthritis, multiple sclerosis, insulin dependent diabetes mellitis, myasthenia gravis, Grave's disease, autoimmune hemolytic anemia, autoimmune thrombocytopenia purpura, Goodpasture's syndrome, pemphigus vulgaris, acute rheumatic fever, post-streptococcal glomerulonephritis, Crohn's disease, Celiac disease, and polyarteritis nodosa.

The invention will be further described in the following examples, which do not limit the scope of the invention described in the claims.

### EXAMPLES

### Example 1. T Cells Transduced with FOXP3 and miRNAs Show an Enhanced Treg Phenotype

A set of experiments was performed to assess the effect of co-expression of a miRNA and a FOXP3 polypeptide in T cells. In these experiments, CD4⁺ T cells were co-transduced with a lentivirus that included a nucleic acid sequence encoding a FOXP3 polypeptide and a second lentivirus that included a nucleic acid encoding a miRNA.

### Materials and Methods

### Cloning Lentiviral Constructs

Primary miRNA (mIR) sequences (for miR-142, miR-155, miR-15b, miR-16, miR-146a, miR-21a, miR-99a, miR-150, miR-10a, miR-95, miR-126, miR-29a, miR-24, miR-181c, and miR-101) were identified from miRBase.org and ordered as gBlocks from IDT with homologous overhangs for Gibson cloning into the pAldevron lentiviral backbone. All miRs were cloned 3′ of GFP under control of a constitutive SFFV promoter (FIG. 2B). After Gibson cloning, constructs were sequenced and DNA was prepped for lentiviral transduction.

TABLE 1
Primary miRNA sequences.

| SEQ ID | | |
|---|---|---|
| NO: | miRNA | Sequence |
| SEQ ID | miR-142 | CCTAGTCTCTACCTGAGTGTCTCTGAAACTGGGGGGATGG |
| NO: 5 | | GGTGGAGCCTTTAGGGGGAAGGGAAGAGGGAACTGAAG |
| | | AGGAAGTGGGGGAGGGAGGTAGAGGAGGCAAGTCTGGC |
| | | GCCATGCTGAGTCACCGCCCACAAGGCCCAGGGCGGGCC |
| | | CTCGGGGGGCCCTGGCAGGGTTGGGGGGATCTTAGGAAG |
| | | CCACAAGGAGGGCTGGGGGGCTCTTGGAGCAGGAGTCAG |
| | | GAGGCCTGGGCAGCCTGAAGAGTACACGCCGACGGACA |
| | | GACAGACAGTGCAGTCACCCATAAAGTAGAAAGCACTAC |
| | | TAACAGCACTGGAGGGTGTAGTGTTTCCTACTTTATGGAT |
| | | GAGTGTACTGTGGGCTTCGGAGATCACGCCACTGCTGCC |
| | | GCCCGCTGCCCGCCACCATCTTCCTCGGCGCTCGGGGACC |
| | | TCGTGTGACAGGTGAGCACCTTACGGCCCCTCCCTACCCT |
| | | GCCCAGATGCCTGAAAGGCCTCCATGGCTTTCCTGCCCTT |
| | | CCTGGTTCCGGACAGCTGGGGAAAGGCCACAGCAGCTCC |
| | | TCTGCTGCCCTGCAGTCTTTGGGGGCGGGGAGGGCTGGA |
| | | CATGTGGAACCCTGATGCAGCCGCAGCGTCAAGGACGAG |
| | | GAAGGGGTGGGAAGGGATGGTACGTGGAG |
| SEQ ID | miR-155 | TATGCCTAAAGGTAACAATGTCATCTTTTAATTGCCAATT |
| NO: 8 | | TCTCTACCACTTTCAAAAAATTACTTCCAAGGATTTAATG |
| | | AGCTCCTTCCTTTCAACAGAAAATGGACTATTTTCCTTTC |
| | | AGATTTACTATATGCTGTCACTCCAGCTTTATAACCGCAT |
| | | GTGCATACACAAACATTTCTTTCTCTCTTGCAGGTGGCAC |
| | | AAACCAGGAAGGGGAAATCTGTGGTTTAAATTCTTTATG |
| | | CCTCATCCTCTGAGTGCTGAAGGCTTGCTGTAGGCTGTAT |
| | | GCTGTTAATGCTAATCGTGATAGGGGTTTTTGCCTCCAAC |
| | | TGACTCCTACATATTAGCATTAACAGTGTATGATGCCTGT |
| | | TACTAGCATTCACATGGAACAAATTGCTGCCGTGGGAGG |
| | | ATGACAAAGAAGCATGAGTCACCCTGCTGGATAAACTTA |
| | | GACTTCAGGCTTTATCATTTTTCAATCTGTTAATCATAATC |
| | | TGGTCACTGGGATGTTCAACCTTAAACTAAGTTTTGAAAG |
| | | TAAGGTTATTTAAAAGATTTATCAGTAGTATCCTAAATGC |
| | | AAACATTTTCATTTAAATGTCAAGCCCATGTTTGTTTTTAT |
| | | CATTAACAGAAAATATATTCATGTCATTCTTAATTGCAGG |
| | | TTTTG |
| SEQ ID | miR-15b | TTTTCCTCAAAACAGGAAGGGGATGATTATGAAGTCATT |
| NO: 11 | | CCTAACAGTAATTTCTATGTATCCAGAACGGCCTGCAGA |
| | | GATAATACTTCTGTCTATCACATAAGTGGAAAGAAAAAG |
| | | ACATTTAAGGATGTTGGAAATCTTCTTCGAAGCCATGGA |
| | | ATTGACTTGGACCATAATAGATTTTTAATTTTACAGGTAA |
| | | GTTTATTAAAGACTTCAAAGATTCTCTTATTCTTGTTACTT |
| | | TTTTTTCTATAAAGCTAGGTTGGATGAATCCTACATTTTT |
| | | GAGGCCTTAAAGTACTGTAGCAGCACATCATGGTTTACA |
| | | TGCTACAGTCAAGATGCGAATCATTATTTGCTGCTCTAGA |
| | | AATTTAAGGAAATTCATTCAAAACTATGTTTTCATCATCA |
| | | GATGTTCGTTTTATGTTTGGATGAACTGACATACTTGTTC |
| | | CACTCTAGCAGCACGTAAATATTGGCGTAGTGAAATATA |
| | | TATTAAACACCAATATTACTGTGCTGCTTTAGTGTGACAG |
| | | GGATACAGCAACTATTTTATCAATTGTTTGTATTTCCCTTT |
| | | AAGGTAACATTTTAAATGAAATGTATTATATTTTAATCTA |
| | | TCCTTTTCCTTTGTTTTTGTTCTTATTATCTCTTCTGATATA |
| | | TAACCAAAAAATGAA |
| SEQ ID | miR-146a | TCAAGCGATCCTCCCACCACAGGCCATCATGCATGGCTC |
| NO: 14 | | ATTTTTTATTTTTAGTAGAGACAAATTCTCCATGTTGCCC |
| | | AGGCTAGTCCTGAACTCCTGGGCTCAAGAGATCCACCCA |
| | | CATCAGCCTTCCAGACTGCTGGCCTGGTCTCCTCCAGATG |
| | | TTTATAACTCATGAGTGCCAGGACTAGACCTGGTACTAG |
| | | GAAGCAGCTGCATTGGATTTACCAGGCTTTTCACTCTTGT |
| | | ATTTTACAGGGCTGGGACAGGCCTGGACTGCAAGGAGGG |
| | | GTCTTTGCACCATCTCTGAAAAGCCGATGTGTATCCTCAG |
| | | CTTTGAGAACTGAATTCCATGGGTTGTGTCAGTGTCAGAC |
| | | CTCTGAAATTCAGTTCTTCAGCTGGGATATCTCTGTCATC |
| | | GTGGGCTTGAGGACCTGGAGAGAGTAGATCCTGAAGAAC |
| | | TTTTTCAGTCTGCTGAAGAGCTTGGAAGACTGGAGACAG |
| | | AAGGCAGAGTCTCAGGCTCTGAAGGTATAAGGAGTGTGA |
| | | GTTCCTGTGAGAAACACTCATTTGATTGTGAAAAGACTTG |
| | | AATTCTATGCTAAGCAGGGTTCCAAGTAGCTAAATGAAT |
| | | GATCTCAGCAAGTCTCTCTTGCTGCTGCTGCTACTCGTTT |
| | | ACATTTATTGATTACTTACGATGATTCAGGTACTGTTGTA |
| | | AGTGCTTTACATG |
| SEQ ID | miR-21a | CAGTTTTCTTGCCGTTCTGTAAGTGTTTTATTCTTAGTGTG |
| NO: 19 | | ATTTTTTTCCATTGGGATGTTTTTGATTGAACTTGTTCATT |
| | | TTGTTTTGCTTGGGAGGAAAATAAACAATTTTACTTTTTT |
| | | CCTTTAGGAGCATTATGAGCATTATGTCAGAATAGAATA |
| | | GAATTGGGGTTCGATCTTAACAGGCCAGAAATGCCTGGG |
| | | TTTTTTTGGTTTGTTTTTGTTTTTGTTTTTTTATCAAATCCT |
| | | GCCTGACTGTCTGCTTGTTTTGCCTACCATCGTGACATCT |
| | | CCATGGCTGTACCACCTTGTCGGGTAGCTTATCAGACTGA |
| | | TGTTGACTGTTGAATCTCATGGCAACACCAGTCGATGGGC |
| | | TGTCTGACATTTTGGTATCTTTCATCTGACCATCCATATCC |
| | | AATGTTCTCATTTAAACATTACCCAGCATCATTGTTTATA |
| | | ATCAGAAACTCTGGTCCTTCTGTCTGGTGGCACTTAGAGT |
| | | CTTTTGTGCCATAATGCAGCAGTATGGAGGGAGGATTTTA |
| | | TGGAGAAATGGGGATAGTCTTCATGACCACAAATAAATA |
| | | AAGGAAAACTAAGCTGCATTGTGGGTTTTGAAAAGGTTA |
| | | TTATACTTCTTAACAATTCTTTTTTTCAGGGACTTTTCTAG |
| | | CTGTATGAC |
| SEQ ID | miR-99a | TGTATGGATTCTTTTTTCTTTTAAAACTCAATTAGAATAGT |
| NO: 22 | | TTAATTCCAAAATATTTACTATTGAAACAAAAGCAGTTCG |
| | | TGAAAAATTTTTCAATAAACTACTTTTTAAATTCTCATGC |
| | | ATATAAATTTGTATTTAGTTTTGAATATTTATGAAGGCCT |
| | | TTAATGGAGAATATGCATCCTTAGAACTCAGCATTTAAA |
| | | ACATTTATACTAAAGGATCAATATTATTTTTGACTCTTAA |
| | | TTGCATCAGATATTTACAACAAATTCTATATTAATAGGGG |
| | | GCCCATGCAAGATGTTGCCCATTGGCATAAACCCGTAGA |
| | | TCCGATCTTGTGGTGAAGTGGACCGCACAAGCTCGCTTCT |
| | | ATGGGTCTGTGTCAGTGTGGTAATCTGACAAAATGCTATA |
| | | CACAGTGCCGTTCAACAATAGTTCAGTAAAATCCTGTTAA |
| | | ACTCCAGTTGATTATATACTTTTGAAGTCATTATATTTTCC |
| | | TTTGTTTTTAATGTTTATTTTAATCATTGTCTGTCTTACAA |
| | | GGCAGGCTTCAATTCTCAACAACTTGGAAGCGTTTATATC |
| | | ACACCCATTCAAGTTCGATTCCATGTACAGTAAATTGCAT |
| | | AAGAAAGTTGAACCTTTATAGCAGGGTTTGGACCAGGAT |
| | | CTGAATAGATTCTGTTCAGAAACTTCAGTGC |
| SEQ ID | miR-150 | CGCAACCTCCTATTCCCCTCTGGGTCTCCGTCCCCTCCCTC |
| NO: 25 | | TGGAGTCCACACTCCCTCTTTGCCCCTTGCTGGTTCTCTAC |
| | | TGCCCCCAGCATAGGGTGGAGTGGGTGTGCAGTTTCTGC |
| | | GACTCAGGGTGGCGTCCCCCCAACCTGTCCCTGCCCCTTC |
| | | CTGCCCTCTTTGATGCGGCCCCACTTCCTCTGGCAGGAAC |
| | | CCCCGCCCTCCCTGGACCTGGGTATAAGGCAGGGACTGG |
| | | GCCCACGGGGAGGCAGCGTCCCCGAGGCAGCAGCGGCA |
| | | GCGGCGGCTCCTCTCCCCATGGCCCTGTCTCCCAACCCTT |
| | | GTACCAGTGCTGGGCTCAGACCCTGGTACAGGCCTGGGG |
| | | GACAGGGACCTGGGGACCCCGGCACCGGCAGGCCCCAA |
| | | GGGGTGAGGTGAGCGGGCATTGGGACCTCCCCTCCCTGT |
| | | ACTCCCATCTCTGCTGCGGCTTTTATGCGTCTCTCCCCTTC |
| | | GGGTCCCACATATCCTCTGGTGCGCTCCTGCCTCACCGCC |
| | | CCCACCCCATGCCTGTCGTCCCCACCTCTGTGTGATGCGC |
| | | AAAGTACACCTGTTTCTATTGTACCTGCCTCTCGCGGTGG |
| | | TCTGTGCTCTCCCCAGCTCTGCAAAACCCCTCCTCCCCAT |
| | | GTGCCACAACCCTGGGCCACCGTGTGTCCTGTCCTGTT |
| SEQ ID | miR-10a | CCCTTCCTCCTTTTGTGCTTAGCTAATGTTTACATCTCATA |
| NO: 28 | | ATTCATGCGCCACCGAGAGTTGCGCGGCGGCGGCGGAGG |
| | | CAAGGTTCTCGTCCCTTTGCGAACTGGCTACTTGAAATTC |
| | | TAATAGAAGAGGAGAATTGAAAACCTTGTAATCCCAAGA |
| | | ACAGACTCGCACTGCCTTTTTCTGTTCCCAGAGCTCAAAA |
| | | CTAGAACAAAACGAAATAAAACCAAAGCACTCAAACCA |
| | | CACCCCAAACGAAGAAGGCGCGGAAAGTAGGAGAACTG |
| | | GAAAATTTCTGGGCCAAGAAGATCTGTCTGTCTTCTGTAT |
| | | ATACCCTGTAGATCCGAATTTGTGTAAGGAATTTTGTGGT |
| | | CACAAATTCGTATCTAGGGGAATATGTAGTTGACATAAA |
| | | CACTCCGCTCTTATTTTTCCAGAAGAAAAAAATATATATA |
| | | TATGTATATGTAGTATTTTTCTGAATGAGGACAGTCTGGT |
| | | GACTGGCCACACGAAGACTCCTTCCTCTTTATTCCTCTAT |
| | | CTTTTCCTCCTTCAACTGGTTAGAGATGGAGAAATCATCT |
| | | ACCTGAACTCTCCCCACGCCTGCCCCTGGGAGCCTTGGTC |
| | | CCTTTTCACATCTCTTTAAGAGGTTAATGTTATTGTTGTTG |
| | | AAGTTTTAAATTTATCTTCTCGTCCCAAACGCACCCATTT |
| | | TCTATTCTGGGCTCAGGGAGGCTTTAT |
| SEQ ID | miR-95 | TGTGCCTGGATCAGGTGAGCCTTGCCGTCTGCGGCCTCCT |
| NO: 31 | | GAGGAATCCAGGCGGGAAGGGGAGCACATGGAGATTGA |
| | | GAGGGAGCCACCCTTTTTTTTCTCTCGGGGTTCTTTTGATT |
| | | GTCACCAAGTAGCCCCCAGACCTGAGTCCGTGGGCACCA |
| | | GTTCCATTTGCACACAGCTTTTACTGAACAAAGCATTTGC |
| | | ACACAGCAAGGCACGCCACCTGCACCCCGGGACGTCCAT |
| | | CTGTAGCGCGCCCAAGGAAGGTAGGATTGTGACACCCAA |
| | | CACAGTGGGCACTCAATAAATGTCTGTTGAATTGAAATG |
| | | CGTTACATTCAACGGGTATTTATTGAGCACCCACTCTGTG |
| | | CCAGACGCTGAGCGGGGCGCCGAGGGGGACAGAGAAGA |
| | | CAAGAGCAGCCCCTGACCTGGAGAAGCGTGCAGGGAGCT |
| | | GAGAGAGGCAGAGACGCCGACAGAGCGAGCGCACACGC |
| | | ATCCCGCCGCCGCCTGCCCGCCGGGCACCTCTTTGGCTTC |
| | | GCAAGGGCCCGATCACAAACTCATCTCACCCACAACACC |
| | | ATCTGCACATCTCACCCGGGACTGAGTTCATCATTCAGTC |
| | | ATTCATCCATCCCTCCACCAGATACTTACTGAGCACCTAC |
| | | TATGTGCTGGACATGG |
| SEQ ID | miR-126 | CCAACCCGACAGGTAAACAGCCCTGGCTGTGCCTGGCCT |
| NO: 34 | | GGGGAGGCGGGCAGGCAGTGGACATTGCCGTGTGGCTGT |
| | | TAGGCATGGTGGGGGGCACTGGAATCTGGGCGGAAGGCG |
| | | GTGGGGACTCCCTCTCCAGGGAGGGAGGATGGGGAGGG |
| | | AGGATAGGTGGGTTCCCGAGAACTGGGGGCAGGTTGCCC |
| | | GGAGCCTCATATCAGCCAAGAAGGCAGAAGTGCCCCGTC |
| | | CCGGGGTCCTGTCTGCATCCAGCGCAGCATTCTGGAAGA |
| | | CGCCACGCCTCCGCTGGCGACGGGACATTATTACTTTTGG |
| | | TACGCGCTGTGACACTTCAAACTCGTACCGTGAGTAATA |
| | | ATGCGCCGTCCACGGCACCGCATCGAAAACGCCGCTGAG |
| | | ACCTCAGCCTTGACCTCCCTCAGCGTGGCCGGGACCCTGA |
| | | GCCTCTGCGCAGAGCCACCCGCCCCGACGTACTTAGGCG |
| | | GCATAGCCCTGAGACCTCTGGCCAGCGCCAGGCAGGCAG |
| | | CGGGGGCGGCAGAGGCCTGGGCCTGAGTCTTCTGGCTCT |
| | | GCCTCTCCCTGGGGACAGGAGGGAGCCTGGGGGTGTGGG |
| | | TGGGGAGCCGGCCGGCCGTGACCCAGCGCCTGGCTCTGC |
| | | CCGCAGGAGTGGACAGTGCAATGAAGG |
| SEQ ID | miR-29a | ATACTACACCATTTTCTATCAGAGACTTGAGCATCTGTGG |
| NO: 37 | | ATTTTGGTATCCAAGGGGCTTTCTGGAACCAATCCCTCAA |
| | | GGATACCAAGGGATGAATGTAATTGTACAGGATATCGCA |
| | | TTGTTGGAATTTTATACTTCTTTGTGGAATAAACCTATAG |
| | | CACTTAATAGATAGTACAGACTCATTCCATTGTGCCTGGG |
| | | TTAAAGAGCCCAATGTATGCTGGATTTAGTAAGATTTGG |
| | | GCCCTCCCAACCCTCACGACCTTCTGTGACCCCTTAGAGG |
| | | ATGACTGATTTCTTTTGGTGTTCAGAGTCAATATAATTTT |
| | | CTAGCACCATCTGAAATCGGTTATAATGATTGGGGAAGA |
| | | GCACCATGATGCTGACTGCTGAGAGGAAATGTATTGGTG |
| | | ACCGTTGGGGCCATGGACAAGAACTAAGAAAACAAATGC |
| | | AAAGCAATAATGCAAAGGTGATTTTTCTTCTTCCAGTTTC |
| | | TAAGTTGAATTTCACTGACCTGAATTGCATGTGGTATAAT |
| | | ACTAACAAATGGTTCACTATTAGCATATCATGAATGGTTA |
| | | TACTTTATAGAAATTCCATAGACTTGGTGGGGGTTTTGTT |
| | | TTGGTGACGGATACCTAGAAACACTCCTGG |
| SEQ ID | miR-24 | GCACTAATCGGACATCTGTCTGAAAGGTCAAATGTATTG |
| NO: 40 | | AAAGTTGCAAAAATTCTTCTTACAAAAAACTAAAACCAA |
| | | ATGCATCACCTAAGTCGTGTGAAATCATGTGGTAGCTCAT |
| | | GGCTGTGAGCGGGGCGGGGCGGGGCTTTCGGAGGAGCTC |
| | | CTGTTGTTCTGGGCGCGGTGAACTCTCTCTTGTATTTGCA |
| | | GTCCAGGCCTTCGCGTCTCCTGCGCCAGCAGACGGTGCCC |
| | | ACGGAGCTCCCAGCTGAGGCGCTGCTTCTCCGGGCTGTC |
| | | GATTGGACCCGCCCTCCGGTGCCTACTGAGCTGATATCAG |
| | | TTCTCATTTTACACACTGGCTCAGTTCAGCAGGAACAGGA |
| | | GTCGAGCCCTTGAGCAAAAAGCCTTCGTGTCTGTAAGTG |
| | | CCCGAGGCTCAGGAGAGCTGGGGCTCCCACTCGCGGCAG |
| | | ACAGGCCCGCGTCCACCCTGCGTCCACCCCGGCCCGGCG |
| | | GCAGCACGGTGCCAGTCATCTGCATGTGCTCCTGCGGCGT |
| | | GGGGGTTTGTAGACTTGGAAAACCCTGTTGGCAGAAAGT |
| | | TAAGAGCTCCCAGGCCTGAAGGCAGGACACAGTGCCAGC |
| | | AGGGGGCCGTTTGCCCCCATGTGTAAGGGAGGCAGGGCC |
| | | CAGCTCTCCCACGGCGAGGTGA |
| SEQ ID | miR-101 | TGCTGGAAGCTTACTGCATATTTGATGTATTAGAGTGAAA |
| NO: 43 | | ACCTAATCATGCAGTTGTTCATCCTCATTAATATGGATAA |
| | | GTCATGTGTTCATCTTTCATTCTAATTTAATTCAACTGGGC |
| | | CTTTTAATATTTCAGCCTCACCACTTGCTGGGCTCTGATC |
| | | CTTCTTTTTCTTCTGCCTCCTCACGTCTCCAACCAGAAGGT |
| | | GATCTTTTAGTCCTTCACTTCATGGGGAGCCTTCAGAGAG |
| | | AGTAATGCAGCCACCAGAAAGGATGCCGTTGACCGACAC |
| | | AGTGACTGACAGGCTGCCCTGGCTCAGTTATCACAGTGCT |
| | | GATGCTGTCTATTCTAAAGGTACAGTACTGTGATAACTGA |
| | | AGGATGGCAGCCATCTTACCTTCCATCAGAGGAGCCTCA |
| | | CCGTACCCAGGAAGAAAGAAGGTGAAAGAGGAATGTGA |
| | | AACAGGTGGCTGGGACCCAGAAACCCTCTTACCCTGCAC |
| | | CTCTGTCATACTTCTCCCGGGGCATAGGGAGAGTTATTCT |
| | | GCTTCTCTTTGCCTTGTTTTGTAACATGGGGTAGTTGTTGG |
| | | TGCAGCCATGTTGTGCTGAGTGAACATATATTAAGATCTT |
| | | TGGAACCTTTAGGAGACTGAAAATAGGTAAGTATGAATT |
| | | AGTATTTCTGGAATGGTATTCAGAGAACTTCG |
| SEQ ID | miR-16 | ATCTCCTTAAAAATAATTATGCATATTACATCAATGTTAT |
| NO: 46 | | AATGTTTAAACATAGATTTTTTTACATGCATTCTTTTTTTC |
| | | CTGAAAGAAAATATTTTTTATATTCTTTAGGCGCGAATGT |
| | | GTGTTTAAAAAAAATAAAACCTTGGAGTAAAGTAGCAGC |
| | | ACATAATGGTTTGTGGATTTTGAAAAGGTGCAGGCCATA |
| | | TTGTGCTGCCTCAAAAATACAAGGATCTGATCTTCTGAAG |
| | | AAAATATATTTCTTTTTATTCATAGCTCTTATGATAGCAA |
| | | TGTCAGCAGTGCCTTAGCAGCACGTAAATATTGGCGTTA |
| | | AGATTCTAAAATTATCTCCAGTATTAACTGTGCTGCTGAA |
| | | GTAAGGTTGACCATACTCTACAGTTGTGTTTTAATGTATA |
| | | TTAATGTTACTAATGTGTTTTCAGTTTTATTGATAGTCTTT |
| | | TCAGTATTATTGATAATCTTGTTATTTTTAGTATGATTCTG |
| | | TAAAAATGAATTAATACTAATTTTTCAGATGTATCATCTC |
| | | TTAAAATACTGTAATTGCAATTTAATAATTGTATTGAATG |
| | | CCATCAAGTTTTTTTAAAAAGCTTATGCAGCATTAGAGGA |
| | | ATTTATTTTAATGCACATTTATATTCAACATAGACATTAA |
| | | TTCAGATTTTTACTTGGGA |
| SEQ ID | miR-181c | GGTAATCTGCACCTCCAGGATCACTTTGTGAATTACTTAA |
| NO: 49 | | GGAGCGGGCTTGAGGCCAGCACTCCCCTGCACTGCTACA |
| | | TCTCCATCCCCATAGCAAAGGCTACATTTTATTTACTAGC |
| | | ATTTATATTTGCCTTGTGTTTTTCCCAGATCTAGGTGCAA |
| | | ACAGCACCTGAAAAGCGTTTGTTTATTTCATGAGAGAAA |
| | | AGGGGTTCCTTACTCTCTCCTCCTCTCCCTCTTCATGCTCT |
| | | CTGGTTCCCTGCCACCTACCCCACCCCCGACTCCAGGTCC |
| | | CGGAAAATTTGCCAAGGGTTTGGGGGAACATTCAACCTG |
| | | TCGGTGAGTTTGGGCAGCTCAGGCAAACCATCGACCGTT |
| | | GAGTGGACCCTGAGGCCTGGAATTGCCATCCTCCTGCCG |
| | | GTGACTCTGACCTTCCAGATCTAGGGGGGCCTGGGGAGC |
| | | CCCCAATCCAGCCTGGGCACGTCCCCTCCCCTAGGCCACA |
| | | GCCGAGGTCACAATCAACATTCATTGTTGTCGGTGGGTTG |
| | | TGAGGACTGAGGCCAGACCCACCGGGGGATGAATGTCAC |
| | | TGTGGCTGGGCCAGACACGGCTTAAGGGGAATGGGGACT |
| | | GGGGACAGGACCCCCCACCGCCACAGTCACTCAGCCTGT |
| | | TTTTTGCCCTGACCCCAACCACTCCTCTTTGGAGAGGAGA |
| | | GCTGGTGTCTGGAATC |

### Lentivirus Production

Lentivirus particles containing plasmids of interest were created for transduction into T cells using standard methods. LentiX-293T suspension cells were transfected with the plasmids of interest along with packaging plasmids (Aldevron). DNA was mixed with OptiMEM media and Expefectamine (Invitrogen) was added. This mixture was incubated at RT for 10 minutes. After the incubation, the transfection mixture was added to the LentiX cells and incubated at 37° C. in a 220 RPM shaker for 24 hours. Expefectamine Enhancer 1 and Enhancer 2 (Invitrogen) were added to the transfected LentiX cells.

Viral supernatant was collected from 3 days post transfection. First, the cells were centrifuged at 3500 RPM for 10 minutes to clarify the viral supernatant. Next, the viral supernatant was collected, and the cell pellet was discarded. The viral supernatant was then filtered through a 0.45 μM filter and a secondary prefilter. Virus was concentrated using the Peg-IT Virus Precipitation Solution (System Biosciences). Peg-IT was added to the virus and incubated overnight at 4° C. The following day the virus/Peg-IT mixture was centrifuged at 1500×g for 1 hour. Supernatant was decanted, and the virus-containing pellet was retained. A second centrifugation step at 1500×g for 5 minutes was used to remove any excess Peg-IT solution.

The viral pellet was resuspended in 1 mL of Immunocult media, the media used to culture the T cells. Concentrated virus was then aliquoted into smaller working volumes, flash frozen in liquid nitrogen, and stored at −80° C.

### T Cell Transduction

Frozen naïve human CD4⁺ cells previously obtained via negative bead isolation (Stemcell Technologies) from peripheral blood mononuclear cells (PBMC) were thawed, washed, and rested overnight at 37° C., 5% CO₂ in Immunocult complete media (ICC). ImmunoCult-XF T expander media included: pennicillin/streptomycin, sodium pyruvate, Glutamax, NEAA, HEPES, 2-betamercaptoethanol. All cell culture experiments described herein were conducted using the ImmunoCult-XF T expander media at 37° C. 5% CO2. Cell viability and density were measured using the ViCell Blu (Beckman Coulter) and reconstituted at a concentration of 1×10⁶ cells/mL in ICC media. Activation reagent Immunocult CD3/CD28 T cell activator (Stemcell Technologies) was added to the cell suspension at a concentration of 25 ul/mL. Aliquots of cell suspension plus activation reagent were added to a multiwell plate and the cultures were activated for approximately 30 hours before lentiviral transduction.

The next day cells were transduced with different pools of lentiviral vectors at varying multiplicity of infections (MOI), Table 2, encoding human FOXP3 plus tNGFR (truncated NGFR), tNGFR alone, GFP alone, and microRNA (miR) plus GFP, all under the control of the SFFV promoter (FIGS. 2A and 2B). The viruses were prepared as a pool at the indicated MOI (Table 3) in a single lentiviral mastermix and added to the cell cultures. Viruses and virus pools were diluted in ICC media and normalized such that each culture received the same volume of lentiviral mastermix in every well.

TABLE 2
SFFV-Foxp3-tNGFR and SFFV-eGFP-
miRNA infection conditions.

| | | | Total |
|---|---|---|---|
| Condition | Virus 1 (MOI) | Virus 2 (MOI) | MOI |
| 1 | SFFV-Foxp3-tNGFR (50) | SFFV-eGFP-miRx (50) | 750 |
| 2 | SFFV-Foxp3-tNGFR (100) | SFFV-eGFP-miRx (5) | 170 |
| 3 | SFFV-Foxp3-tNGFR (50) | SFFV-eGFP (50) | 100 |
| 4 | SFFV-Foxp3-tNGFR (50) | | 100 |
| 5 | SFFV-tNGFR (50) | | 50 |

TABLE 3
SFFV-eGFP-miRNA pooled virus transduction strategy.

Condition

| | 1 | 2 |
|---|---|---|
| Virus | MOI | MOI |
| SFFV-eGFP-miR101 | 50 | 5 |
| SFFV-eGFP-miR126 | 50 | 5 |
| SFFV-eGFP-miR142 | 50 | 5 |
| SFFV-eGFP-miR146a | 50 | 5 |
| SFFV-eGFP-miR150 | 50 | 5 |
| SFFV-eGFP-miR155 | 50 | 5 |
| SFFV-eGFP-miR15b | 50 | 5 |
| SFFV-eGFP-miR16 | 50 | 5 |
| SFFV-eGFP-miR181c | 50 | 5 |
| SFFV-eGFP-miR21 | 50 | 5 |
| SFFV-eGFP-miR24 | 50 | 5 |
| SFFV-eGFP-miR29a | 50 | 5 |
| SFFV-eGFP-miR95 | 50 | 5 |
| SFFV-eGFP-miR99a | 50 | 5 |
| SFFV-FOXP3-tNGFR | 50 | 100 |

Approximately 24 hours post transduction, cells were observed via brightfield microscope to assess viability and were split into a larger well plate with ICC media plus 10 ng/mL rhIL2 (Stemcell Technologies) and left undisturbed for 55 hours.

On day 4 post transduction, cell density and viability were measured using the Vicell Blu. Cells were washed and resuspended at a cell concentration of 50×10⁶ cells/mL. Cells were stained with fluorescently labeled antibodies (Table 4) in the dark and on ice for approximately 30 min. Cells and samples for compensation were washed twice and resuspended in PBS and 5% Human AB serum at a cell concentration of 20×10⁶ cells/mL and kept in the dark on ice until ready for sorting.

TABLE 4
Reagents of flow cytometry analysis and cell sorting.

| Marker | Fluorophore | Clone | Vendor | Catalog # |
|---|---|---|---|---|
| CD127 | Percp-Cy5.5 | HIL-7R-M21 | BD Biosciences | 560551 |
| CD25 | PE | 2A3 | BD Biosciences | 341009 |
| FOXP3 | PE-CF593 | 259D/C7 | BD Biosciences | 562421 |
| CD4 | APC | RPA-T4 | BD Biosciences | 555349 |
| LIVE/DEAD | APC-e780 | n/a | ThermoFisher | 65-0865- |
| | | | | 14 |
| NGFR | BV421 | C40-1457 | BD Biosciences | 562562 |
| Trustain FC | n/a | n/a | BD Biosciences | 422302 |
| BY Buffer | n/a | n/a | BD Biosciences | 566349 |

### T cell sorting

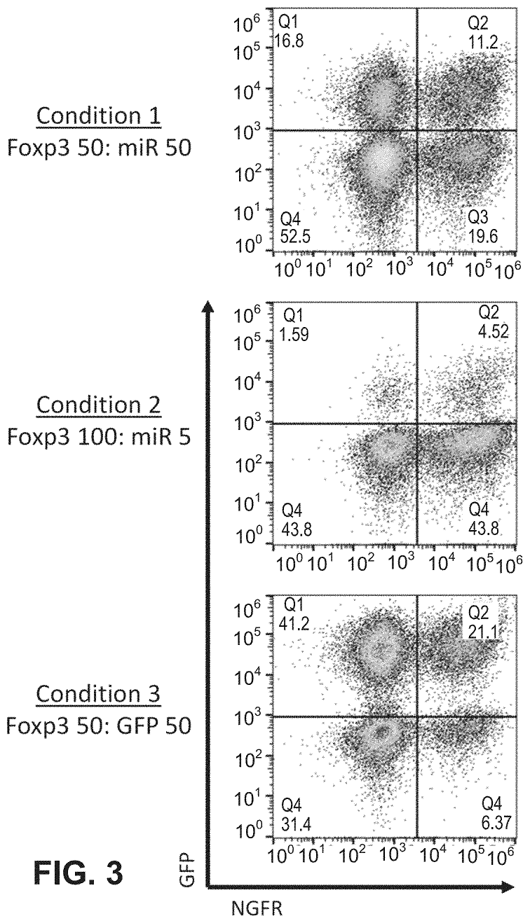

The Sony SH800 cell sorter was calibrated and setup according to manufacturer recommendations and was used for a 2-way sort of the cells based on their expression of eGFP and tNGFR (FIG. 3). Cells were sorted into a 50/50 mixture of ICC media and FBS. Two populations of cells were chosen: first, a gate sorting on GFP⁺tNGFR⁻ (FIG. 3, Quadrant 1 (Q1)) to represent cells transduced with eGFP-miR but not FOXP3-tNGFR, and secondly, a gate sorting on a GFP⁺tNGFR⁺ (FIG. 3, Quadrant 2 (Q2)) population to select cells doubly transduced with FOXP3-tNGFR and eGFP-miR.

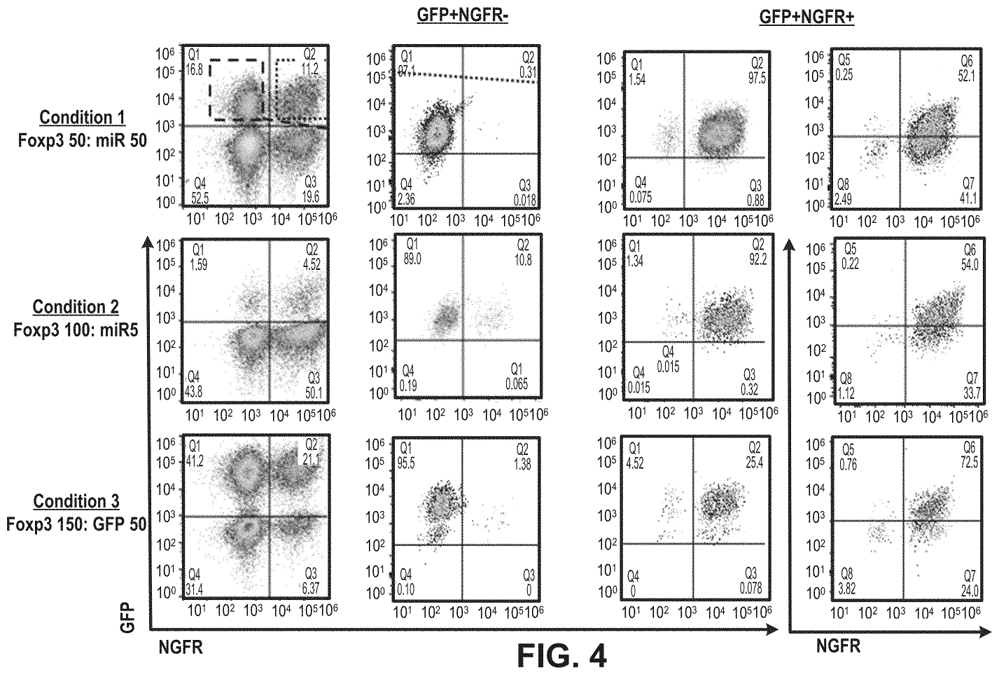

Immediately post-sort, cells were placed in the dark at 4° C. and aliquoted into cell suspensions of 50,000 cells in RPMI to be used for scRNAseq analysis, snap frozen cell pellets generated for bulk RNAseq, and fixed overnight for post sort analysis of intracellular FOXP3 expression using eBioscience Foxp3/Transcription Factor Staining Buffer set. The next day cells were permeabilized and stained for FOXP3 to validate sorting purity and expression of FOXP3 in engineered cells. Post sort analysis was collected on the Cytoflex LX, and data was analyzed with FlowJo and GraphPad Prism. Post sort analysis of purity and Foxp3 expression results show a ˜98% sorting purity for all cultures, with 57-71% FOXP3⁺ expression in the doubly transduced cells (FIG. 4).

### 10X Genomics Chromium Library Prep and scRNAseq

After T cells were sorted into populations of dual and single transduced cells, the resuspended, live cells were prepped for single cell RNA sequencing using the 10X Genomics Chromium platform. The cells were prepped for the 10X Chromium Next GEM Single Cell V(D)J Reagent Kits v1.1. Briefly, the cells were loaded onto the Chromium Next GEM Chip G along with a master mix containing Poly-dT RT primers and a reverse transcriptase, as well as other 10X proprietary additives. Gel beads and partitioning oil were loaded into separate rows of the Chip, and a 10X Gasket was attached once the Chip was fully and properly loaded with cell suspension, master mix, gel beads, and partitioning oil. Next the Chromium Chip G was loaded onto the Chromium Controller machine and a program was run that resulted in formation of GEMs (Gel Bead-in-Emulsion). Next the GEMs were run in a thermocycler under a program to allow Reverse Transcription of the captured mRNA transcripts.

Next, the GEMs underwent a clean-up and cDNA amplification using 10X proprietary master mixes. The cDNA was then analyzed on an Agilent Bioanalyzer High Sensitivity chip to determine cDNA quality and yield.

Lastly, the cDNA was constructed into a 5′ Gene Expression (GEX) Library. cDNA was fragmented, then the fragmented ends were repaired, and poly-A tailed. Next, adapters were ligated to the cDNA, and finally the resulted GEX library was analyzed on an Agilent Bioanalyzer High Sensitivity chip to confirm correct fragment size.

The resulting GEX libraries included standard Illumina paired-end barcodes. To sequence the libraries on the Illumina Miseq, the DNA was pooled, denatured, and diluted. Once the libraries were pooled and normalized to the required concentrations, they were immediately loaded into the Miseq cluster cartridge.

### Gene Expression Data Processing and Analysis

In order to detect the presence of miRs when aligning the FASTQ files with cellranger count, a custom reference package was created using cellranger mkref. For all cellranger calls, cellranger version 3.1.0 was used. Sequences of the miRs of interest were appended both to the FASTA format and GTF format of the GRCh38 human genome reference as found on the 10X genomics cellranger downloads page. The edited FASTA and GTF reference genome files were then used to create the custom reference package using cellranger mkref. This custom reference was then used as the transcriptome when aligning the FASTQ files using cellranger count.

Initial rounds of QC were performed in two ways. Using the web summary output from cellranger runs allowed important QC metrics to be checked off, and Scanpy's preprocessing library also contained important QC metrics and filtering steps.

In order to enable transcript per million (TPM) calculation for all gene counts, a function was written and applied to the counts of the anndata (annotated data matrix) object read into a jupyter notebook environment through scanpy. TPM is defined as

$$A * \frac{1}{\sum A} * {10}^{6} ,$$

where

$$A = \frac{( total reads mapped to gene ) * {10}^{3}}{gene length in base pairs} ,$$

for every gene that is in the anndata object. TPM calculation allowed for comparison between genes within a sample as well as relative comparison for genes between samples.

The presence of miRs and the genes of interest were verified through the gene_id (i.e., the customized gene_id that was specified for the miRs appended into the reference package or ensembl gene_ids for the preexisting genes in the reference package). Dictionaries were created to identify particular cells of interest which contained one or some combination of the miRs. Average counts of Treg marker genes were found for each subset of cells that contained a miR or combination of miRs and compared to global averages, which were obtained by finding the respective counts for a random sampling of 10,000 cells from the global population. Furthermore, cells containing specific miRs were inspected to determine the gene counts of genes known to be targeted by the miR.

### Results

### T Cells Transduced with Lentivirus Express Surface Markers

On day 4 post-transduction, T cells were analyzed for expression of our constructs by staining with fluorophore-conjugated antibodies to NGFR and GFP and measuring fluorescence intensity on a cytometer. Cells that stained positive for NGFR and GFP as compared to untransduced T cell controls were considered to have been transduced with both the FoxP3-NGFR and at least one GFP-miR construct. Cells transduced with varying ratios of FoxP3 to miR virus showed a range of dual positivity (FIG. 3). Cells transduced with a 1:1 ratio of FoxP3 to miR virus had 11.2% cells doubly transduced by day 4, and cells transduced with a 20:1 ratio of FoxP3 to miR virus had 4.5% cells positive for both NGFR and GFP.

### Transduced T Cells were Sorted for Dual or Single Expression of Surface Markers

After confirming cells were indeed transduced with both the FoxP3-NGFR virus and at least one GFP-miR virus, double positive cells were sorted and collected. The sorting schematic is laid out in (FIG. 4). Post sort purity analysis of the sorted T cells revealed 95-97% GFP⁺NGFR⁺ and 57-72% FoxP3⁺NGFR⁺.

### T Cells Transduced with FoxP3 Express Treg Phenotypic Markers

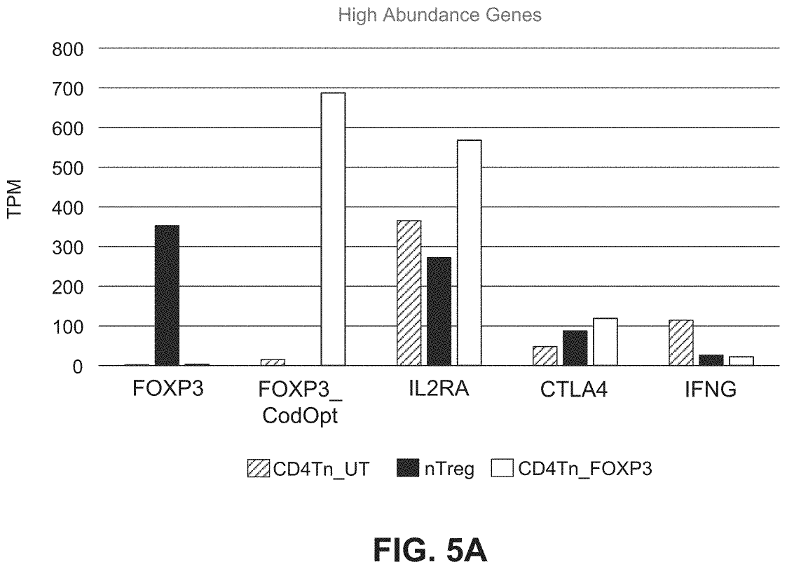

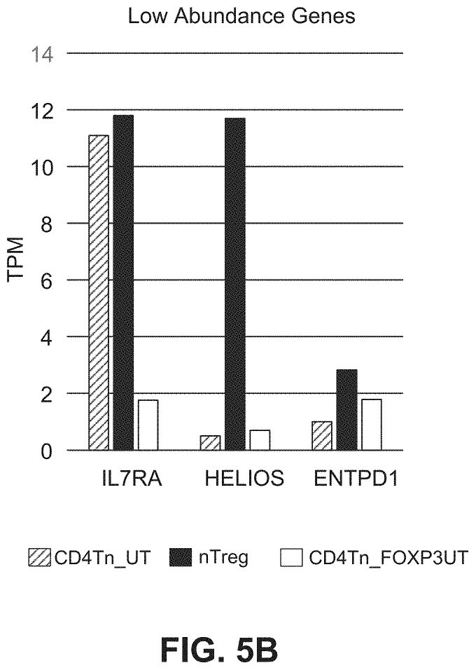

CD4⁺ naive T cells isolated from fresh human PBMCs transduced with lentivirus containing the FoxP3_tNGFR construct (hereafter referred to as “eFox” cells) (FIG. 2A) showed high levels of Treg phenotypic markers. The levels of these Treg markers were similar to or higher than natural human Tregs (nTregs) isolated from the same human PBMCs and expanded in similar conditions. Specifically, eFox cells expressed higher levels of FoxP3 and CTLA4 mRNA than either CD4⁺ naive cells or nTregs at day 4 after transduction as shown by single cell RNAseq (FIGS. 5A-5B). eFox cells expressed higher CD25 (IL2RA) than nTregs. Both nTregs and eFox cells expressed less IFNG than CD4⁺ naive T cells. eFox cells also expressed less CD127 than nTregs (FIG. 5A). One Treg phenotypic marker that was not expressed in eFox cells at nTreg levels at day 4 was Helios (FIG. 5B). CD39 (ENTPD1) was expressed in eFox cells at a higher level than in CD4⁺ naive T cells and was within 2-fold of CD39 levels in nTregs (FIG. 5B).

### T Cells Transduced with GFP miRNA Constructs Express miRNAs

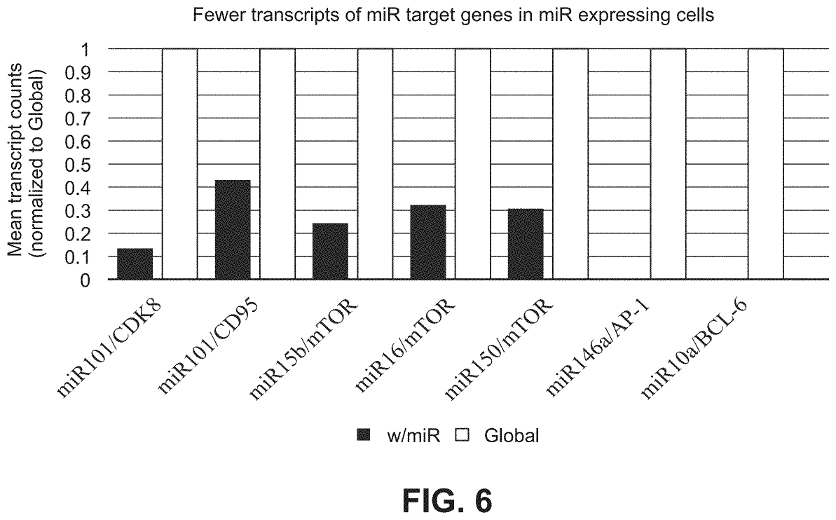

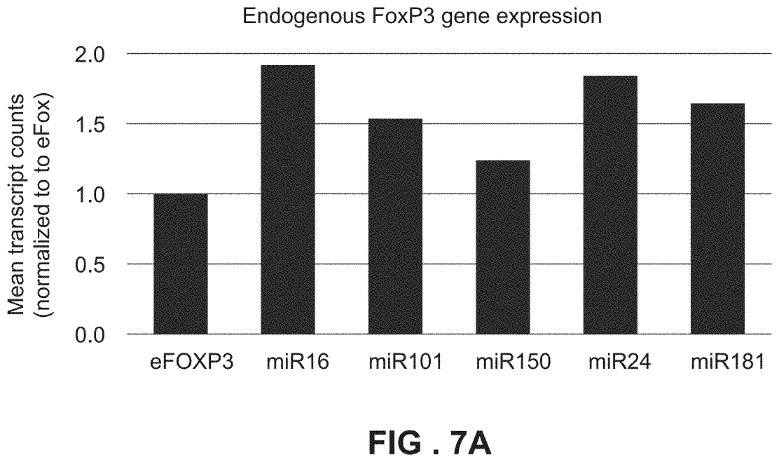

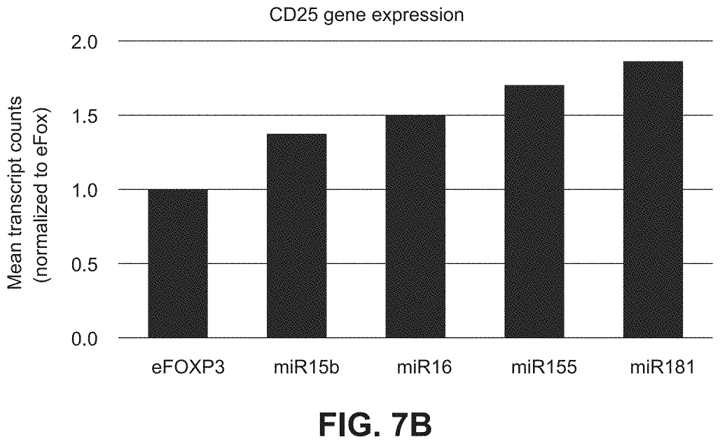

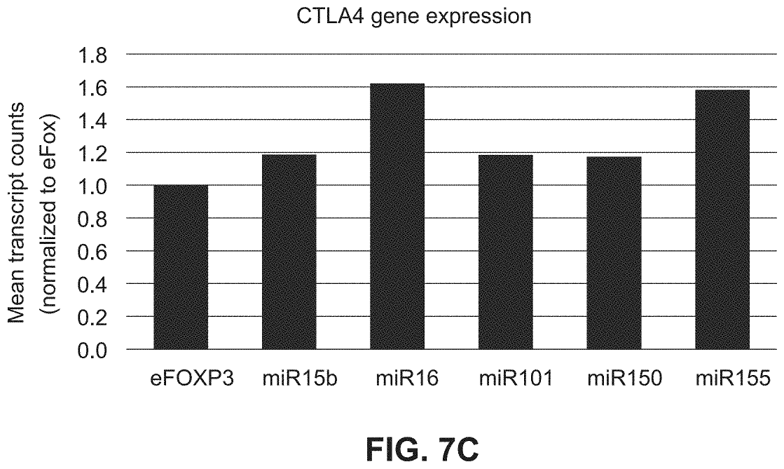

T cells transduced with a pool of GFP-miRNA lentiviruses and sorted for GFP⁺ expression express the miRNAs by day 4 post-transduction (FIG. 6 and FIG. 7). Specifically, miR101, miR15b, miR16, miR150, miR24, miR146a, miR10a, miR181c, and miR155 were detected in transduced eFox cells.

### T Cells Expressing miRNAs have Reduced Expression of miRNA Target mRNA Transcripts

T cells expressing GFP_miRNA constructs had reduced expression of miRNA target genes as compared to T cells not transduced with the same GFP_miRNA constructs (FIG. 6). Specifically, cells expressing miR101 showed 90% less expression of CDK8 and 60% less expression of CD95 than cells not transduced with a construct expressing miR101 (FIG. 6). Each miRNA that included mTOR as a target gene showed decreased mTOR levels. For example, cells expressing miR15b showed a 75% decrease in mTOR expression, cells expressing miR16 showed a 70% decrease in mTOR expression, and cells expressing miR150 showed a 70% decrease in mTOR expression, as compared to cells not transduced with the respective miRs (FIG. 6). Additional miRNA (i.e., miR146a and miR10a) abolished expression of their target genes including AP-1 and BCL-6, respectively, as compared to cells not transduced with the miRs (FIG. 6).

### T Cells Expressing FoxP3 and a miR Have Increased Expression of Treg Phenotypic Markers

eFox cells expressing a unique miRNA have higher expression of the Treg phenotypic markers FoxP3 (FIG. 7A), CD25 (FIG. 7B), and CTLA4 (FIG. 7C). Specifically, FIG. 7A shows cells expressing both eFox and miR16, miR101, miR150, miR24, or miR181c all had higher endogenous FoxP3 expression than cells expressing eFox alone. FIG. 7B shows cells expressing both eFox and miR15b, miR16, miR155, or miR181c all had higher CD25 expression than cells expressing eFox alone. Lastly, FIG. 7C shows cells expressing eFox and miR15b, miR16, miR101, miR150, or miR155 all had higher expression of CTLA4 than cells expressing eFox alone. This data shows that expressing one or more miRNA in an eFox cell (i.e., a T cell transduced with a FOXP3 polypeptide) improves the Treg phenotype (e.g., improves the immunosuppressive function) when compared to eFox cells alone or Treg cells that do not include one or more of the miRNA or the exogenous FOXP3 polypeptide.

### Example 2. miRNA-142p5 (microRNA 142) Can Be Co-Expressed with FOXP3.

A set of experiments is performed to assess the effect of co-expression of a miRNA 142-5p and a FOXP3 polypeptide. In these experiments, CD4⁺ T cells are transduced with a lentivirus where the lentiviral vector includes a first nucleic acid sequence encoding a FOXP3 polypeptide and a second nucleic acid encoding miRNA142 (e.g., miRNA 142-5p). The vector includes a SFFV promoter. Lentivirus is produced in HEK293 cells according to standard protocols.

CD4+T cells are counted and checked for viability. Next cells are resuspended in fresh serum free, for example ImmunoCult T cell expansion media, at a concentration of 10⁶ cells/mL. Then 500 μL (˜500,000 cells) of a cell suspension is aliquoted to each well. The cells are then cultured in the presence of CD3/CD28 for 1-2 days prior to addition of virus. Different concentrations of lentiviral particles are added to each well for the desired target MOI. The plates are then sealed with parafilm, and the cells are spun in a table top centrifuge at 300×g for 5 minutes. After spinoculation, the cells are incubated at 37° C.

miRNA-142p5 is detected by real-time qPCR, using a miRNA-specific stem-loop primer and subsequent amplification. This is done using the TaqMan Advanced miRNA cDNA Synthesis Kit (Thermo Fisher), which is a qPCR-based method used to quantify specific miRNA species in a sample. In brief, cDNA synthesis is followed by a fluorescent detection step using a probe specific for the target miRNA. Due to its short length, the mature miRNA must first be elongated by the cDNA synthesis step before qPCR detection can be performed. In the cDNA synthesis step, the target miRNA is primed with a poly(A) tailing reaction catalyzed by poly-A polymerase. This reaction is followed by an adapter ligation reaction in which an adaptor is ligated to the miRNA-polyA construct. The target sequence is then reverse transcribed using a universal hair-pin primer and amplified by a DNA polymerase. The target sequence is then detected and quantified using a fluorescent probe specific for the target miRNA sequence.

### Example 3. T Cells Transduced with FOXP3 and miRNAs Show Increased Growth and Survival Advantage

A set of experiments was performed to assess the effect growth and survival advantage of activated T-cells transduced with a lentivirus where the lentiviral vector includes a first nucleic acid sequence encoding a FOXP3 polypeptide and a second nucleic acid encoding one of miR-10a, miR-95, miR-29a, miR-150, or miR-101. A control vector encoding a FOXP3 polypeptide and GFP was used as a control for expression. The vector includes a SFFV promoter.

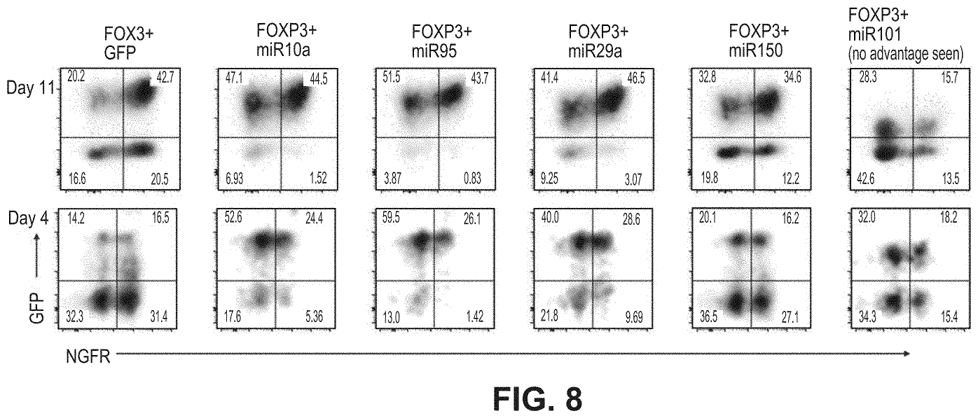

Naïve CD4 T cells were activated with plate-bound anti-CD3 antibody (clone UCHT1, 10 ug/mL) for 24 hours on non-tissue culture treated plastic. Activated cells were co-transduced with 2 lentiviruses: SFFV-FOXP3-tNGFR and SSFV-miRNA-GFP. Transduced cells were expanded in the presence of rhIL-2 and re-activated at day 8 post-transduction with ImmunoCult Human CD3/CD28 T Cell Activator. Samples were taken to assess transduction frequency by flow cytometry at days 4 and 11 post-transduction. Briefly, samples were stained with anti-NGFR as a readout of FOXP3 transduction. GFP signal was detected as a proxy for miRNA expression. Cells were pre-gated as live CD4+ lymphocytes. FIG. 8 shows enrichment of double-positive cells and a lack of double-negative cells between day 4 and day 11, thus showing increased growth and survival advantage in activated T cells expressing a FOX3 polypeptide and one of miR-10a, miR-95, miR-29a, or miR-150. No advantage was observed for miR-101.

### Example 4. T-Cells Transduced with FOXP3 and miR-155 Show Increased Survival, IL-2 Sensitivity, and Expansion

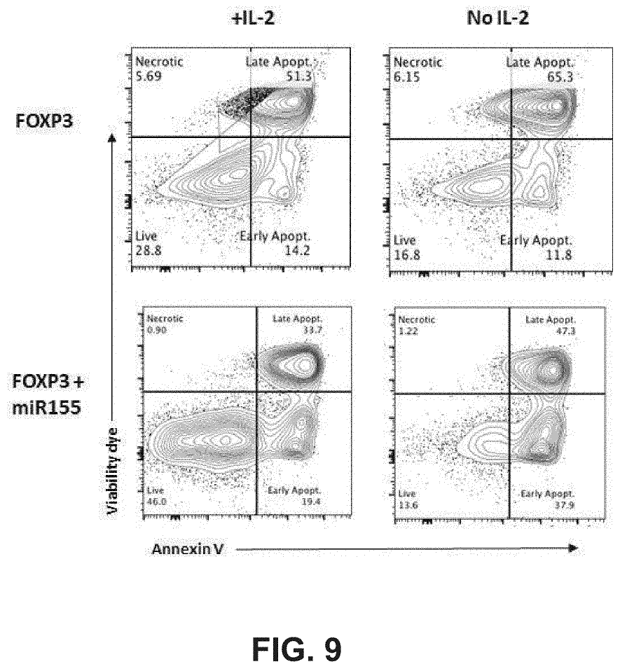

A set of experiments was performed to assess the survival, IL-2 sensitivity, and expansion of activated T cells (as described in Example 3) transduced with a lentivirus where the lentiviral vector includes a first nucleic acid encoding a FOXP3 polypeptide and a second nucleic acid encoding miR-155. FIG. 9 shows four panels of fluorescence activated cell sorting (FACS) to assess viability under different conditions. Cells were stained with a viability dye (e.g., live cells) and Annexin V, which is commonly used in cell sorting to detect apoptotic cells based on its ability to bind phosphatidylserine, a marker of apoptosis. The activated T cells in the left-hand panels were also contacted with IL-2, whereas the panels on the right-hand side were not. The panels shown on the top were only contacted with a nucleic acid encoding a FOXP3 polypeptide, whereas the panels shown on the bottom were contacted with both a nucleic acid encoding a FOXP3 polypeptide and a second nucleic acid encoding miR-155.

The data demonstrate that activated T-cells contacted with both a nucleic acid encoding a FOXP3 polypeptide and a second nucleic acid encoding miR-155 and IL-2 (bottom left panel) showed increased number of live cells (46%) relative to all other conditions tested as measured by FACS analysis (Top left panel: FOXP3 and IL-2 (28.8%); Top right panel: FOXP3 and no IL-2 (16.8%); Bottom right panel: FOXP3 and miR-155 and no IL-2 (13.6%). In addition, activated T-cells contacted with both a nucleic acid encoding a FOXP3 polypeptide and a second nucleic acid encoding miR-155 and IL-2 (bottom left panel) showed reduced necrotic, early apoptotic and late apoptotic cells. The data is summarized in Table 5 below.

TABLE 5

| | Live | Necrotic | Early | Late |
|---|---|---|---|---|
| | (%) | (%) | Apoptotic (%) | Apoptotic (%) |
| FOXP3 | 16.8 | 6.15 | 11.8 | 65.3 |
| FOXP3 + IL-2 | 28.8 | 5.69 | 14.2 | 51.3 |
| FOXP3 + miR- | 13.6 | 1.22 | 37.9 | 47.3 |
| 155 | | | | |
| FOXP3 + miR- | 46.0 | 0.90 | 19.4 | 33.7 |
| 155 + IL-2 | | | | |

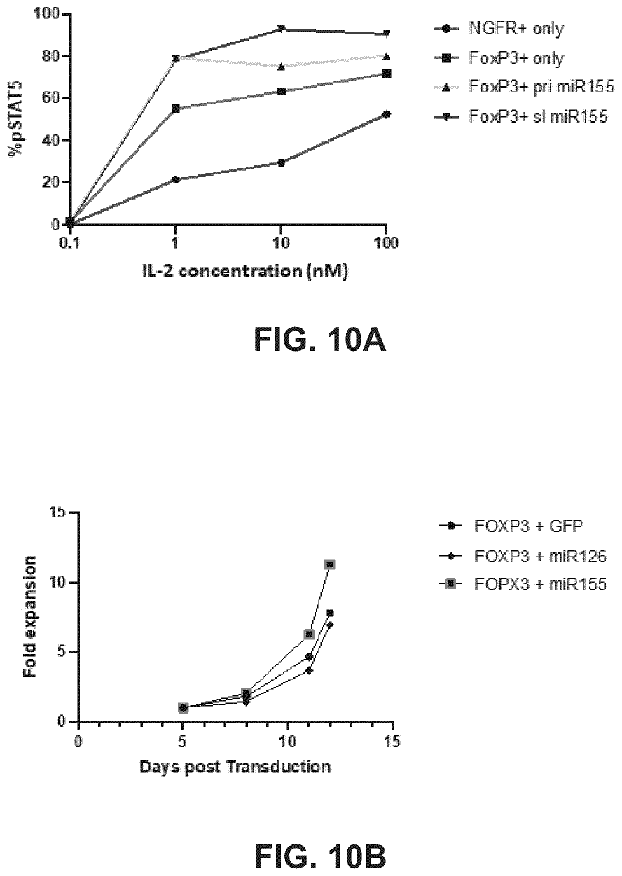

FIG. 10A is a graph showing the percent phosopho-STAT5 (pSTAT5) measured on activated T cells under increasing concentrations of IL-2 (0.1 mM, 1.0 mM, 10 mM, and 100 mM) transduced with different nucleic acids (Nerve Growth Factor (NFGR), FOXP3 alone, FOXP3+primary (pri) miR-155, and FOXP3+stem loop (sl) miR-155). Assessing pSTAT5 levels measures IL-2-inducement and can reveal the cytokine responsiveness of individual T cells. As shown in FIG. 10A activated T cells transduced with either FOXP3 and pri miR-155 or FOXP3 and sl miR-155 show greater levels of pSTAT5 than either FOXP3 or NGFR alone. FIG. 10B shows the fold expansion of activated T cells at different days post transduction (day 5, day 8, day 11, and day 12). The activated T cells were transduced with either FOXP3 and GFP (control), FOXP3 and miR-126, or FOXP3 and miR-155. The data show that activated T cells transduced with FOXP3 and miR-155 had increased fold expansion relative to FOXP3 and GFP and FOXP3 and miR-126.

Collectively, the data in FIG. 9 and FIGS. 10A-B demonstrate that activated T cells transduced with both a nucleic acid encoding a FOXP3 polypeptide and a nucleic acid encoding miR-155 lead to increased survival, IL-2 sensitivity, and expansion of the T cells.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for increasing T cell function, wherein the method comprises introducing into a T cell:
(i) a first nucleic acid sequence encoding a FOXP3 polypeptide; and
(ii) a second nucleic acid sequence encoding a microRNA.

2. The method of claim 1, wherein the FOXP3 polypeptide comprises a sequence that is at least 80% identical to SEQ ID NO: 1.

3. (canceled)

4. (canceled)

5. (canceled)

6. The method of claim 1, wherein the microRNA is selected form the group consisting of: miR-142, miR-155, miR-15b, miR-16, miR-146a, miR-21a, miR-99a, miR-150, miR-10a, miR-95, miR-126, miR-29a, miR-24, miR-181c, and miR-101.

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. The method of claim 1, wherein the T cell, after the introducing, has one or more of the following activities: increased levels of FoxP3 mRNA and/or FoxP3 protein, increased levels of CD25 mRNA and/or CD25 protein, and increased levels of CTLA4 mRNA and/or CTLA4 protein, as compared to a T cell including the first nucleic acid, but not including the second nucleic acid.

23. The method of claim 1, wherein the first nucleic acid sequence further comprises a nucleic acid sequence encoding a truncated nerve growth factor receptor (tNGFR) polypeptide.

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. The method of claim 1, wherein the T cell is a CD4⁺ T cell or a CD4⁺/CD45RA⁺ T cell.

38. The method of claim 1, wherein the method further comprises:
obtaining a T cell from a patient or obtaining T cells allogenic to the patient.

39. The method of claim 38, wherein the method further comprises:
treating the obtained T cells to isolate a population of cells enriched for CD4⁺ T cells or CD4⁺/CD45RA⁺ T cells.

40. A T cell produced by the method of claim 1.

41. A composition comprising the T cell of claim 40.

42. A T-cell comprising:
a first nucleic acid sequence encoding a FOXP3 polypeptide; and
a second nucleic acid sequence encoding a microRNA.

43. The T-cell of claim 42, wherein the FOXP3 polypeptide comprises a sequence that is at least 80% identical to SEQ ID NO: 1.

44. (canceled)

45. (canceled)

46. (canceled)

47. The T-cell of claim 42, wherein the microRNA is selected form the group consisting of: miR-142, miR-155, miR-15b, miR-16, miR-146a, miR-21a, miR-99a, miR-150, miR-10a, miR-95, miR-126, miR-29a, miR-24, miR-181c, and miR-101.

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. The T-cell of claim 42, wherein the T cell, after the introducing, has one or more of the following activities: increased levels of FoxP3 mRNA and/or FoxP3 protein, increased levels of CD25 mRNA and/or CD25 protein, and increased levels of CTLA4 mRNA and/or CTLA4 protein, as compared to a T cell including the first nucleic acid, but not including the second nucleic acid.

64. The T-cell of claim 42, wherein the first nucleic acid sequence further comprises a nucleic acid sequence encoding a truncated nerve growth factor receptor (tNGFR) polypeptide.

65. (canceled)

66. (canceled)

67. (canceled)

68. (canceled)

69. (canceled)

70. A composition comprising a T cell of claim 42.

71. A method of producing a T cell population expressing an exogenous FOXP3 polypeptide and a microRNA, the method comprising culturing a T cell of claim 42 in growth media under conditions sufficient to expand the population of T cells.

72. A population of T cells prepared by the method of claim 71.

73. A composition comprising the population of T cells of claim 72.

74. A vector comprising a first nucleic acid sequence encoding a FOXP3 polypeptide and a second nucleic acid sequence encoding a micro-RNA.

75. The vector of claim 74, wherein the FOXP3 polypeptide comprises a sequence that is at least 80% identical to SEQ ID NO: 1.

76. (canceled)

77. (canceled)

78. (canceled)

79. The vector of claim 74, wherein the microRNA is selected form the group consisting of: miR-142, miR-155, miR-15b, miR-16, miR-146a, miR-21a, miR-99a, miR-150, miR-10a, miR-95, miR-126, miR-29a, miR-24, miR-181c, and miR-101.

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. The vector of claim 74, wherein the first nucleic acid sequence further comprises a nucleic acid sequence encoding a truncated nerve growth factor receptor (tNGFR) polypeptide.

96. (canceled)

97. (canceled)

98. (canceled)

99. (canceled)

100. (canceled)

101. (canceled)

102. (canceled)

103. (canceled)

104. (canceled)

105. (canceled)

106. (canceled)

107. A composition comprising the vector of claim 74.

108. A kit comprising the composition of claim 70.

109. A method of treating an autoimmune disease or disorder in a patient comprising administering a T cell of claim 42.

110. (canceled)

111. (canceled)

112. (canceled)

113. (canceled)


SEQUENCE LISTING (53 sequences)

SEQ ID NO: 1 - protein, 431 aa, Homo sapiens
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 2 - DNA, 1293 bp, Homo sapiens
atgcctaacccccgccctggaaaaccatctgccccttcactggccctgggaccttcacccggagcctcaccatcttggagagccgcccccaaggccagcgacctgctgggagccagaggccccggcggcaccttccagggcagggatctgcgcggcggcgcccacgccagctcctctagcctgaaccccatgcccccttctcagctccagctgcccacactgcccctggtcatggtggcacctagcggagcaaggctgggaccactgccacacctccaggccctgctccaggacagacctcactttatgcaccagctgtccaccgtggatgcacacgcaaggacacccgtgctccaggtgcaccctctggagtctccagccatgatcagcctgaccccaccaaccacagcaacaggcgtgttctccctgaaggccagacctggcctgcctccaggcatcaacgtggcctccctggagtgggtgtctagggagccagccctgctgtgcacctttcctaatccatctgccccccgcaaggactccacactgtctgccgtgccacagtcctcttaccccctgctggccaacggcgtgtgcaagtggcctggctgtgagaaggtgttcgaggagccagaggattttctgaagcactgccaggccgaccacctgctggatgagaagggaagggcacagtgtctgctccagagggagatggtgcagagcctggagcagcagctggtgctggagaaggagaagctgtccgccatgcaggcacacctggcaggcaagatggcactgaccaaggccagctccgtggcctctagcgacaagggcagctgctgtatcgtggccgccggctcccagggaccagtggtgcccgcctggtctggacccagggaggcacctgacagcctgttcgccgtgcggagacacctgtggggcagccacggcaattccaccttccccgagtttctgcacaacatggattacttcaagtttcacaatatgcggcccccttttacctatgccacactgatcagatgggccatcctggaggccccagagaagcagcgcaccctgaacgaaatctaccactggttcacacggatgtttgccttctttagaaatcaccccgccacctggaagaacgccatcaggcacaatctgtccctgcacaagtgtttcgtgcgcgtggagtctgagaagggcgccgtgtggacagtggatgagctggagttcagaaagaagagaagccagagaccatccaggtgttcaaaccctaccccaggaccc

SEQ ID NO: 3 - RNA, 21 nt, Homo sapiens
cauaaaguagaaagcacuacu

SEQ ID NO: 4 - RNA, 87 nt, Homo sapiens
gacagugcagucacccauaaaguagaaagcacuacuaacagcacuggaggguguaguguuuccuacuuuauggaugaguguacugug

SEQ ID NO: 5 - DNA, 655 bp, Homo sapiens
cctagtctctacctgagtgtctctgaaactggggggatggggtggagcctttagggggaagggaagagggaactgaagaggaagtgggggagggaggtagaggaggcaagtctggcgccatgctgagtcaccgcccacaaggcccagggcgggccctcggggggccctggcagggttggggggatcttaggaagccacaaggagggctggggggctcttggagcaggagtcaggaggcctgggcagcctgaagagtacacgccgacggacagacagacagtgcagtcacccataaagtagaaagcactactaacagcactggagggtgtagtgtttcctactttatggatgagtgtactgtgggcttcggagatcacgccactgctgccgcccgctgcccgccaccatcttcctcggcgctcggggacctcgtgtgacaggtgagcaccttacggcccctccctaccctgcccagatgcctgaaaggcctccatggctttcctgcccttcctggttccggacagctggggaaaggccacagcagctcctctgctgccctgcagtctttgggggcggggagggctggacatgtggaaccctgatgcagccgcagcgtcaaggacgaggaaggggtgggaagggatggtacgtggag

SEQ ID NO: 6 - RNA, 24 nt, Homo sapiens
uuaaugcuaaucgugauagggguu

SEQ ID NO: 7 - RNA, 65 nt, Homo sapiens
cuguuaaugcuaaucgugauagggguuuuugccuccaacugacuccuacauauuagcauuaacag

SEQ ID NO: 8 - DNA, 644 bp, Homo sapiens
tatgcctaaaggtaacaatgtcatcttttaattgccaatttctctaccactttcaaaaaattacttccaaggatttaatgagctccttcctttcaacagaaaatggactattttcctttcagatttactatatgctgtcactccagctttataaccgcatgtgcatacacaaacatttctttctctcttgcaggtggcacaaaccaggaaggggaaatctgtggtttaaattctttatgcctcatcctctgagtgctgaaggcttgctgtaggctgtatgctgttaatgctaatcgtgataggggtttttgcctccaactgactcctacatattagcattaacagtgtatgatgcctgttactagcattcacatggaacaaattgctgccgtgggaggatgacaaagaagcatgagtcaccctgctggataaacttagacttcaggctttatcatttttcaatctgttaatcataatctggtcactgggatgttcaaccttaaactaagttttgaaagtaaggttatttaaaagatttatcagtagtatcctaaatgcaaacattttcatttaaatgtcaagcccatgtttgtttttatcattaacagaaaatatattcatgtcattcttaattgcaggttttg

SEQ ID NO: 9 - RNA, 22 nt, Homo sapiens
uagcagcacaucaugguuuaca

SEQ ID NO: 10 - RNA, 98 nt, Homo sapiens
uugaggccuuaaaguacuguagcagcacaucaugguuuacaugcuacagucaagaugcgaaucauuauuugcugcucuagaaauuuaaggaaauucau

SEQ ID NO: 11 - DNA, 653 bp, Homo sapiens
ttttcctcaaaacaggaaggggatgattatgaagtcattcctaacagtaatttctatgtatccagaacggcctgcagagataatacttctgtctatcacataagtggaaagaaaaagacatttaaggatgttggaaatcttcttcgaagccatggaattgacttggaccataatagatttttaattttacaggtaagtttattaaagacttcaaagattctcttattcttgttacttttttttctataaagctaggttggatgaatcctacatttttgaggccttaaagtactgtagcagcacatcatggtttacatgctacagtcaagatgcgaatcattatttgctgctctagaaatttaaggaaattcattcaaaactatgttttcatcatcagatgttcgttttatgtttggatgaactgacatacttgttccactctagcagcacgtaaatattggcgtagtgaaatatatattaaacaccaatattactgtgctgctttagtgtgacagggatacagcaactattttatcaattgtttgtatttccctttaaggtaacattttaaatgaaatgtattatattttaatctatccttttcctttgtttttgttcttattatctcttctgatatataaccaaaaaatgaa

SEQ ID NO: 12 - RNA, 22 nt, Homo sapiens
ugagaacugaauuccauggguu

SEQ ID NO: 13 - RNA, 99 nt, Homo sapiens
ccgauguguauccucagcuuugagaacugaauuccauggguugugucagugucagaccucugaaauucaguucuucagcugggauaucucugucaucgu

SEQ ID NO: 14 - DNA, 685 bp, Homo sapiens
tcaagcgatcctcccaccacaggccatcatgcatggctcattttttatttttagtagagacaaattctccatgttgcccaggctagtcctgaactcctgggctcaagagatccacccacatcagccttccagactgctggcctggtctcctccagatgtttataactcatgagtgccaggactagacctggtactaggaagcagctgcattggatttaccaggcttttcactcttgtattttacagggctgggacaggcctggactgcaaggaggggtctttgcaccatctctgaaaagccgatgtgtatcctcagctttgagaactgaattccatgggttgtgtcagtgtcagacctctgaaattcagttcttcagctgggatatctctgtcatcgtgggcttgaggacctggagagagtagatcctgaagaactttttcagtctgctgaagagcttggaagactggagacagaaggcagagtctcaggctctgaaggtataaggagtgtgagttcctgtgagaaacactcatttgattgtgaaaagacttgaattctatgctaagcagggttccaagtagctaaatgaatgatctcagcaagtctctcttgctgctgctgctactcgtttacatttattgattacttacgatgattcaggtactgttgtaagtgctttacatg

SEQ ID NO: 15 - RNA, 23 nt, Homo sapiens
ugagaacugaauuccauaggcug

SEQ ID NO: 16 - RNA, 73 nt, Homo sapiens
ccuggcacugagaacugaauuccauaggcugugagcucuagcaaugcccuguggacucaguucuggugcccgg

SEQ ID NO: 17 - RNA, 22 nt, Homo sapiens
uagcuuaucagacugauguuga

SEQ ID NO: 18 - RNA, 92 nt, Homo sapiens
uguaccaccuugucggauagcuuaucagacugauguugacuguugaaucucauggcaacagcagucgaugggcugucugacauuuugguauc

SEQ ID NO: 19 - DNA, 651 bp, Homo sapiens
cagttttcttgccgttctgtaagtgttttattcttagtgtgatttttttccattgggatgtttttgattgaacttgttcattttgttttgcttgggaggaaaataaacaattttacttttttcctttaggagcattatgagcattatgtcagaatagaatagaattggggttcgatcttaacaggccagaaatgcctgggtttttttggtttgtttttgtttttgtttttttatcaaatcctgcctgactgtctgcttgttttgcctaccatcgtgacatctccatggctgtaccaccttgtcgggtagcttatcagactgatgttgactgttgaatctcatggcaacaccagtcgatgggctgtctgacattttggtatctttcatctgaccatccatatccaatgttctcatttaaacattacccagcatcattgtttataatcagaaactctggtccttctgtctggtggcacttagagtcttttgtgccataatgcagcagtatggagggaggattttatggagaaatggggatagtcttcatgaccacaaataaataaaggaaaactaagctgcattgtgggttttgaaaaggttattatacttcttaacaattctttttttcagggacttttctagctgtatgac

SEQ ID NO: 20 - RNA, 22 nt, Homo sapiens
aacccguagauccgaucuugug

SEQ ID NO: 21 - RNA, 81 nt, Homo sapiens
cccauuggcauaaacccguagauccgaucuuguggugaaguggaccgcacaagcucgcuucuaugggucugugucagugug

SEQ ID NO: 22 - DNA, 671 bp, Homo sapiens
tgtatggattcttttttcttttaaaactcaattagaatagtttaattccaaaatatttactattgaaacaaaagcagttcgtgaaaaatttttcaataaactactttttaaattctcatgcatataaatttgtatttagttttgaatatttatgaaggcctttaatggagaatatgcatccttagaactcagcatttaaaacatttatactaaaggatcaatattatttttgactcttaattgcatcagatatttacaacaaattctatattaatagggggcccatgcaagatgttgcccattggcataaacccgtagatccgatcttgtggtgaagtggaccgcacaagctcgcttctatgggtctgtgtcagtgtggtaatctgacaaaatgctatacacagtgccgttcaacaatagttcagtaaaatcctgttaaactccagttgattatatacttttgaagtcattatattttcctttgtttttaatgtttattttaatcattgtctgtcttacaaggcaggcttcaattctcaacaacttggaagcgtttatatcacacccattcaagttcgattccatgtacagtaaattgcataagaaagttgaacctttatagcagggtttggaccaggatctgaatagattctgttcagaaacttcagtgc

SEQ ID NO: 23 - RNA, 22 nt, Homo sapiens
ucucccaacccuuguaccagug

SEQ ID NO: 24 - RNA, 84 nt, Homo sapiens
cuccccauggcccugucucccaacccuuguaccagugcugggcucagacccugguacaggccugggggacagggaccuggggac

SEQ ID NO: 25 - DNA, 673 bp, Homo sapiens
cgcaacctcctattcccctctgggtctccgtcccctccctctggagtccacactccctctttgccccttgctggttctctactgcccccagcatagggtggagtgggtgtgcagtttctgcgactcagggtggcgtccccccaacctgtccctgccccttcctgccctctttgatgcggccccacttcctctggcaggaacccccgccctccctggacctgggtataaggcagggactgggcccacggggaggcagcgtccccgaggcagcagcggcagcggcggctcctctccccatggccctgtctcccaacccttgtaccagtgctgggctcagaccctggtacaggcctgggggacagggacctggggaccccggcaccggcaggccccaaggggtgaggtgagcgggcattgggacctcccctccctgtactcccatctctgctgcggcttttatgcgtctctccccttcgggtcccacatatcctctggtgcgctcctgcctcaccgcccccaccccatgcctgtcgtccccacctctgtgtgatgcgcaaagtacacctgtttctattgtacctgcctctcgcggtggtctgtgctctccccagctctgcaaaacccctcctccccatgtgccacaaccctgggccaccgtgtgtcctgtcctgtt

SEQ ID NO: 26 - RNA, 23 nt, Homo sapiens
uacccuguagauccgaauuugug

SEQ ID NO: 27 - RNA, 110 nt, Homo sapiens
gaucugucugucuucuguauauacccuguagauccgaauuuguguaaggaauuuuguggucacaaauucguaucuaggggaauauguaguugacauaaacacuccgcucu

SEQ ID NO: 28 - DNA, 702 bp, Homo sapiens
cccttcctccttttgtgcttagctaatgtttacatctcataattcatgcgccaccgagagttgcgcggcggcggcggaggcaaggttctcgtccctttgcgaactggctacttgaaattctaatagaagaggagaattgaaaaccttgtaatcccaagaacagactcgcactgcctttttctgttcccagagctcaaaactagaacaaaacgaaataaaaccaaagcactcaaaccacaccccaaacgaagaaggcgcggaaagtaggagaactggaaaatttctgggccaagaagatctgtctgtcttctgtatataccctgtagatccgaatttgtgtaaggaattttgtggtcacaaattcgtatctaggggaatatgtagttgacataaacactccgctcttatttttccagaagaaaaaaatatatatatatgtatatgtagtatttttctgaatgaggacagtctggtgactggccacacgaagactccttcctctttattcctctatcttttcctccttcaactggttagagatggagaaatcatctacctgaactctccccacgcctgcccctgggagccttggtcccttttcacatctctttaagaggttaatgttattgttgttgaagttttaaatttatcttctcgtcccaaacgcacccattttctattctgggctcagggaggctttat

SEQ ID NO: 29 - RNA, 21 nt, Homo sapiens
ucaauaaaugucuguugaauu

SEQ ID NO: 30 - RNA, 81 nt, Homo sapiens
aacacagugggcacucaauaaaugucuguugaauugaaaugcguuacauucaacggguauuuauugagcacccacucugug

SEQ ID NO: 31 - DNA, 645 bp, Homo sapiens
tgtgcctggatcaggtgagccttgccgtctgcggcctcctgaggaatccaggcgggaaggggagcacatggagattgagagggagccacccttttttttctctcggggttcttttgattgtcaccaagtagcccccagacctgagtccgtgggcaccagttccatttgcacacagcttttactgaacaaagcatttgcacacagcaaggcacgccacctgcaccccgggacgtccatctgtagcgcgcccaaggaaggtaggattgtgacacccaacacagtgggcactcaataaatgtctgttgaattgaaatgcgttacattcaacgggtatttattgagcacccactctgtgccagacgctgagcggggcgccgagggggacagagaagacaagagcagcccctgacctggagaagcgtgcagggagctgagagaggcagagacgccgacagagcgagcgcacacgcatcccgccgccgcctgcccgccgggcacctctttggcttcgcaagggcccgatcacaaactcatctcacccacaacaccatctgcacatctcacccgggactgagttcatcattcagtcattcatccatccctccaccagatacttactgagcacctactatgtgctggacatgg

SEQ ID NO: 32 - RNA, 21 nt, Homo sapiens
cauuauuacuuuugguacgcg

SEQ ID NO: 33 - RNA, 84 nt, Homo sapiens
gcuggcgacgggacauuauuacuuuugguacgcgcugugacacuucaaacucguaccgugaguaauaaugcgccguccacggca

SEQ ID NO: 34 - DNA, 652 bp, Homo sapiens
ccaacccgacaggtaaacagccctggctgtgcctggcctggggaggcgggcaggcagtggacattgccgtgtggctgttaggcatggtggggggcactggaatctgggcggaaggcggtggggactccctctccagggagggaggatggggagggaggataggtgggttcccgagaactgggggcaggttgcccggagcctcatatcagccaagaaggcagaagtgccccgtcccggggtcctgtctgcatccagcgcagcattctggaagacgccacgcctccgctggcgacgggacattattacttttggtacgcgctgtgacacttcaaactcgtaccgtgagtaataatgcgccgtccacggcaccgcatcgaaaacgccgctgagacctcagccttgacctccctcagcgtggccgggaccctgagcctctgcgcagagccacccgccccgacgtacttaggcggcatagccctgagacctctggccagcgccaggcaggcagcgggggcggcagaggcctgggcctgagtcttctggctctgcctctccctggggacaggagggagcctgggggtgtgggtggggagccggccggccgtgacccagcgcctggctctgcccgcaggagtggacagtgcaatgaagg

SEQ ID NO: 35 - RNA, 22 nt, Homo sapiens
acugauuucuuuugguguucag

SEQ ID NO: 36 - RNA, 64 nt, Homo sapiens
augacugauuucuuuugguguucagagucaauauaauuuucuagcaccaucugaaaucgguuau

SEQ ID NO: 37 - DNA, 625 bp, Homo sapiens
atactacaccattttctatcagagacttgagcatctgtggattttggtatccaaggggctttctggaaccaatccctcaaggataccaagggatgaatgtaattgtacaggatatcgcattgttggaattttatacttctttgtggaataaacctatagcacttaatagatagtacagactcattccattgtgcctgggttaaagagcccaatgtatgctggatttagtaagatttgggccctcccaaccctcacgaccttctgtgaccccttagaggatgactgatttcttttggtgttcagagtcaatataattttctagcaccatctgaaatcggttataatgattggggaagagcaccatgatgctgactgctgagaggaaatgtattggtgaccgttggggccatggacaagaactaagaaaacaaatgcaaagcaataatgcaaaggtgatttttcttcttccagtttctaagttgaatttcactgacctgaattgcatgtggtataatactaacaaatggttcactattagcatatcatgaatggttatactttatagaaattccatagacttggtgggggttttgttttggtgacggatacctagaaacactcctgg

SEQ ID NO: 38 - RNA, 21 nt, Homo sapiens
cauaaaguagaaagcacuacu

SEQ ID NO: 39 - RNA, 73 nt, Homo sapiens
cucugccucccgugccuacugagcugaaacacaguugguuuguguacacuggcucaguucagcaggaacaggg

SEQ ID NO: 40 - DNA, 652 bp, Homo sapiens
gcactaatcggacatctgtctgaaaggtcaaatgtattgaaagttgcaaaaattcttcttacaaaaaactaaaaccaaatgcatcacctaagtcgtgtgaaatcatgtggtagctcatggctgtgagcggggcggggcggggctttcggaggagctcctgttgttctgggcgcggtgaactctctcttgtatttgcagtccaggccttcgcgtctcctgcgccagcagacggtgcccacggagctcccagctgaggcgctgcttctccgggctgtcgattggacccgccctccggtgcctactgagctgatatcagttctcattttacacactggctcagttcagcaggaacaggagtcgagcccttgagcaaaaagccttcgtgtctgtaagtgcccgaggctcaggagagctggggctcccactcgcggcagacaggcccgcgtccaccctgcgtccaccccggcccggcggcagcacggtgccagtcatctgcatgtgctcctgcggcgtgggggtttgtagacttggaaaaccctgttggcagaaagttaagagctcccaggcctgaaggcaggacacagtgccagcagggggccgtttgcccccatgtgtaagggaggcagggcccagctctcccacggcgaggtga

SEQ ID NO: 41 - RNA, 22 nt, Homo sapiens
caguuaucacagugcugaugcu

SEQ ID NO: 42 - RNA, 75 nt, Homo sapiens
ugcccuggcucaguuaucacagugcugaugcugucuauucuaaagguacaguacugugauaacugaaggauggca

SEQ ID NO: 43 - DNA, 669 bp, Homo sapiens
tgctggaagcttactgcatatttgatgtattagagtgaaaacctaatcatgcagttgttcatcctcattaatatggataagtcatgtgttcatctttcattctaatttaattcaactgggccttttaatatttcagcctcaccacttgctgggctctgatccttctttttcttctgcctcctcacgtctccaaccagaaggtgatcttttagtccttcacttcatggggagccttcagagagagtaatgcagccaccagaaaggatgccgttgaccgacacagtgactgacaggctgccctggctcagttatcacagtgctgatgctgtctattctaaaggtacagtactgtgataactgaaggatggcagccatcttaccttccatcagaggagcctcaccgtacccaggaagaaagaaggtgaaagaggaatgtgaaacaggtggctgggacccagaaaccctcttaccctgcacctctgtcatacttctcccggggcatagggagagttattctgcttctctttgccttgttttgtaacatggggtagttgttggtgcagccatgttgtgctgagtgaacatatattaagatctttggaacctttaggagactgaaaataggtaagtatgaattagtatttctggaatggtattcagagaacttcg

SEQ ID NO: 44 - RNA, 22 nt, Homo sapiens
uagcagcacguaaauauuggcg

SEQ ID NO: 45 - RNA, 89 nt, Homo sapiens
gucagcagugccuuagcagcacguaaauauuggcguuaagauucuaaaauuaucuccaguauuaacugugcugcugaaguaagguugac

SEQ ID NO: 46 - DNA, 659 bp, Homo sapiens
atctccttaaaaataattatgcatattacatcaatgttataatgtttaaacatagatttttttacatgcattctttttttcctgaaagaaaatattttttatattctttaggcgcgaatgtgtgtttaaaaaaaataaaaccttggagtaaagtagcagcacataatggtttgtggattttgaaaaggtgcaggccatattgtgctgcctcaaaaatacaaggatctgatcttctgaagaaaatatatttctttttattcatagctcttatgatagcaatgtcagcagtgccttagcagcacgtaaatattggcgttaagattctaaaattatctccagtattaactgtgctgctgaagtaaggttgaccatactctacagttgtgttttaatgtatattaatgttactaatgtgttttcagttttattgatagtcttttcagtattattgataatcttgttatttttagtatgattctgtaaaaatgaattaatactaatttttcagatgtatcatctcttaaaatactgtaattgcaatttaataattgtattgaatgccatcaagtttttttaaaaagcttatgcagcattagaggaatttattttaatgcacatttatattcaacatagacattaattcagatttttacttggga

SEQ ID NO: 47 - RNA, 22 nt, Homo sapiens
aacauucaaccugucggugagu

SEQ ID NO: 48 - RNA, 110 nt, Homo sapiens
cggaaaauuugccaaggguuugggggaacauucaaccugucggugaguuugggcagcucaggcaaaccaucgaccguugaguggacccugaggccuggaauugccauccu

SEQ ID NO: 49 - DNA, 688 bp, Homo sapiens
ggtaatctgcacctccaggatcactttgtgaattacttaaggagcgggcttgaggccagcactcccctgcactgctacatctccatccccatagcaaaggctacattttatttactagcatttatatttgccttgtgtttttcccagatctaggtgcaaacagcacctgaaaagcgtttgtttatttcatgagagaaaaggggttccttactctctcctcctctccctcttcatgctctctggttccctgccacctaccccacccccgactccaggtcccggaaaatttgccaagggtttgggggaacattcaacctgtcggtgagtttgggcagctcaggcaaaccatcgaccgttgagtggaccctgaggcctggaattgccatcctcctgccggtgactctgaccttccagatctaggggggcctggggagcccccaatccagcctgggcacgtcccctcccctaggccacagccgaggtcacaatcaacattcattgttgtcggtgggttgtgaggactgaggccagacccaccgggggatgaatgtcactgtggctgggccagacacggcttaaggggaatggggactggggacaggaccccccaccgccacagtcactcagcctgttttttgccctgaccccaaccactcctctttggagaggagagctggtgtctggaatc

SEQ ID NO: 50 - RNA, 22 nt, Homo sapiens
aaaguucugagacacuccgacu

SEQ ID NO: 51 - RNA, 68 nt, Homo sapiens
gaggcaaaguucugagacacuccgacucugaguaugauagaagucagugcacuacagaacuuugucuc

SEQ ID NO: 52 - RNA, 23 nt, Homo sapiens
agcagcauuguacagggcuauca

SEQ ID NO: 53 - RNA, 81 nt, Homo sapiens
cucucugcuuucagcuucuuuacaguguugccuuguggcauggaguucaagcagcauuguacagggcuaucaaagcacaga